(19) United States
(12) Patent Application Publication
(10) Pub. No.: US 2011/0257517 A1
(43) Pub. Date: Oct. 20, 2011

(54) Patient-Specific Seizure Onset Detection System

(76) Inventors: John V. Guttag, Lexington, MA (US); Ali Hossam Shoeb, Winchester, MA (US); Blaise Bourgeois, Newton, MA (US); S. Ted Treves, Wellesley, MA (US); Steven C. Schachter, Sharon, MA (US); Herman A. Edwards, Watertown, MA (US); John Connolly, Norton, MA (US)

(21) Appl. No.: 13/153,819

(22) Filed: Jun. 6, 2011

Related U.S. Application Data
(63) Continuation of application No. 11/140,551, filed on May 27, 2005, now abandoned.
(60) Provisional application No. 60/575,280, filed on May 27, 2004.
(60) Provisional application No. 60/575,125, filed on May 27, 2004.

(51) Int. Cl. A61B 5/0476; A61B 6/03; A61B 6/00

(52) U.S. Cl. 600/425; 600/544; 600/431

(57) ABSTRACT

The present invention provides methods and systems for patient-specific seizure onset detection. In one embodiment, at least one EEG waveform of the patient is recorded, and at least one epoch (sample) of the waveform is extracted. The waveform sample is decomposed into one or more subband signals via a wavelet decomposition of the waveform sample, and one or more feature vectors are computed based on the subband signals. A seizure onset can then be identified based on classification of the feature vectors to a seizure or a non-seizure class by comparing the feature vectors with a decision measure previously computed for that patient. The decision measure can be derived based on reference seizure and non-seizure EEG waveforms of the patient. In another aspect, similar methodology is employed for automatic detection of alpha waves. In other aspects, the invention provides diagnostic and imaging systems that incorporate the above seizure-onset and alpha-wave detection methodology.

### RELATED APPLICATIONS

The present application claims priority to a provisional application entitled “Patient-Specific Seizure Onset Detection,” filed on May 27, 2004 and having a Ser. No. 60/575,280. The present application also claims priority to a provisional application entitled “Use of Seizure Detector To Activate A Vagus Nerve Stimulator,” filed on May 27, 2004 and having a Ser. No. 60/575,125.

### BACKGROUND OF THE INVENTION

The present invention relates generally to methods and systems for automatic detection of selected changes in a patient's EEG waveforms, and by way of non-limiting applications to seizure detection as well as various diagnostic and therapeutic applications that employ these methods and systems.

Approximately one percent of the world's population exhibits symptoms of epilepsy, a serious disorder of the central nervous system that predisposes those affected to recurrent seizures. A seizure is a sudden breakdown of the neuronal activity of the brain that precipitates an involuntary alteration in behavior, movement, sensation, or consciousness. The confusion, loss of consciousness, or lack of muscle control that can accompany certain seizure types can lead to serious injuries, such as broken bones, head injuries, burns and even deaths.

A number of imaging and diagnostic systems for localizing the focus of a seizure and ameliorating the symptoms of a seizure are known. The optimal functioning of many such systems, however, requires accurate and timely detection of a seizure. Conventional seizure detection methods and devices, however, suffer from a number of shortcomings in this regard. For example, such devices can exhibit high false-positive rates, a high rate of missed seizures, significant delays between electrographic onset of a seizure and its detection, or highly intensive computations that can limit real-time processing of EEG data.

Accordingly, there is a need for enhanced methods and systems for detecting seizures and for enhanced diagnostic and therapeutic applications related to epilepsy. There is also a need for enhanced diagnostic and imaging methods for use in epileptic patients.

### SUMMARY OF THE INVENTION

The present invention generally provides automated, patient-specific methods and systems for the detection of epileptic seizure onset from electroencephalographic (EEG) brain waveforms. The seizure detection methods and systems of the invention utilize the consistency of an individual patient's seizure and non-seizure EEG waveforms to identify a seizure onset in that patient. As discussed in more detail below, in some embodiments, a feature vector that captures the morphology and spatial distribution of at least one EEG epoch of a patient is constructed. The feature vector can then be classified using a previously-obtained measure. For example, the Support Vector Machine classification algorithm can be employed. Alternatively, a statistical approach can be adopted to classify the feature vector. In other embodiments, a plurality of feature vectors are generated and their spatial inter-relationships are examined after their assignments to a seizure or a non-seizure class. A seizure onset can then be identified based on these classifications and selected temporal and spatial constraints.

In other aspects, a variety of diagnostic and therapeutic systems are disclosed that incorporate the seizure detection methods and systems according to the teachings of the invention. Some examples of such systems include, without limitation, systems for performing ictal SPECT imaging and stimulating the vagus nerve.

In one aspect of the invention, methods of detecting an onset of an epileptic seizure in a patient are disclosed, which can comprise the steps (not necessarily sequentially) of recording at least one waveform indicative of a patient's brain activity, extracting at least one sample of the waveform, applying a selected transformation to the sample so as to derive at least one feature vector, and classifying the feature vector as belonging to a non-seizure class or a seizure class based on comparison with at least one reference value previously identified for the patient. The waveform can correspond to an invasive or non-invasive EEG waveform channel of the patient. A sample of the waveform (or a sampled waveform) refers to a temporal portion (an epoch) of the waveform—a segment of the waveform observed within a time period. The feature vector can include one or more values indicative of the morphology of the waveform sample.

The method can further include the step of identifying an onset of a seizure if the feature vector is classified as belonging to a seizure class, or by identifying an onset of a seizure if feature vectors corresponding to at least two consecutive waveform samples are classified as belonging to the seizure class. The seizure class can represent EEG activity observed in the patient during onset of a seizure and the non-seizure class can represent EEG activity observed during a period other than a seizure onset period, e.g., normal EEG waveforms observed in the patient in different states of consciousness or artifact-contaminated EEG waveforms observed in the patient in different states of consciousness.

The reference value used during the classifying step can be derived based on a condition associated with the seizure class and a condition associated with the non-seizure class. The classifying step can further comprise assigning the feature vector to a non-seizure class or a sub-class of the seizure class.

In one embodiment, the feature vector is indicative of energy contained within at least two subband signals (herein also referred to as subbands) having frequency content lying in two noncongruent bands and derived from the waveform sample and the step of applying a transformation to the waveform sample can further entail time-frequency decomposition (e.g., a wavelet decomposition) of the waveform to generate a plurality of subband signals. By way of example, the subband signals can be derived from analysis of the waveform at a plurality of time-frequency scales defined by the contraction or dilation of a selected wavelet. Two noncongruent frequency bands can be two bands whose centers (center frequencies) are offset relative to one another. Such noncongruent frequency bands can be disjoint or partially overlapping. In one approach, the waveform sample can be decomposed into the subband signals to generate a feature vector. For example, the feature vector can be formed based on energy contained in one or more of the subband signals. More preferably, the method can further include computing a function of energy contained within each of the subband signals for generating the feature vector. In some applications, it may be preferable to compute the energy of each subband signal as a logarithmic function. In many embodiments, the subband signals can encompass components of the waveform at frequencies in a range of about 0.5 to about 25 Hz.

In another aspect of the invention, the classifying step can further comprise computing a probability that a derived feature vector belongs to a seizure class. Alternatively, the classifying step can further comprise identifying one or more support vectors and computing their associated classification parameters based on previously derived reference feature vectors. The reference feature vectors can be generated from previously-obtained brain waveforms of the patient associated with seizure and non-seizure classes. Classification can also include computing a decision hyperplane based on the support vectors and assigning the feature vector to a seizure or non-seizure class based on location of the feature vector relative to the hyperplane. In many embodiments, the hyperplane is defined in a higher dimensional space than that of the feature vectors.

In one embodiment, the wavelet decomposition of the waveform sample can comprise passing each sampled waveform through a bank of filters, such as an iterated or a polyphase wavelet filter bank.

In another aspect, the present invention encompasses a method of detecting an onset of an epileptic seizure in a patient, comprising the steps (not necessarily sequentially) of generating at least one reference feature vector based on one or more sample brain waveforms of the patient, with at least one prior waveform being designated as belonging to a seizure class and at least one prior waveform designated as belonging to a non-seizure class, monitoring at least one EEG waveform channel of the patient, deriving at least one feature vector based on at least one sample of the monitored EEG waveform, and classifying the derived feature vector as belonging to the seizure class or the non-seizure class based on comparison with the reference seizure onset and non-seizure feature vectors. The classifying step can further include comparing the derived feature vector with a decision measure obtained from the reference feature vectors. The method can further entail identifying onset of seizure in the patient based on the classification of the feature vector.

In one embodiment, the seizure class comprises EEG waveforms of the patient observed during onset of a seizure and the non-seizure class comprises EEG waveforms of the patient observed during a period other than a seizure onset period. For example, the non-seizure class can comprise normal EEG waveforms observed in the patient in different states of consciousness.

In this approach, support vectors can be identified based on the reference feature vectors and the method can further comprise computing a decision hyperplane based on the support vectors and assigning feature vectors to the seizure or the non-seizure class based on location of the feature vector relative to the hyperplane.

In another aspect of the invention, methods are disclosed for detecting an onset of an epileptic seizure in a patient, comprising the steps (not necessarily sequentially) of monitoring concurrently a plurality of EEG waveform channels of the patient, extracting a sample of each of the waveforms during a common time period, applying a selected transformation to each sample waveform so as to derive a feature vector corresponding to that sample, and classifying each of the feature vectors as belonging to a seizure class or a non-seizure class based on comparison of the feature vectors with reference feature vectors previously obtained from reference EEG waveforms of the patient, at least one of the reference waveforms belonging to the seizure class and at least one of the reference waveforms belonging to the non-seizure class. This method can further comprise identifying a seizure onset based on a subset of the feature vectors being classified as belonging to the seizure class, e.g., based on spatial constraints derived for the patient. Again, the method can further comprise selecting the transformation to be a wavelet decomposition.

In a further aspect of the invention, methods are disclosed for detecting an onset of an seizure in a patient, comprising the steps (not necessarily sequentially) of monitoring a plurality of waveform channels corresponding to brain activity of the patient, extracting samples of the channel waveforms, and, for each channel, generating a feature vector by applying a selected transformation to the channel, grouping the feature vectors into a composite feature vector, and then classifying the composite feature vector as belonging to a seizure class or a non-seizure class based on comparison with a reference value previously identified for the patient. The reference feature vectors can be generated by applying a transformation to the reference EEG waveform samples from the channels, the reference samples including at least one waveform belonging to the seizure class and at least one waveform belonging to the non-seizure class. In one embodiment, support vectors can be identified based on the reference feature vectors.

The method can further comprise computing decision boundaries for use in the classifying step based on the support vectors, wherein the classifying step comprises comparing the composite feature vector with the decision boundaries and the transformation comprises a time-frequency transformation. This method can further comprise the step of generating the feature vector of a channel waveform by wavelet decomposition of the sampled waveform into a plurality of subband signals, and computing energy contained in each subband signal. In this approach, the feature vector can be generated by calculating a function of the computed energy.

In general, a variety of analytical methods can be employed to derive the feature vector. Some examples of such methods include, without limitation, the Matching Pursuit Algorithm with a dictionary of basis functions such as Gabor Atoms, Wavelet Packets, Continuous Wavelet Transform and Discrete Wavelet Transform (which is employed in exemplary embodiments discussed below).

In a further aspect of the invention, methods are disclosed for detecting an onset of a seizure in a patient, comprising the steps (not necessarily sequentially) of detecting an onset of an epileptic seizure in a patient by obtaining samples of a plurality of EEG channel waveforms of the patient, decomposing each sampled waveform (e.g., via a wavelet decomposition) into a plurality of subband signals, computing a plurality of feature vectors, each feature vector corresponding to one of the sampled waveforms and being computed based on the subband signals associated with that waveform, and classifying each feature vector as belonging to a seizure class or a non-seizure class based on comparison with a measure derived from at least one reference value previously identified for the patient. Accordingly, seizure onset can be identified based on a subset of the feature vectors being classified as belonging to the seizure class. In one embodiment, the classifying step can employ a maximum likelihood classifier having kernel functions based on the reference feature vectors.

In another aspect, the invention encompasses a method of detecting onset of seizure in a patient, comprising the steps (not necessarily sequentially) of providing a classifier with reference EEG waveforms of the patient, wherein at least one of the reference waveforms is designated as belonging to a seizure class and at least one of which is designated as belonging to a non-seizure class, utilizing the classifier to generate a decision measure for that patient based on the reference EEG waveforms, thereby training the classifier. The method further comprises recording at least one EEG waveform channel of the patient, deriving at least a feature vector based on at least a sample of the observed EEG waveform, and utilizing the trained classifier to assign the feature vector to the seizure class or the non-seizure class, and identifying onset of a seizure based on the classification of the feature vector.

In this method the seizure class can comprise EEG waveforms of the patient observed during onset of a seizure and the non-seizure class can comprise EEG waveforms of the patient observed during a period other than a seizure onset period. The step of utilizing the classifier to generate a decision measure can further comprise utilizing the classifier to decompose (e.g., via wavelet decomposition) each reference EEG waveform into at least one subband signal, and utilizing the subband signal to generate at least one reference feature vector, deriving support vectors based on the reference feature vector, and computing the decision measure based on the support vectors.

In another aspect, the invention provides a method of processing an EEG waveform of a subject that comprises: recording at least one EEG channel waveform of the subject, extracting at least one sample (epoch) of the waveform, generating at least one feature vector based on the sample, and classifying the feature vector as belonging to a first EEG class or a second EEG class. The method can further comprise identifying a change in the EEG waveform based on the above classification of at least two consecutive samples of the waveform.

In a further aspect of the invention, methods are disclosed for detecting onset of a seizure in a patient, comprising the steps (not necessarily sequentially) of recording at least one waveform indicative of brain activity of the patient, applying a transformation to at least a sample of the waveform so as to generate at least one feature vector, classifying the feature vector as belonging to one of (i) a seizure class of a first type, (ii) a seizure class of a second type, or (iii) a non-seizure class, and identifying onset of a seizure of the first type or the second type based on the classification of the feature vector.

In this method, the classifying step can further comprise applying the feature vector to a first classifier trained on reference brain waveforms of the patient, at least one of which belongs to the seizure class of the first type and at least one of which belongs to the non-seizure class, to classify the feature vector as belonging to the seizure class of the first type or to the non-seizure class, and applying the feature vector to a second classifier trained on reference brain waveforms of the patient, at least one of which belongs to the seizure class of the second type and at least one of which belongs to the non-seizure class, to classify the feature vector as belonging to the seizure class of the second type or to the non-seizure class.

The seizure class of the first type can comprise a patient's brain waveform corresponding to onset of a seizure of the first type while the seizure class of the second type can comprise a patient's brain waveform corresponding to onset of a seizure of the second type. It should be understood that the methods can be similarly applied to identify seizure onsets corresponding to more than two different types of seizure. In fact, classifiers trained on any desired number of seizure types can be employed.

In yet another aspect of the invention, systems are disclosed for detecting onset of an epileptic seizure in a patient, comprising: a feature extractor operating on at least one sampled EEG waveform recording patient neuroactivity to compute at least a feature vector corresponding to the sampled waveform, and a classifier capable of being trained on reference EEG waveforms of the patient so as to identify onset of a seizure based on assigning the feature vector to a seizure or a non-seizure class, wherein at least one of the reference EEG waveforms is associated with a seizure class and at least one of the reference EEG waveforms is associated with a non-seizure class.

In such systems the classifier can be adapted to receive the reference feature vectors and to generate a decision measure based on the reference feature vectors for that patient, whereby the classifier can employ the decision measure to assign the sample waveform to a seizure or a non-seizure class.

The feature extractor decomposes the sampled waveform into a plurality of subband signals for computing the feature vector corresponding to that waveform and, optionally, the feature extractor can compute an energy contained within each of the plurality of the subband signals for computing the feature vector associated with that waveform.

In another aspect of the invention, systems are disclosed for detecting onset of an epileptic seizure in a patient, comprising: a feature extractor operating on sampled EEG waveforms of the patient from a plurality of channels to compute, for each channel, a feature vector, the extractor grouping the feature vectors into a composite feature vector, and a classifier trained on reference EEG waveforms of the patient, at least one the reference EEG waveforms belonging to a seizure class and at least one of the reference EEG waveforms belonging to a non-seizure class, wherein the classifier identifies onset of a seizure based on classification of the feature vector as belonging to a seizure or a non-seizure class.

In one embodiment, a system according to the invention for detecting an onset of a seizure in a patient, can comprise: a computing device and at least one decision reference parameter stored in the computing device derived from reference brain waveforms of the patient, at least one of the reference waveforms belonging to a seizure class and at least one of the reference waveforms belonging to a non-seizure class. The computing device can have at least one input port capable of receiving waveform data corresponding to brain activity of the patient, whereby the computing device can apply a selected transformation to the input channel data to generate at least a feature vector and classifies the feature vector as belonging to the seizure class or the non-seizure class by comparison with the decision parameter. The system can further include instructions stored in the computing device for executing the selected transformation and instructions for determining an onset of a seizure based on the classification of the feature vector.

The computing device can indicate onset of a seizure when feature vectors corresponding to at least two successive samples of the waveform data are classified as belonging to the seizure class. The decision parameter can comprise a hyperplane constructed based on one or more support vectors derived from reference feature vectors generated based on the reference brain waveforms.

The transformation carried out by the system can comprise a wavelet decomposition of the waveform channel data into a plurality of subband signals and, optionally further comprise computing energy contained within the subband signals.

In another aspect, the invention provides a system for detecting onset of an epileptic seizure in a patient that comprises a feature extractor operating on at least one sampled EEG waveform indicative of brain activity of the patient to compute a feature vector, and two or more classifiers in communication with the feature extractor and each trained on previously-obtained reference brain waveforms of that patient to classify the feature vector as belonging to a seizure class of a given type or a non-seizure class. For example, the system can include a first classifier trained to classify the feature vector as belonging to a non-seizure class or a seizure class of a first type and a second classifier trained to classify the feature vector as belonging to a non-seizure class or a seizure class of a second type.

In some embodiments, one or more classifiers can detect seizure onsets (without necessarily determining the types of the seizures) and other classifiers (one or more) coupled to the first set can determine the type(s) of the detected seizures.

In a related aspect, each classifier can indicate onset of a seizure of the type associated therewith based on its classification of the feature vector.

In another aspect, the invention provides a method for detecting onset of a subject's brain alpha waves, comprising: monitoring a waveform from at least one channel of an EEG measurement of the patient's brain activity, extracting at least one sample of the waveform, generating at least one feature vector based on a transformation of the sampled waveform, and classifying the feature vector as belonging to a non-alpha wave class or an alpha-wave class based on comparison of the feature vector with a reference value previously obtained for the subject.

In a related aspect, the above method of detecting a subject's brain alpha wave can further comprise identifying onset of an alpha wave if the feature vector is classified as belonging to the alpha wave class. In some embodiments, an onset of an alpha wave is identified (declared) if a selected number of feature vectors corresponding to consecutive waveform samples are classified as belonging to the alpha wave class.

In the above method, the alpha wave class can comprise the patient's brain waveforms during onset of an alpha wave and the non-alpha wave class can comprise the patient's brain waveforms during periods other than onset of an alpha wave. For example, the non-alpha wave class can include waveforms that do not exhibit alpha wave characteristics

In another aspect, the method comprises issuing a notification (e.g., an alarm) upon detection of the onset of an alpha wave.

In yet another aspect, the transformation applied to the sampled waveform in the above method of detecting an alpha wave onset can comprise wavelet decomposition of the sampled waveform into a plurality of subband signals and computing energy contained in each subband signal.

The reference value utilized in above method of detecting alpha wave onset can correspond to at least one decision boundary. In some embodiments, the decision boundary can be computed by utilizing a plurality of support vectors, the support vectors being identified based on one or more reference feature vectors computed from alpha wave and non-alpha wave sampled waveforms of the subject.

Further, in the above method of detecting onset of alpha waves, the EEG measurements can be selected to be non-invasive or invasive measurements.

In other aspects, systems for detecting an onset of alpha waves are disclosed. Such a system can comprise: a computing device, and at least one decision reference parameter stored in the computing device derived from reference brain waves of the subject belonging to an alpha wave class and a non-alpha wave class. The computing device can have at least one input port for receiving input waveform data corresponding to brain activity of the patient, and can apply a selected transformation to the input waveform data to generate at least a feature vector. Further, the computing device can classify the feature vector as belonging to the alpha wave class or the non-alpha wave class by comparison with the decision parameter.

In another aspect, changes in one or more EEG channel waveforms of a patient can be automatically detected during a time period so as to identify a sequence of events during that period. For example, the sequence of events can include a seizure onset followed by the remainder of the seizure and subsequent cessation of the seizure. Alternatively, the sequence of events can correspond to temporal EEG changes related to emergence of alpha waves and their subsequent cessation. In some embodiments, such automatic detection of events can be accomplished by monitoring the energy contained in one or more subband signals derived by a time-frequency decomposition of EEG waveform samples. Such automatic identification of a sequence of events can be useful, for example, in determining the status of a patient during different epochs of a given time period.

In other aspects, methods and systems for applying stimuli to a patient in response to detection of a seizure onset are disclosed. One such method for applying a stimulus to a patient comprises: monitoring at least one waveform channel indicative of a patient's brain activity, generating at least one feature vector based on at least a sample of the waveform, detecting onset of a seizure based on classifying the feature vector as belonging to a seizure class or a non-seizure class by comparison with a measure derived from previously-observed reference brain waveforms of that patient, and applying a stimulus to the patient in response to a detected seizure onset.

At least one of the reference waveforms can belong to a seizure class and at least one of the reference waveforms can belong to a non-seizure class.

In some embodiments of the above method of applying a stimulus to a subject in response to a detection of a seizure onset, the sample of the waveform is decomposed (e.g., via wavelet decomposition) into at least one subband signal and the feature vector is computed as a function of energy contained within that subband.

In a related aspect, in the above method of applying a stimulus to a subject, a seizure onset is identified upon classifying feature vectors corresponding to at least two consecutive samples of the waveform to the seizure class.

In some embodiments, the method of applying a stimulus further comprises generating reference feature vectors based on reference seizure and non-seizure waveforms of the subject, and identifying a plurality of support vectors and their associated classification parameters based on the reference feature vectors. A decision hyperplane can then be computed based on the support vectors and the feature vector can be assigned to a seizure or a non-seizure class based on location of the feature vector relative to the hyperplane.

In a related aspect, the step of applying a stimulus comprises stimulating the patient's vagus nerve, e.g., by utilizing a vagus nerve stimulator, so as to prevent or lessen the occurrence of symptoms and/or signs of the seizure, and/or ameliorate the severity of the seizure or the post-ictal symptoms and/or signs. The stimulation of the vagus nerve can also result in shortening the duration of the seizure and/or the post-ictal symptoms. More generally, the step of applying a stimulus can comprise stimulating one or more cranial nerves of the patient so as to prevent or lessen the duration and severity of the occurrence of symptoms and/or signs of the seizure. For example, the stimulus can be applied to the subject's glossopharyngeal nerve. In other embodiments, a stimulus can be applied to selected areas of the subject's skin so as to prevent or lessen the occurrence of symptoms and/or signs of seizure. Other types of stimulation suitable in the practice of the invention are discussed below.

In the above method of applying a stimulus to a subject in response to detection of a seizure onset, the subject's brain waveform can be a non-invasive or an invasive EEG waveform.

In another aspect, the invention discloses a system for applying a stimulus to a patient, comprising: a device for monitoring at least one EEG waveform of the patient, a seizure detector receiving the monitored EEG waveform and detecting an onset of a seizure based on classifying a feature vector derived from a sample of the waveform as belonging to a seizure class or a non-seizure class, the detector performing the classification based on comparison of the feature vector with a decision measure derived from previously-obtained reference EEG waveforms of the patient. The system further comprises a stimulator for applying a stimulus to the patient in response to identification of the seizure onset.

In some embodiments, the seizure detector of the above system for applying a stimulus to a patient comprises: a feature extractor operating on the sampled EEG waveform to compute a feature vector, and a classifier trained on reference EEG waveforms of the patient, the classifier assigning the feature vector to a seizure or a non-seizure class. The seizure class can comprise the patient's onset EEG waveforms, and the non-seizure class can comprise the patient's brain waveforms during periods other than seizure onset periods, e.g., normal EEG waveforms.

In some embodiments, the stimulator comprises a vagus nerve stimulator (VNS). The VNS can be optionally in communication with the detector, wherein the detector can cause activation of the stimulator to apply a selected stimulus to the patient's vagus nerve upon detection of a seizure onset. The stimulus can be, for example, an electrical excitation applied to the subject's vagus nerve. More generally, the stimulator can apply an excitation to one or more cranial nerves of the patient, such as the glossopharyngeal nerve. Alternatively, the stimulator can apply a selected excitation to the patient's brain tissue, or selected areas of the patient's skin, in response to detection of a seizure onset.

In other aspects, portable devices for applying a stimulus to a subject's vagus nerve are disclosed. Such a portable device can comprise: a seizure detector having at least one port for receiving at least one EEG waveform of the patient, the detector generating a feature vector based on at least a sample of the waveform and identifying an onset of a seizure based on classification of the feature vector as belonging to a seizure class or a non-seizure class. The portable device can further comprise a stimulator device in communication with the detector and adapted to apply a selected stimulus to the patient's vagus nerve. The detector can trigger the stimulator device in response to detection of a seizure onset to apply a stimulus to the patient's vagus nerve.

In a related aspect, in the above portable device, the detector performs the classification by comparison of the feature vector with one decision parameter derived from previously obtained reference EEG waveforms of the patient. At least one of the reference EEG waveforms can belong to a seizure class and at least one of the reference EEG waveforms belongs to a non-seizure class.

In some embodiments, the portable device can further comprise a switch coupled to the detector and the stimulator. The detector can trigger the switch so as to activate the vagus nerve stimulator. The switch can comprise, for example, an electromagnet generating a sufficiently strong magnetic field upon being triggered by the detector so as to activate the vagus nerve stimulator. Further, the detector can cause the de-activation of the vagus nerve stimulator (e.g., by turning off the switch), for example, in response to detection of the cessation of a seizure event.

In some embodiments, the seizure detector of the above portable device can comprise: a computing device, and at least one decision reference parameter stored in the computing device derived from reference brain waveforms of the patient, wherein at least one of the reference waveforms belongs to a seizure class and at least one of the reference waveforms belongs to a non-seizure class. The computing device can further comprise at least one input port capable of receiving waveform data corresponding to brain activity of the patient. The computing device can apply a selected transformation to the input waveform data to generate the feature vector, and can classify the feature vector as belonging to the seizure class or the non-seizure class by comparison of the feature vector with the decision parameter. The computing device can identify a seizure onset based on the classification.

In another aspect, a system for delivering a therapeutic agent to a patient is disclosed that comprises: a seizure detector adapted to receive at least one EEG waveform channel of the patient to generate at least a feature vector characterizing the waveform, the detector detecting an onset of a seizure by classifying the feature vector as belonging to a seizure class or a non-seizure class. The delivery system further comprises a device for delivering a therapeutic agent to the patient in response to detection of the seizure onset by the detector.

In some embodiments, the seizure detector of the delivery system performs the classification based on comparison of the feature vector with a decision measure derived from previously-obtained reference waveforms of the patient, wherein at least one of the reference waveforms belongs to a seizure class and at least one of the reference waveforms belongs to a non-seizure class. The seizure class comprises reference waveforms corresponding to onset of a seizure and the non-seizure class comprises reference waveforms corresponding to periods other than seizure onset periods.

In related aspects, the delivery device can deliver the therapeutic agent to the patient at a selected time after detection of the seizure onset, and the delivery system can further comprise a device for acquiring the EEG waveform channel.

In other aspects, methods and systems for acquiring diagnostic data are disclosed. In one such method of acquiring diagnostic data from a patient comprises: monitoring at least one waveform indicative of brain activity of the patient, detecting an onset of an epileptic seizure by classifying at least one feature vector corresponding to a sample of the waveform as belonging to a seizure class or a non-seizure class, the classification being based on comparison of the feature vector with a measure derived from previously-observed reference waveforms of that patient, and acquiring diagnostic data in response to detection of a seizure onset. At least one of the reference waveforms can be a member of the seizure class and at least one of the reference waveforms can be a member of the non-seizure class.

In the above diagnostic data acquisition method, the brain waveform can be a non-invasive, or an invasive EEG channel waveform. In some embodiments, a seizure onset can be identified (declared) when feature vectors corresponding to at least two consecutive samples of the waveform are classified as belonging to the seizure class.

In some embodiments of the above method of acquiring diagnostic data, the waveform sample is decomposed into a plurality of subband signals, and the feature vector corresponding to a sampled waveform is computed based on energy contained in each of the subbands. The subband signals can encompass components of the waveform in a frequency range of about 0.5 to about 25 Hz.

In another aspect, the above method of acquiring diagnostic data can further comprise deriving reference feature vectors based on the previously-observed reference waveforms of the patient. The reference feature vectors can be utilized to identify support vectors, which can be employed to construct one or more decision boundaries corresponding to the measure with which the observed feature vector is compared.

In some embodiments, the measure can comprise a statistical measure obtained by applying a maximum likelihood classifier to the reference feature vectors derived from one or more reference EEGwaveforms, wherein the classifier has kernel functions based on the reference feature vectors.

In related aspects, the data acquisition step can comprise obtaining an image or a sample related to a metabolic or hormonal or other physiological activity in a selected anatomical portion of the patient and/or acquiring a neurological image. In some embodiments, the data acquisition step can comprise obtaining an image related to neural activity in at least a portion of the patient's brain. The acquired diagnostic data can be utilized, for example, to determine the location of the site of a seizure onset. In some embodiments, the data acquisition step can comprise obtaining a single-photon-emission computed tomography (SPECT) image of the patient's brain. The SPECT image can be employed, for example, to localize the focus of the onset of a seizure. Alternatively, the data acquisition step can comprise obtaining a functional magnetic resonance image (fMRT), or a near infrared spectral image of the patient's brain. Moreover, in some embodiments, the data acquisition step can comprise obtaining a positron emission tomography (PET) image of the patient's brain. In some other embodiments, the data acquisition step can include utilizing magnetoencephalography, a non-invasive diagnostic modality for functional brain mapping.

As discussed in more detail below, in some embodiments, upon detection of a seizure onset in a subject, one or more waveforms from one or more channels identified as exhibiting seizure activity as well as one or more reference EEG waveforms of that subject are presented to a medical professional (e.g., via a display device coupled to the detector), to facilitate identification of false-positive detections. For example, the reference EEG waveforms can correspond to previously-observed seizure events of that subject. Alternatively or in addition, the reference EEG waveforms can correspond to inter-ictal discharges previously observed in that subject to permit the medical professional to determine whether the detected seizure corresponds to such an inter-ictal discharge (and hence a false-positive). It should, however, be understood that in some applications, detection of such inter-ictal discharges may be desired (e.g., in some cases, a stimulation can be applied to the subject in response to such inter-ictal discharge detections).

In a related aspect, the above method of acquiring diagnostic data can further comprise delivering a diagnostic agent, e.g., a radiotracer or any other suitable agent, to the patient upon detection of onset of a seizure so as to facilitate the diagnostic data acquisition. In some embodiments, the requisite dose of the diagnostic agent is automatically computed upon detection of a seizure onset, and is communicated to a device that delivers the agent.

In another aspect, the invention discloses a method of correlating seizure events of a patient with one or more images of the patient that comprises: monitoring at least one EEG waveform of the patient during a selected time period, obtaining at least one image of the patient during at least a portion of the time period, and detecting seizure events, if any, of the patient during the time period by classifying at least one feature vector, obtained based on at least a sample of the monitored waveform, as belonging to a seizure class or a non-seizure class based on comparison with a measure derived from previously-obtained reference waveforms of the patient. The method further comprises temporally correlating at least a portion of a detected seizure event with at least one time segment of the image.

The EEG waveform channel can be any of a non-invasive channel or an invasive channel. Further, at least one of the reference waveforms can belong to the seizure class and at least one of the reference waveforms can belong to the non-seizure class.

In some embodiments of the above method of correlating seizure events of a patient with one or more images of the patient, the feature vector is generated by wavelet decomposition of the waveform sample into a plurality of subband signals and computing a function of energy contained within each subband signal.

In a related aspect, the image that is correlated with the patient's seizure events can include a video image, a SPECT image, and fMRI image or any other suitable image of the patient. Further, a seizure event can correspond to a seizure onset, or any portion or the entire duration of a seizure.

In another aspect, a method of determining the focus of a patient's epileptic seizure is disclosed that comprises: extracting at least a sample of at least one waveform monitoring neural activity in a selected brain portion of the patient, identifying an onset of an epileptic seizure of the patient by classifying at least one feature vector derived based on wavelet decomposition of the waveform sample as belonging to a seizure or a non-seizure class, the classification being based on comparison of the feature vector with a measure derived from previously-obtained reference brain waveforms of the patient, and delivering a diagnostic agent to the patient upon detection of the onset of a seizure.

In a related aspect, an image of the diagnostic agent can be generated, and the image can be employed to determine the focus of site of the seizure onset. The diagnostic agent can be, without limitation, a radiotracer or a dye. Further, the image can be selected to be a SPECT image of the patient's brain.

In another aspect, a system for determining a focus of seizure onset of an epileptic seizure of a patient is disclosed that comprises: a device for monitoring at least one EEG waveform channel of the patient, and a patient-specific seizure detector for detecting an onset of a seizure by classifying at least a feature vector derived from at least a sample of the waveform as belonging to a seizure or a non-seizure class, wherein the detector performs the classification by comparing the feature vector with a measure computed based on one or more reference feature vectors previously derived for that patient. The system can further include a pump for delivering a radiotracer to the patient in response to detection of a seizure onset by the detector. In some embodiments, the system can further include a device for ensuring, before activation of the pump, that an intravenous (IV) line coupled to the patient for injecting the radiotracer is functioning properly.

In some embodiments, the detector comprises a feature extractor for wavelet decomposition of the waveform sample into at least one subband signal and computing the feature vector as a function of energy contained within the subband signal, and a classifier trained on reference EEG waveforms of the patient to assign the feature vector to a seizure or a non-seizure class.

In some embodiments, the detector can compute the feature vector as a composite of a plurality of feature vectors, each corresponding to a sample of one of a plurality of EEG waveforms of the patient.

In a related aspect, the detector can effect activation of the pump upon detection of a seizure onset. For example, the detector can notify a medical professional of detection of a seizure onset who can in turn activate the pump. Alternatively, the detector can be coupled to the pump so as to activate the pump automatically upon the detection of a seizure onset, with or without an accompanying notification to the medical professional. In some embodiments, the detector can program the pump to set the dose of the radiotracer to be administered to the patient.

In yet another aspect, an imaging system is disclosed that comprises: a patient-specific seizure detector for detecting an onset of a seizure in a patient by classifying at least one feature vector derived from at least one sample of an EEG waveform of the patient as belonging to a seizure class or a non-seizure class, the detector performing the classification by comparison of the feature vector with a measure based on previously-obtained reference EEG waveforms of that patient, and an imaging device for acquiring an image of at least a part of the patient upon detection of a seizure onset.

In some embodiments, the imaging system can further comprise a monitor device for monitoring the EEG waveform of the patient, the detector being coupled to the device for receiving the EEG waveform.

In a related aspect, the detector of the imaging system generates a notification signal, e.g., an alarm, upon detection of the seizure onset. In some embodiments, the notification can be sent to a medical personnel who can activate the imaging device, or delay activation to another time. In some embodiments, the notification can be sent to other caregivers. Further, in some embodiments, the imaging device can be optionally coupled to the detector such that the detector can automatically trigger the imaging device, e.g., via a switching circuit thereof, in response to detection of seizure onset, to acquire an image of the patient. The imaging device can include, without limitation, a SPECT imaging device, or an fMRI device.

In another aspect, the invention provides a system for delivering a diagnostic agent to a patient that comprises: a detector adapted to receive at least one waveform indicative of brain activity of a patient, wherein the detector extracts at least a sample of the waveform and generates a feature vector corresponding to the sample. The detector can comprise a classifier trained on previously-obtained reference waveforms of the patient, the classifier identifying a seizure onset by classifying the feature vector as belonging to a seizure class or a non-seizure class based on comparison with a measure derived from the previously-obtained reference waveforms of the patient. The delivery system can further comprise a device for delivering a diagnostic agent to the patient in response to identification of a seizure onset.

In some embodiments, the delivery system can further comprise a monitor device for generating the waveform data. For example, the monitor device can comprise a non-invasive or an invasive EEG measurement device.

In some embodiments, the detector is coupled to the delivery device so as to activate the delivery device upon identification of a seizure onset to deliver the diagnostic agent to the patient. A variety of delivery devices and/or diagnostic agents can be utilized. By way of example, the delivery device can include a pump for infusion of the diagnostic agent into the patient. In some embodiments, the diagnostic agent can be a radiotracer or a dye.

Further understanding of different aspects of the invention can be obtained by reference to the following detailed description in conjunction with the attached drawings, which are described briefly below.

### BRIEF DESCRIPTION OF THE DRAWINGS

FIG. 1A schematically depicts an arrangement of electrodes distributed symmetrically around the scalp utilized in recording of EEG waveforms,

FIG. 1B shown derivations commonly recorded in bipolar EEG waveform measurements,

FIG. 2 presents an exemplary multi-component EEG waveform having a fundamental frequency of 3 Hz,

FIG. 3A presents an exemplary rhythmic EEG waveform trace,

FIG. 3B presents an exemplary arrhythmic EEG waveform trace,

FIG. 4 presents an exemplary EEG waveform exhibiting suppression,

FIG. 5A depicts an exemplary monomorphic EEG waveform,

FIG. 5B depicts an exemplary polymorphic EEG waveform,

FIG. 6 illustrates commonly used clinical designations of different regions of the head,

FIG. 7 depicts an EEG waveform trace exhibiting a theta rhythm artificially placed in context of normal EEG rhythms,

FIG. 8A presents an EEG waveform trace depicting suppression mu activity following fist-clenching,

FIG. 8B presents an EEG waveform trace illustrating several examples of occipital lambda waves,

FIG. 9A is an exemplary EEG waveform trace exhibiting vertex waves,

FIG. 9B is an exemplary EEG waveform exhibiting high-amplitude bursts of 3-7 Hz waveforms over the central and frontal regions that can be observed in children between ages of 6 months and 6 years during the first stage of sleep,

FIG. 10 presents exemplary EEG waveform traces exhibiting examples of K-complexes,

FIG. 11 presents exemplar EEG waveform traces exhibiting examples of sharp waves, which typically have durations between about 70 to 200 milliseconds,

FIG. 12 presents an exemplary EEG waveform trace exhibiting an example of burst-suppression activity,

FIG. 13 is an exemplary EEG waveform trace showing an example of abnormal, high-amplitude, intermittent 2-3 Hz rhythmic activity on a frontal derivation,

FIG. 14 is an exemplary EEG waveform exhibiting an example of electrocerebral inactivity,

FIG. 15A presents an exemplary EEG waveform trace exhibiting a seizure onset characterized by a paroxysmal 10 Hz burst of sharp and monomorphic waves,

FIG. 15B presents another exemplary EEG waveform trance exhibiting another seizure onset having an activity similar to that shown in FIG. 15A but with less prominent discharges on the frontal derivations,

FIGS. 16A and 16B present exemplary seizure waveforms of two different subjects,

FIG. 17 is an exemplary EEG waveform trace illustrating a high frequency activity associated with muscle artifacts,

FIG. 18 illustrates an exemplary EEG waveform exhibiting a low frequency activity associated with eye blinking and a higher frequency activity associated with eye fluttering,

FIG. 19 present exemplary EEG waveforms exhibiting a mixture of slow, fast, and spike activity resulting from glossokinetic and muscle potentials caused by chewing,

FIG. 20 shows a less than 1-Hz baseline variation in the referential recording of an F₇ EEG electrode,

FIG. 21 presents an exemplary EEG waveform trace exhibiting electrostatically coupled artifacts appearing as high amplitude rhythmic waves,

FIG. 22 is a flow chart depicting various steps in one exemplary embodiment of a method of the invention for detection of seizure onsets,

FIG. 23A schematically illustrates a patient-specific system according to one embodiment of the invention for detecting seizure onset, which employs a spatially independent processing (SIP) architecture,

FIG. 23B schematically illustrates a seizure-onset detection system according to another embodiment of the invention, which employs a spatially dependent processing (SDP) architecture,

FIG. 24A present a sample (epoch) of an exemplary spike-and-slow-wave pattern observed in an EEG waveform,

FIG. 24B is a subband signal obtained by a wavelet decomposition of the waveform of claim 24A, containing the short time-scale “spike” component,

FIG. 24C is another subband signal obtained by a wavelet decomposition of the waveform of FIG. 24A, containing the long time-scale “wave” component,

FIG. 25 is an exemplary iterated filterbank suitable for use in the practice of the invention for performing wavelet decomposition of EEG waveforms,

FIG. 26A illustrates the effective impulse responses of an exemplary implementation of the filterbank of FIG. 25 for producing four subbands that collectively represent EEG activity at time-scales corresponding to frequencies between about 0.5 and 25 Hz,

FIG. 26B illustrates the frequency responses of the filterbank of FIG. 26A,

FIG. 27 graphically illustrates a one-dimensional probability density estimation using kernels,

FIG. 28 illustrates a plurality of exemplary patient-specific training feature vectors that can be utilized by a maximum-likelihood classifier of a seizure detection according to some embodiments of the invention to generate an exemplary decision region,

FIG. 29A graphically shows exemplary estimates of seizure and non-seizure likelihoods constructed by employing training feature vectors and kernel density estimation,

FIG. 29B graphically illustrates an exemplary decision region computed based on the estimates of FIG. 29A,

FIG. 30 depicts a plurality of exemplary patient-specific training feature vectors utilized by a support-vector machine in an embodiment of the invention to determine a decision region,

FIG. 31A graphically presents a linear decision boundary computed by a support-vector machine in an embodiment of the invention based on training feature vectors,

FIG. 31B graphically presents an exemplary non-linear decision boundary computed by a support-vector machine in one embodiment of the invention,

FIG. 32 schematically illustrates a group of EEG derivations, one or more of which can be utilized in identifying a seizure onset in some embodiments of the invention,

FIG. 33 present test EEG waveforms exhibiting electrographic onset of a seizure,

FIG. 34 shows an exemplary training seizure presented to an exemplary detector according to one embodiment of the invention to train the detector to identify the test seizure presented in FIG. 33,

FIGS. 35A-35F present exemplary non-seizure training EEG waveforms that supplement the training seizure of FIG. 34,

FIG. 36 shows an EEG derivation selected by an exemplary seizure detector provided with the training waveforms of FIGS. 34 and 35A-35F,

FIG. 37 graphically presents identification of a seizure event in the test waveform of FIG. 33 by an exemplary detector of the invention trained on seizure and non-seizure EEG waveforms, such as those depicted in FIG. 34 and FIGS. 35A-35F,

FIG. 38 shows test EEG waveforms exhibiting an electrographic seizure,

FIG. 39 shown an exemplary training seizure utilized to train an exemplary seizure detector of the invention to identify the test seizure shown in FIG. 38,

FIG. 40 graphically illustrates detection of the test seizure shown in FIG. 39 by an exemplary trained detector of the invention,

FIG. 41 shows several electrographic seizure onsets suitable for training a seizure detector of the invention,

FIG. 42 shows EEG waveforms exhibiting generalized, periodic discharges occurring between seizure events,

FIG. 43 illustrates exemplary performance of an exemplary seizure detector according to one embodiment of the invention that combines the SIP architecture with maximum-likelihood classifiers,

FIG. 44A-44C exemplary performance metrics for exemplary seizure detectors according to some embodiments of the invention having the SIP architecture and utilizing support-vector machines,

FIG. 45 provides graphs comparing the average detection latency of an exemplary detector that combines the SIP architecture with maximum-likelihood classifiers with that of a similar detector that employs support-vector classifiers,

FIG. 46A presents data corresponding to false-detections declared on test subjects by an exemplary detector that combines the SIP architecture with maximum-likelihood classifier and by an exemplary detector that combines the SIP architecture with support-vector classifiers,

FIG. 46B presents data corresponding to true-detections declared on test subjects for two exemplary detectors, one of which has an SIP architecture with maximum-likelihood classifiers and another has an SIP architecture with support-vector machine classifiers,

FIG. 47 presents graphs indicating performance sensitivity of a detector with the SDP architecture and maximum-likelihood classifiers as a function of several operating parameters,

FIG. 48 presents graphs indicating performance sensitivity of a detector with the SDP architecture and support-vector machine classifiers as a function of several operating parameters,

FIG. 49 presents data corresponding to average detection latency of two exemplary seizure detectors of the invention having the SDP architecture, one with a maximum-likelihood classifier and the other with a support-vector machine classifier,

FIG. 50A presents exemplary test data corresponding to false-detection declared on a plurality of test subjects by two exemplary seizure detectors having the SDP architecture, one with a maximum-likelihood classifier and the other a support-vector machine classifier,

FIG. 50B presents data corresponding to true-detections declared on a plurality of test subjects by the two exemplary seizure detectors generating data presented in FIG. 50A,

FIG. 51A presents exemplary test data obtained in a case study, comparing the performance of exemplary seizure detectors having SIP and SDP architectures with maximum-likelihood classifiers,

FIG. 51B presents exemplary test data obtained in a case study, comparing the performance of exemplary seizure detectors having SIP and SDP architecture with support-vector machine classifiers,

FIG. 52 present test data illustrating the improvement in average detection latency and true-detection rate of an exemplary patient-specific detector as a function of increase in the number of training EEG recordings,

FIG. 53 schematically illustrates a seizure detector in accordance with one embodiment of the invention,

FIG. 54A illustrates an iterated filterbank suitable for use in the exemplary detector of FIG. 53 for wavelet decomposition of EEG waveforms,

FIG. 54B illustrates the first two levels of a polyphase filterbank suitable for use in the exemplary detector of FIG. 53 for wavelet decomposition of EEG waveforms,

FIG. 55 is a flow chart depicting various steps in an exemplary embodiment of method according to the teachings of the invention for detecting onset of alpha waves in a subject,

FIG. 56A presents examples of non-alpha waves training EEG waveforms utilized for training an exemplary detector according to one embodiment of the invention to detect alpha wave onsets,

FIG. 56B presents examples of alpha waves training EEG waveforms utilized for training an exemplary detector according to one embodiment of the invention to detect alpha wave onsets,

FIG. 57 graphically presents detection of alpha waves onset by an exemplary detector according to one embodiment of the invention, which was trained by employing the EEG waveforms such as those shown in FIG. 56A and FIG. 56B,

FIG. 58 presents alpha waves appearing on channels {FP1-F3; FP2-F4} rather than channels {C3-P3; C4-P4},

FIG. 59A shows examples of base line and artifact-contaminated training EEG waveforms utilized to train an exemplary ambulatory seizure detector according to one embodiment of the invention,

FIG. 59B shows an example of a training electrographic seizure utilized to train the exemplary ambulatory seizure detector for which the training non-seizure EEG waveforms of FIG. 59A were employed,

FIG. 60 graphically shows detection of the onset of an epileptiform within 3 seconds with no false detections on the preceding artifacts by an exemplary trained ambulatory seizure detector according to one embodiment of the invention,

FIG. 61 schematically presents an embodiment of a seizure detector according to the teachings of the invention capable of identifying onsets of patient-specific seizures of different types,

FIG. 62 is a flow chart depicting various steps in an exemplary embodiment of a method according to the teachings of the invention for acquiring diagnostic data from a patient in response to detection of seizure onset,

FIG. 63A schematically illustrates an exemplary imaging system in accordance with one embodiment of the invention for obtaining an image of a patient in response to detection of a seizure onset,

FIG. 63B schematically illustrates another embodiment of an imaging system according to the teachings of the invention,

FIG. 64A schematically depicts a system according to one embodiment of the invention for administrating a radiotracer to a patient in response to detection of a seizure onset,

FIG. 64B schematically illustrates an embodiment of an ictal SPECT imaging system according to the teachings of the invention,

FIG. 64C schematically depicts a diagnostic/therapeutic system according to one embodiment for presenting detected seizure EEG waveform(s) as well as reference EEG waveform to a medical professional upon automatic detection of a seizure onset.

FIG. 65 schematically depicts correlating segment of a patient's image with one or more seizure events occurring during at least a portion of a time period in which the image was obtained in accordance with one aspect of the invention,

FIG. 66 is a flow chart depicting various steps in a method according to one aspect of the invention for applying a stimulus to a subject in response to detection of a seizure onset,

FIG. 67 schematically illustrates an exemplary system according to one embodiment of the invention for applying a stimulus to a patient in response to detection of a seizure onset, and

FIG. 68 schematically depicts an exemplary portable vagus nerve stimulator system according to one embodiment of the invention.

### DETAILED DESCRIPTION

The invention pertains generally to automatic detection of selected changes in EEG waveforms of a subject. By way of non-limiting applications, the invention is related to automatic detection of onset of seizures, as well as diagnostic and therapeutic methods and systems related to epilepsy. There are many different types of seizures. The kind of seizure a subject experiences depends on which parts, and how much of the brain is affected by the electrical disturbance that produces seizures. Seizures are typically divided into generalized seizures (absence, atonic, tonic-clonic, myoclonic) and partial (simple and complex) seizures.

Generalized seizures affect both cerebral hemispheres (sides of the brain) from the beginning of the seizure. They produce loss of consciousness, either briefly or for a longer period of time, and are sub-categorized into several major types: generalized tonic clonic; myoclonic; absence; and atonic.

Absence seizures (also called petit mal seizures) are lapses of awareness, sometimes with staring, that begin and end abruptly, typically lasting only a few seconds. There is no warning and no after-effect. Some absence seizures are accompanied by brief myoclonic jerking of the eyelids or facial muscles, or by variable loss of muscle tone. More prolonged attacks may be accompanied by automatisms, which may lead them to be confused with complex partial seizures. However, complex partial seizures last longer, may be preceded by an aura, and are usually marked by some type of confusion following the seizure.

Myoclonic seizures are rapid, brief contractions of bodily muscles, which usually occur at the same time on both sides of the body. Occasionally, they involve one arm or a foot. People usually think of them as sudden jerks or clumsiness. A variant of the experience, common to many people who do not have epilepsy, is the sudden jerk of a foot or limb during sleep.

Atonic seizures produce an abrupt loss of muscle tone. Other names for this type of seizure include drop attacks, astatic or akinetic seizures. They produce head drops, loss of posture, or sudden collapse. Because they are so abrupt, without any warning, and because the people who experience them fall with force, atonic seizures can result in injuries, for example, to the head and face.

Generalized tonic clonic seizures (grand mal seizures) are the best known type of generalized seizure, though not the most common. They begin with stiffening of the limbs (the tonic phase), followed by jerking of the limbs and face (the clonic phase). During the tonic phase, breathing may decrease or cease altogether, producing cyanosis (blueish discoloration) of the lips, nail beds, and face. Breathing typically returns during the clonic (jerking) phase, but it may be irregular. This clonic phase usually lasts less than a minute. Some people experience only the tonic, or stiffening phase of the seizure; others exhibit only the clonic phase or jerking movements; still others may have a tonic-clonic-tonic pattern.

In partial seizures the onset of the electrical disturbance is limited to a specific area of one cerebral hemisphere (side of the brain). Partial seizures are subdivided into simple partial seizures (in which consciousness is retained); and complex partial seizures (in which consciousness is impaired or lost). Partial seizures may spread to cause a generalized seizure, in which case the classification category is partial seizures secondarily generalized.

Partial seizures are the most common type of seizure experienced by people with epilepsy. Virtually any movement, sensory, or emotional symptom can occur as part of a partial seizure, including complex visual or auditory hallucinations. There are two types of partial seizure, simple partial seizures and complex partial seizures.

People who have simple partial seizures do not lose consciousness during the seizure. However, some people, although fully aware of what's going on, find they can't speak or move until the seizure is over. Simple partial seizures include autonomic and mental symptoms and sensory symptoms such as olfaction, audition, or vision, sometimes concomitant with symptoms of experiences such as deja-vu and jamais-vu. Affected individuals remain awake and aware throughout. Sometimes they talk quite normally to other people during the seizure. And they can usually remember exactly what happened to them while it was going on.

Complex partial seizures typically affect a larger area of the brain than simple partial seizures and they affect consciousness. During a complex partial seizure, a person cannot interact normally with other people, is not in control of his movements, speech, or actions; does not know what he is doing; and cannot remember afterwards what happened during the seizure. Although someone experiencing a complex partial seizure may appear to be conscious because he stays on his feet, his eyes are open and he can move about, he is experiencing an altered consciousness, a dreamlike, almost trancelike state. A person may even be able to speak, but the words are unlikely to make sense and he or she will not be able to respond to others in an appropriate way. Although complex partial seizures can affect any area of the brain, they often take place in one or both of the brain's two temporal lobes. Because of this, the condition is sometimes called “temporal lobe epilepsy.”

Epileptic seizures are the outward manifestation of excessive and/or hypersynchronous abnormal activity of neurons in the cerebral cortex. Many types of seizures occur, as described above. The neuromechanism responsible for seizures may include any part of the brain, including but not limited to the amygdala, the hippocampus, the hypothalamus, the parolfactory cortex, the frontal and temporal lobes, and the substantia nigra, a particular portion of the brain considered to be part of neural circuitry referred to as the basal ganglia (See e.g., Depaulis, et al. (1994) Prog. Neurobiology, 42: 33-52).

The methods and systems of the invention can be used to be used to detect, inhibit, reduce, or treat seizures that include, but are not limited to, tonic seizures, tonic-clonic seizures, atypical absence seizures, atonic seizures, myoclonic seizures, clonic seizures, simple partial seizures, complex partial seizures, and secondary generalized seizures.

The terms “patient” and “subject” are employed herein interchangeably and are intended to include generally a living organism, and more preferably a mammal. Examples of subjects include but are not limited to, humans, monkeys, dogs, cats, mice, rates, cows, horses, pigs, goats and sheep.

In many embodiments of the invention described below, one or more waveforms indicative of a patient's brain activity are obtained by performing non-invasive electroencephalogram (EEG) measurements. Although in many preferred embodiments of the invention, non-invasive EEG measurement are employed, in other embodiments, invasive EEG measurement can be utilized for practicing the teachings of the invention. A brief background regarding methodology for acquiring EEG signals and quantitative variables for characterizing them is provided below before discussing various aspects of the invention.

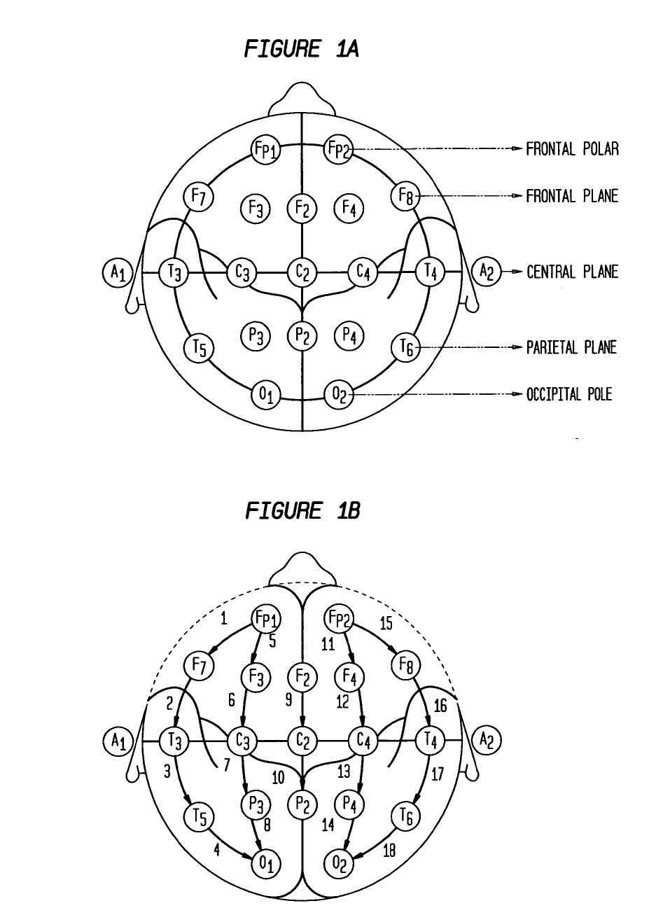

In a typical non-invasive EEG measurement, a plurality of electrodes are employed to monitor and record time-varying electrical potentials at different locations of a subject's scalp, which are generated by millions of cortical neurons. As shown schematically in FIG. 1A a plurality of electrodes can be distributed symmetrically around the scalp to provide temporal and spatial information regarding the brain surface activity. Each electrode responds to an aggregate potential generated by many neurons in the area beneath it. EEG activity of clinical relevance is roughly limited to a frequency band of about 0.5-50 Hz, and that of seizure activity is typically further limited to a frequency band of about 0.5 to about 25 Hz.

Referential as well as bipolar recordings are generally employed for obtaining, and recording, EEG signals. In a referential recording, the electrical potential at each electrode is recorded relative to the potential at either one of the reference electrodes, for example, A1 or A2, as shown in FIG. 1A. Typically, the electrodes from the left-side of the head are cross-referenced to A2 while those from the right-side of the head are cross-referenced to A1. This scheme ensures that electrodes for each side of the head measure cerebral activity relative to a reference that is not significantly affected by cerebral activity within their areas of coverage. Any electrode can be used as a reference for the others, but commonly used references, besides A1 and A2 are Cz and an average of all electrodes. In bipolar recording, difference potentials between pairs of adjacent electrodes are measured. Such a pair-wise potential difference is also known as a derivation. FIG. 1B schematically shows longitudinal derivations most commonly recorded in EEG measurements. The electrical potential of the electrode at the tip of an arrow is subtracted from the potential of the electrode at the tail of the arrow.

An advantage of referential recordings is that a change or abnormality can be clearly observed since the absolute electrode potentials, rather than their differences, are the quantities that are recorded. A disadvantage of referential recordings is that they can be susceptible to common-mode noise as well as contamination of the reference electrode by artifact activity, or by the brain activity that is being analyzed (active reference). Once the reference electrode is contaminated it becomes difficult to interpret the activity on electrodes measured relative to it.

Bipolar recordings overcome common-mode noise by subtracting potentials on contiguous electrodes. The consequence of this operation is a slight attenuation of changes or abnormalities observed in the EEG. An extreme case occurs when a derivations records a zero signal due to cerebral activity that equally affects its electrodes.

In many embodiments described below, bipolar EEG signals are employed in predicting onset of a seizure as their lower susceptibility to artifacts can outweigh typical slight attenuation in signals. It should, however, be understood that the teachings of the invention can also be practiced by utilizing referential recordings. In addition, in some embodiments of the invention, invasive EEG recordings can be employed for detecting onset of a seizure. As is known in the art, an invasive EEG recording is made by utilizing electrodes that are in direct contact with the brain surface. Such invasive recordings are commonly known as electrocorticograms (ECoG). EcoG recordings can provide a better spatial resolution than non-invasive recordings as each electrode responds to the activity of a far smaller number of cortical neurons. ECoG can also be less susceptible to signal attenuation and artifacts. However, non-invasive EEG waveforms can be more readily obtained.

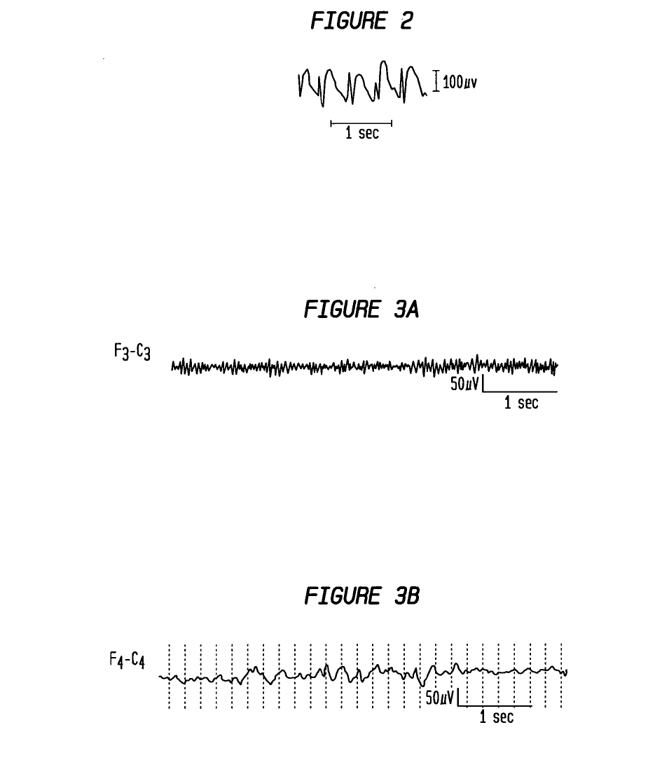

EEG activity can be characterized in terms of several quantitative and qualitative variables that need to be considered in the context of a patient's age and state of consciousness. Some typically employed variable include: fundamental frequency, amplitude, morphology, localization and reactivity. The fundamental frequency of an EEG waveform, typically measured in Hertz (Hz), refers to the rate at which the waveform is repeated over a period of a second. The waveform can have an arbitrary shape and any number of subcomponents, all that matters is rate at which the unit as a whole repeats in the span of a second. For instance, the multi-component waveform shown in FIG. 2 has a fundamental frequency of 3 Hz. An EEG waveform with a constant, stable fundamental frequency, such as that shown in FIG. 3A, is called rhythmic. In contrast, a waveform lacking such a constant, stable fundamental frequency, such as that shown in FIG. 3B is called arrhythmic.

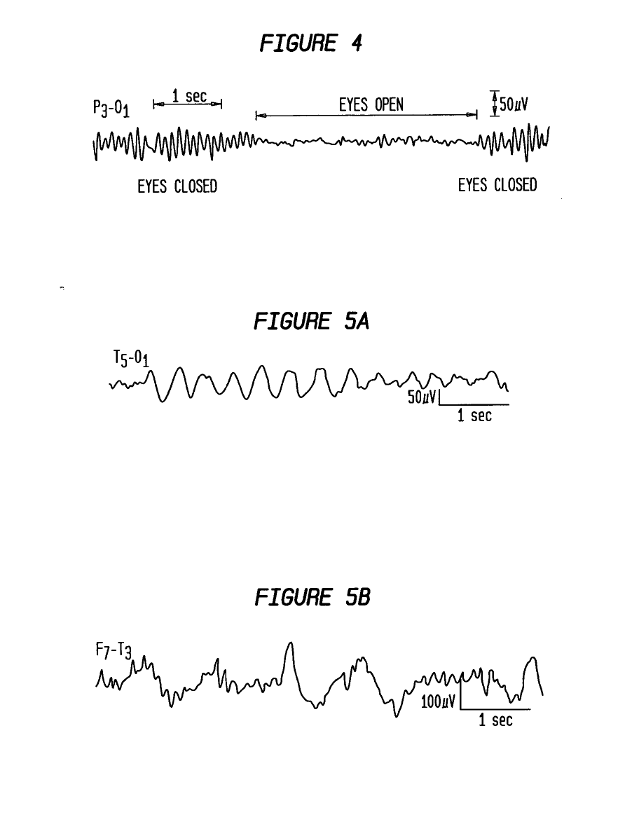

The amplitude of a waveform in an EEG trace refers to its peak voltage, which is typically on the order of microvolts. For example, the waveforms in the exemplary EEG trace of FIG. 3A have amplitudes smaller than 75 micro-volts (μV), and those in the trace of FIG. 2 have an amplitude of approximately 100 μV. An EEG waveform demonstrating a sudden or gradual reduction in amplitude, such as that illustrated in FIG. 4, is said to exhibit suppression or depression.

The morphology of an EEG waveform describes its observed shape, which is a function of the amplitude and fundamental frequency of its constituent components. An EEG waveform that is composed of a single component is called monomorphic, and one that is composed of several different components is called polymorphic. Examples of these two different morphologies are shown in FIGS. 5A and 5B, respectively.

EEG traces that consist of two or more waveforms, each with possibly different morphologies, are called complexes. An example of a commonly observed abnormal complex is the “spike-and-slow-wave complex” shown in FIG. 2. As its name implies, a spike-and-slow-wave complex is composed of a broad, slow wave and a transient spike.

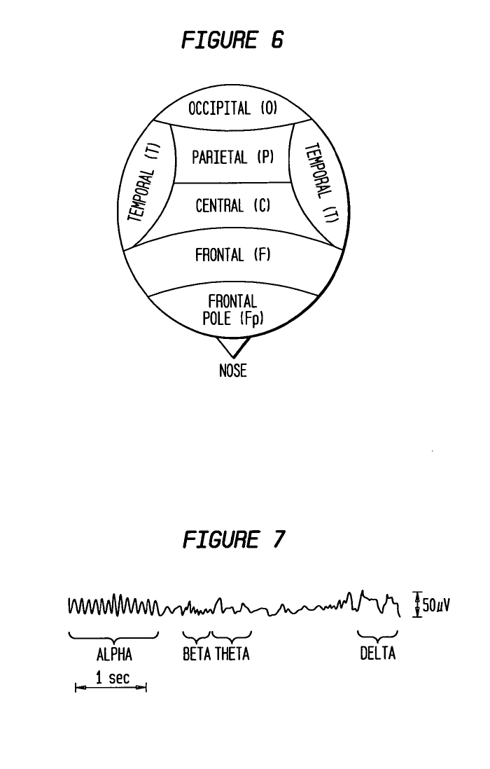

The localization of EEG activity refers to the distribution of the activity over the subject's head. EEG activity observed only in a limited region of the head is called focal while activity observed in all regions is called generalized. Furthermore, EEG activity exhibiting equal fundamental frequency, amplitude, and morphology on the left and right sides of the head is referred to as symmetric, otherwise it is referred to as asymmetric. The clinical designations for different regions of the head are shown in FIG. 6.

The reactivity of EEG waveforms refers to the degree of change in anyone of the preceding variables as a result of a stimulus. For instance, FIG. 4 shows the suppression of 10-Hz occipital activity upon opening of the eyes.

Normal EEG activity is any activity that qualitatively and quantitatively appears mostly in the EEG of subjects not affected by any disease. The following is a description of well-documented normal EEG activity in adults and children.

The alpha rhythm is an EEG activity, with frequencies between about 8-13 Hz, which is prominent in the occipital regions of normal, relaxed adults whose eyes are closed. Alpha activity is attenuated by opening of the eyes, increased vigilance, or heightened awareness as exhibited in the exemplary alpha waveform shown in FIG. 4. A mixture of the alpha rhythm with other rhythms results in alpha variants, which have different morphology but exhibit the same reactivity and localization.

The frequency of alpha rhythms in children gradually increases towards the rate observed in adults over the course of their development. The alpha rhythm may be as slow as 3 Hz at the age of two months and as fast as 7 Hz at the age of one year. Furthermore, the amplitude of alpha rhythms in children steadily increases until the age of one year, and then declines towards the 10 μV-50 μV level observed in adults.

The beta rhythm is an EEG activity, with a frequency exceeding about 13 Hz, which is most prominently observed in the frontal and central regions in adults, but may also be generalized. Alertness and vigilance promotes the onset of beta activity, while voluntary movement results in its suppression. FIG. 3A illustrates rhythmic beta activity recorded from the F₃-C₃ central derivation. The beta rhythm also shows a gradual, age-related increase in frequency for children.

The theta rhythm is an EEG activity with a frequency in a range of about 4 to 7 Hz. This activity is abnormal in awake adults, but commonly observed in sleep and in children below the age of 13 years. Theta activity is asymmetric since it is predominantly observed in the central, temporal, and parietal regions of the left side of the head. FIG. 7 shows the theta rhythm artificially placed in context of other normal EEG rhythms.

The delta rhythm exhibits a frequency below about 4 Hz and amplitudes that exceed those of all other rhythms. It is most prominent frontally in adults and posteriorly in children in the third and fourth stages of sleep. FIG. 7 shows the delta rhythm artificially placed in context of other normal EEG rhythms.

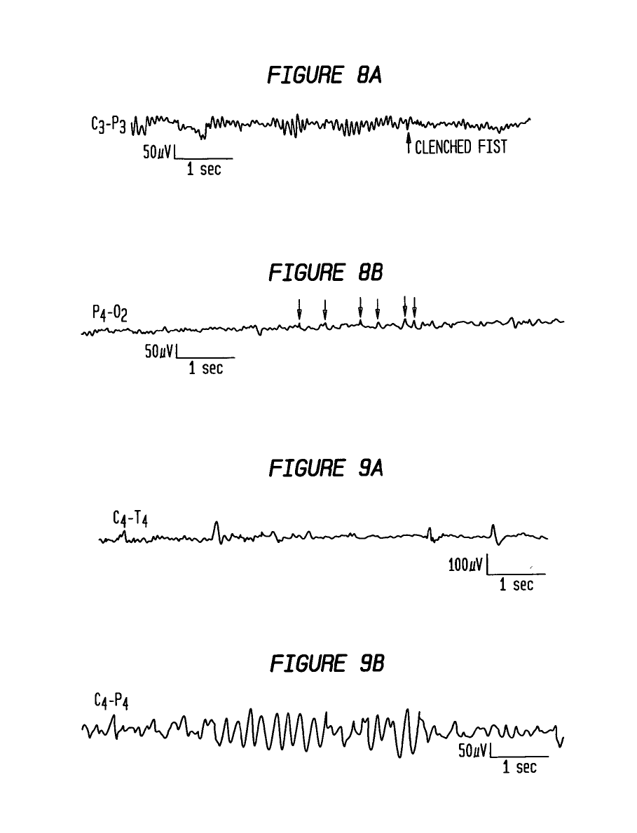

The mu rhythm refers to an EEG activity with a frequency between about 7 to 11 Hz that is most prominently observed in the central region. Mu activity is suppressed by movement (e.g., fist clenching), imagined movement, or tactile stimulation; in contrast, it is enhanced by immobility and decreased attention. While the frequency range of mu and alpha rhythms overlap, mu rhythms are differentiated by their localization, arch-like morphology, and reactivity. FIG. 8A shows the suppression mu activity following fist-clenching in an exemplary EEG waveform.

Lambda waves are transient sharp waves lasting approximately 0.25 seconds that occur in the occipital region whenever an adult scans a visual field with horizontal eye movement. Lambda waves are not seen when the eyes are closed, or opened in dark settings. Lambda waves exhibit the same localization and reactivity in children as in adults. FIG. 8B illustrates several examples of occipital lambda waves.

Sleep-spindles, K-complexes, and vertex waves are unique waveforms observed only during the four different stages of sleep. The salient characteristics of these waveforms and the four stages of sleep in both adult and children are discussed below. In the first stage of adult sleep, alpha activity is typically attenuated while theta activity becomes more prominent in the temporal regions. Further, a series of positive occipital sharp transients may he observed. Deeper into the first stage of sleep, vertex waves, which are the sharp waves exhibited by the exemplary waveform shown FIG. 9A, begin to appear centrally. For children between the ages of 6 months and 6 years, the first stage of sleep can be accompanied by high-amplitude bursts of 3 to-5-Hz waveforms over the central and frontal regions that can last between several seconds and several minutes. This activity, which is illustrated in an exemplary waveform shown in FIG. 9B, can be easily mistaken for a seizure without knowledge of the child's state of consciousness.

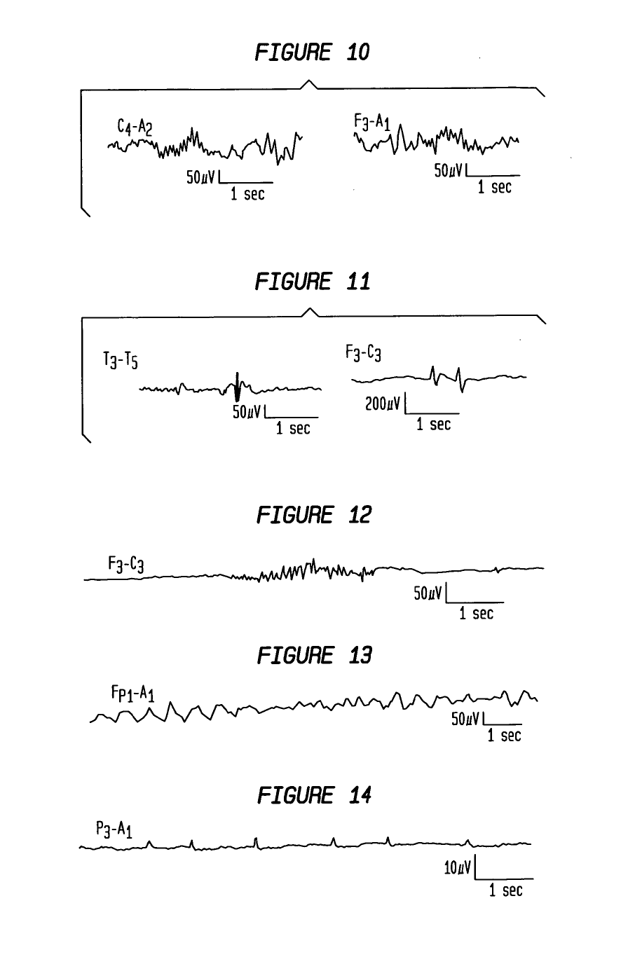

In the second stage of adult sleep, alpha activity is virtually absent while theta activity and vertex waves are more prominent, and rhythmic bursts called sleep-spindles with frequencies around 14 Hz appear centrally. Also common in the second stage of sleep are k-complexes, which are sharp, slow transients immediately followed by sleep-spindles. Examples of these waveforms are shown in FIG. 10.

Sleep spindles are absent from the EEG of children until sometime between 6 weeks and 2 months of age. When they first begin to appear in the second stage of sleep, the sleep spindles of young children exhibit sharper negative peaks than those of adults. K-complexes remain absent from the second stage of sleep in children until sometime between 3-4 months of age.

In the third stage of sleep, delta activity and slow frontal transients become increasingly prominent while sleep spindles and K-complexes are observed to a lesser degree. The fourth stage of sleep extends the activity of the third stage with sleep spindles slowing down to a frequency of 10 Hz.

EEG activity can be generally classified into normal and abnormal activity. Abnormal EEG activity can be considered as any activity that is prevalent in the EEG of groups of people with neurological or other disease complaints, and absent from that of normal individuals. Abnormal EEG may be an unusual waveform as well as the absence or deviation of normal EEG from well-documented limits on frequency, amplitude, morphology, localization, and reactivity. For instance, an EEG recording exhibiting an absence of or a change in the nominal frequency and amplitude of sleep spindles can be considered abnormal. By way of further elucidation, the several abnormal EEG waveforms that are commonly observed in the EEG of patient groups are discussed below. For patients affected by epilepsy, these abnormalities are routinely observed during interictal periods, that is, periods between seizure episodes. However, they do not necessarily result in the clinical behavior observed during a seizure or match its electrographic signature.

By way of example, spike waves are transients with pointed peaks exhibiting durations typically between about 20 to 70 milliseconds. Sharp waves are similar to spike waves, but exhibit longer durations typically between 70-200 milliseconds, as shown in exemplary waveforms of FIG. 11. A spike-and-slow-wave complex is a spike followed by a longer duration wave, as shown in exemplary waveform of FIG. 2. Multiple spikes may precede the slower wave and the entire complex may be repeated at rates of 2.5-6 Hz with intervening periods of quiescence of various durations. A sharp-and-slow-wave complex is identical to the spike-and-slow-wave complex except that a sharp wave precedes the slower, broader wave and the complex is repeated at rates between 1-2 Hz.

Periodic discharges refer to time-limited bursts that are repeated at a certain rate. These bursts may exhibit a variety of durations, frequencies, amplitudes, morphologies, and localizations. An example of a periodic discharge is burst-suppression activity, which is a discharge of theta or delta frequency waveforms with long intervening periods of very low-amplitude waves. FIG. 12 provides an example of burst-suppression activity.

Rhythmic hypersynchrony refers to rhythmic activity emerging from a quiescent back-ground and exhibiting unusual frequency, amplitude, morphology and localization of any degree. The rhythmic activity may either be continuous or intermittent. FIG. 13 shows an example of abnormal, high-amplitude, intermittent 2 to-3-Hz rhythmic activity on a frontal derivation.

Electrocerebral inactivity refers to a variable length period not caused by instrumental or physiological artifacts that exhibits extreme attenuation of the EEG relative to a patient-specific baseline, as shown in exemplary waveform of FIG. 14. To appreciate the reduced amplitude of this trace, note that a 10 μV scale, rather than a 50 μV scale is used to present the waveform trace of FIG. 14. Furthermore, the transients in the waveform of FIG. 14 are not of cerebral origin, they are the result of electrocardiographic artifacts.

Seizures are abnormal, continuous neuronal discharges with clinical correlates that can include an involuntary alteration in behavior, movement, sensation, or consciousness. Seizures without clinical correlates are called subclinical seizures. The electrographic signature of a seizure can be composed of a continuous discharge of variable amplitude and frequency polymorphic waveforms, spike and sharp wave complexes, rhythmic hypersynchrony, or electrocerebral inactivity observed over a duration longer than the average duration of these abnormalities during interictal periods. Furthermore, the abnormalities observed during interictal periods need not necessarily be those that compose the seizure's electrographic signature.

The electrographic signature of a specific seizure type for a given patient is usually stereotypical and distinguishable from their non-seizure activity. A patient can exhibit more than one type of seizure, however each type will have a stereotypical electrographic and clinical manifestation. The seizures of two different patients can exhibit very distinct morphology and localization. Moreover, the characteristics of one patient's non-seizure activity can resemble the seizure activity of another. As discussed in more detail below, the methods and systems of the invention provide patient-specific seizure onset detection, which advantageously minimizes, and preferably eliminates, false positive seizure indication that generally plagues conventional generic (not specific to a given patient) seizure detectors.

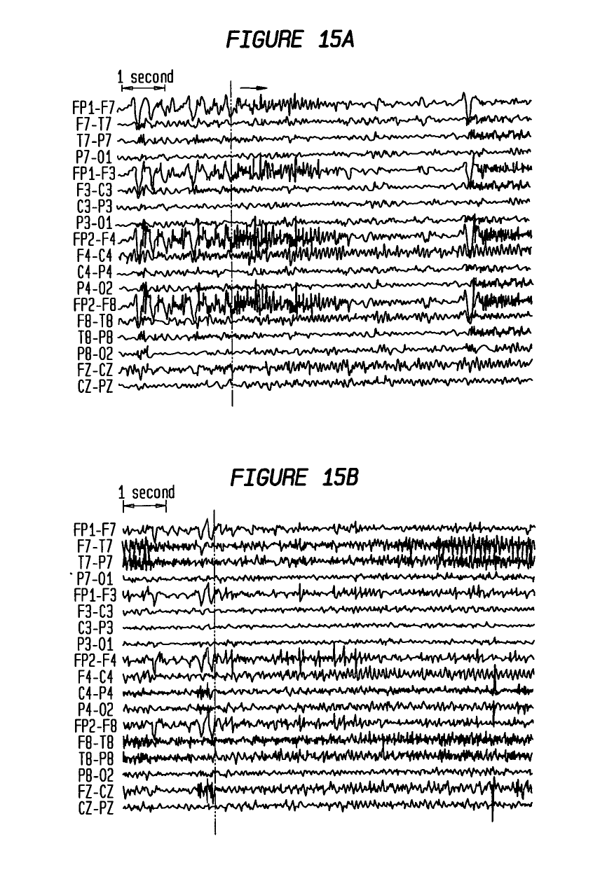

By way of example, FIGS. 15A and 15B illustrate the degree of similarity between two seizure onsets from the same subject. The first seizure onset, shown in the waveforms of FIG. 15A after the dashed line, is characterized by a paroxysmal 10-Hz burst of sharp and monomorphic waves localized primarily to the central derivations {Fz-Cz; Cz-Pz}; the right frontocentral derivations {FP₂-F₁; F₄-C4}, and the right frontal derivations {FP₂-F₈ F₈-T₈; T₈-P₈}. The second seizure onset, shown in the waveforms of FIG. 15B, matches the activity of the first except for less prominent discharges on the frontal derivations {FP₁-F₇; FP₁-F₃; FP₂-F4; FP₂-F₈}.

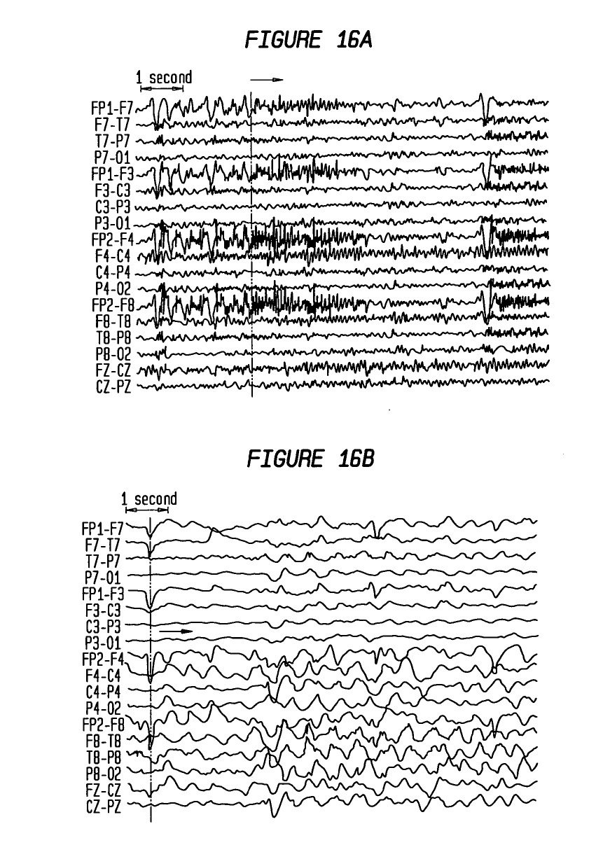

FIGS. 16A and 16B present exemplary seizure waveforms of two different subjects, illustrating the variability in morphology of seizure onset waveforms in different subjects. The seizure onset waveforms depicted in FIG. 16A is characterized by a paroxysmal 10-Hz burst of sharp and monomorphic waves while those depicted in FIG. 16B exhibit a higher-amplitude, paroxysmal 2-Hz burst of monomorphic waves. Coincidentally, the seizure onsets from both subjects localize to the same derivations.

Any electrical activity in EEG that is not of cerebral origin is labeled as an artifact. Artifacts of physiological origin may result, for example, from muscle potentials, electrocardiographic potentials, eye movement potentials, glossokinetic (derived from the tongue) potentials, and skin potentials. Artifacts of nonphysiological origin result primarily from malfunctioning electrodes and electromagnetic interference. Learning the characteristics of these artifacts are generally needed for both an electroencephalographer and an automated seizure detector, since artifacts are prevalent in EEG and can be easily confused with seizure activity.

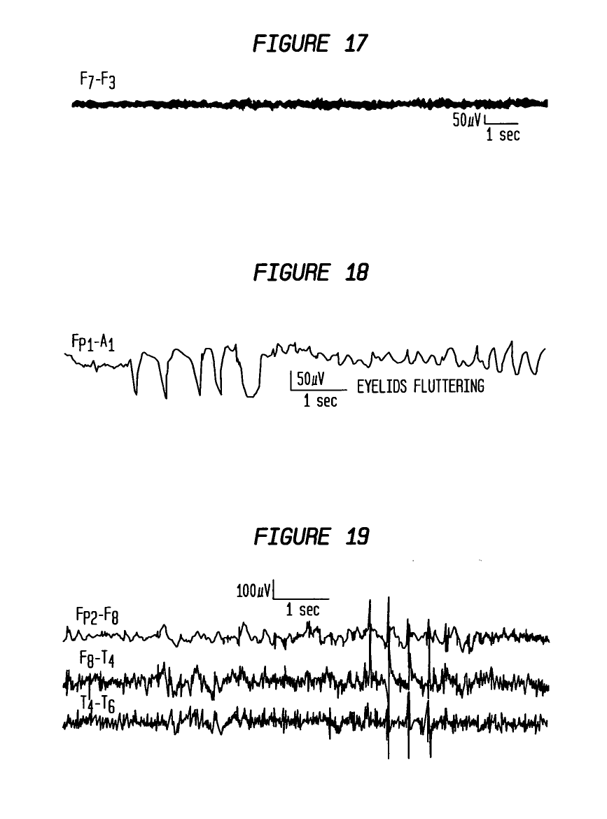

Artifacts caused by muscle potentials are very common in EEG recordings. They typically appear as high-frequency bursts in the frontal and temporal electrodes of a bipolar recording, and in all electrodes of a referential recording that uses the ear, chin, or mandible as a reference. Although muscle artifacts cannot be completely eliminated, they can be attenuated with the use of a high frequency filter that limits the EEG bandwidth to about 35-Hz activity. However, a risk associated with this approach is that highly filtered muscle activity may be mistaken for normal beta activity. By way of example, FIG. 17 illustrates the high frequency activity associated with muscle artifacts.

Eye movement, eye blinking, and eyelid fluttering can give rise to artifacts resembling transient or rhythmic EEG slow waves. These artifacts appear most prominently in the frontal channels of both bipolar and referential recordings, and can possibly be distinguished from EEG activity of frontal cerebral origin by the addition of electrodes around each eye. However, the extra electrodes are not often used in clinical practice. A mixture of eye movement and electrocardiographic artifacts can result in rhythmic frontal activity with sharp and slow components. By way of example, FIG. 18 illustrates an EEG waveform exhibiting the low frequency activity associated with eye blinking and the higher frequency activity associated with eye fluttering.

Artifacts generated by glossokinetic potentials refer to artifacts generated by movement of the tongue. These artifacts can appear as single rhythmic slow waves in the temporal regions and can he recognized by the addition of electrodes near the mouth. Chewing and sucking movements mix artifacts generated by muscle potentials and glossokinetic potentials, and can be identified by the addition of electrodes near the jaw. Finally, hiccups and sobbing can generate glossokinetic potentials that may appear in EEG as abnormal spike-and-wave discharges. FIG. 19 shows exemplary waveforms exhibiting a mixture of slow, fast, and spike activity resulting from glossokinetic and muscle potentials caused by chewing.

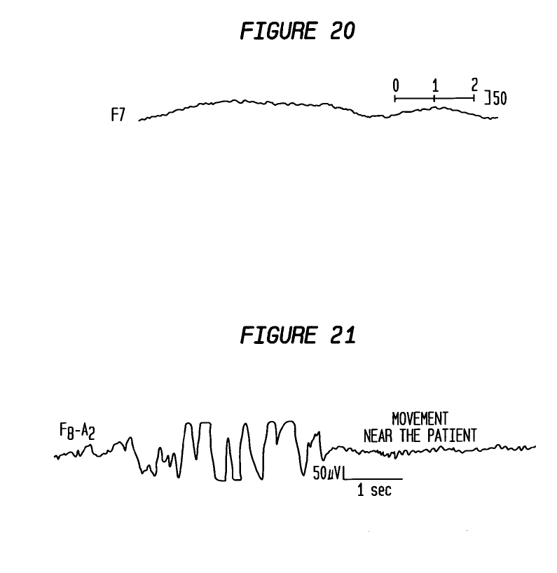

Changes in skin potential produce low frequency baseline changes in the EEG. The potential of skin may change as a result of the electrical potential generated by active sweat glands, or because of sweat-related changes in electrolyte concentration between the skin and the EEG electrodes. FIG. 20 shows a less than 1-Hz baseline variation in the referential recording of an F₇ electrode displayed on a 2 second 50 μV scale.

Electrodes that are poorly coupled mechanically or electrically to the skin can produce artifacts resembling EEG sharp waves, spike waves, or slow waves. Movement of the wires connecting electrodes to the EEG instrument can simulate slow, rhythmic EEG activity with a frequency matching the movement of the wires.

Electromagnetic interference that is coupled electrostatically or inductively to recording electrodes can mask the underlying EEG activity. An example of this type of interference is 60-Hz and higher frequency radiation from surrounding electronic and radio equipment. Furthermore, the movement of personnel around the wires of EEG electrodes can generate electrostatically coupled artifacts that can appear as high amplitude rhythmic waves, as shown in exemplary waveform of FIG. 21.

Another type of artifacts comprise electrographic artifacts that are produced by the electrical activity of the heart. They resemble attenuated periodic sharp waves in both referential and bipolar recordings.

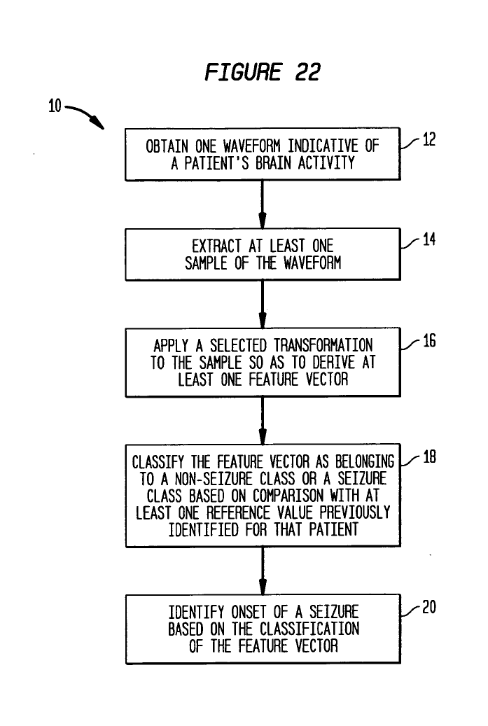

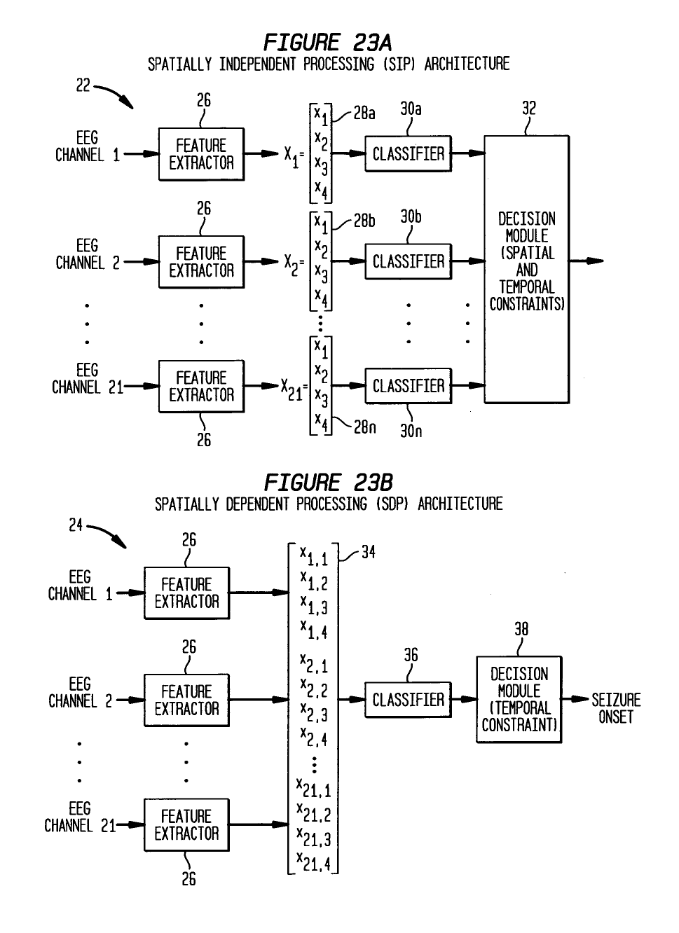

In one aspect, the present invention provides a method of patient-specific detection of seizure onset. With reference to a flow chart 10 of FIG. 22, in one exemplary embodiment, in a step 12, a waveform channel of the patient's brain activity is acquired. The waveform can be, for example, a non-invasive EEG waveform that provides information regarding neural activity in a portion of the patient's brain in a manner described above. In other embodiments, invasive EEG waveforms can be employed. In step 14, one or more samples of the acquired waveform are extracted. The sample can correspond to a selected temporal portion (epoch) of the waveform. For example, one or more two-second portions of the waveform can be sampled. The temporal duration of the extracted sample is not limited to any of the specific values recited herein, and in fact can have any value suitable for a particular application. For example, the extracted sample or samples (herein also referred to as epochs) can have temporal durations in a range of about 1 second to about 5 seconds.

In step 16, a selected transformation is applied to the sampled waveform so as to derive at least one feature vector that includes information regarding the morphology of the waveform sample. A feature vector as used herein refers to one or more values that quantitatively provide information regarding the morphology of the waveform sample. In many embodiments, these values indicate the energy (i.e., signal strength) associated with one or more transform waveforms obtained by applying the selected transformation to the sampled EEG waveform. The energy (signal strength) associated with a transform waveform can be evaluated, for example, by integrating (or summing when the waveform is represented by digital values) the transform waveform's signal amplitudes. For example, the signal strength of a digitized transform waveform can be computed by summing the absolute values of the signal amplitudes at a plurality of discrete points representing that waveform. In many embodiments, the feature vector values represent a function of the energy contained within the transform waveforms, rather than the energy itself, so as to provide a more robust indicator of the morphology of the EEG sampled waveform. Such a function can be any suitable linear or non-linear function. For example, in the embodiments described below, this function is selected to be logarithmic function. However, other functions, such as, a square root function, can also be employed.

The transformation applied to the sampled waveform for generating the feature vector(s) can be, for example, a time-frequency transformation. Such a time-frequency transformation can decompose the sampled waveform into a plurality of signal subbands, each of which contains the EEG sample waveform's components within a selected frequency bandwidth. The frequency bands associated with the signal subbands are preferably selected to be noncongruent, that is, they are selected to be offset from one another (with some degree of overlap or with no overlap). In addition, the frequency bands can have different frequency widths. By way of example, the sample waveform can be decomposed into one or more subband signals by way of analyzing the sample waveform at one or more time-frequency scales defined by the contraction or dilation of a chosen wavelet. In such a case, the feature vector can represent a function of energy contained in the subband signals, as discussed in more detail below.

In step 18, the feature vector is classified as belonging to a seizure class or a non-seizure class based on comparison with at least one reference value previously identified for that patient. The non-seizure class can represent normal as well as artifact-contaminated EEG activity observed in different states of consciousness while the seizure class can present EEG activity observed during seizure onset. The seizure class can include a plurality of seizure types (seizure sub-classes), each representative of seizure onset EEG activity associated with a particular type of seizure. In some embodiments, the feature vector can be classified as not only belonging to a seizure class but can also be assigned to one of the sub-classes whose union provides the seizure class for that patient. The reference value can represent one or more decision boundaries obtained, for example, from support vectors identified based on reference feature vectors obtained from previously-acquired non-seizure and seizure waveforms of the patient. Alternatively, a probabilistic algorithm (e.g., a maximum likelihood algorithm) can be employed to determine the probability that the feature vector associated with the EEG sampled waveform belongs to the seizure class. In other words, both a probabilistic methodology or a determinative methodology can be employed to classify the feature vector generated from the sampled waveform, as discussed in more detail below.

With continued reference to FIG. 22, in step 20, an onset of a seizure of the patient is identified based on the classification of the feature vector(s). In many embodiments, a seizure onset is declared if feature vectors obtained from two or more consecutive waveform samples are classified as belonging to the seizure class.

The embodiments described below further elucidate the methods and systems of the invention. For example, FIG. 23A schematically illustrates a system 22 according to one embodiment of the invention, herein referred to as having a spatially independent processing architecture (SIP), for detecting onset of seizures in a patient while FIG. 23B schematically depicts a seizure-onset detection system 24 according to another embodiment of the invention, herein referred to as having a spatially dependent processing (SDP) architecture. Both systems include a plurality of feature extractors 26 that receive signals corresponding to a plurality of EEG waveform channels (invasive or non-invasive) corresponding to the patient's brain activity. More specifically, in these exemplary embodiments, a two-second epoch from each of twenty-one bipolar EEG derivations is individually passed through one of the feature extractors. Each feature extractor computes four feature values characterizing the amplitude, fundamental frequency and morphology of its associated waveforms.

In the SIP architecture (system 22), the four features extracted from each derivation are assembled into a distinct feature vector (e.g., feature vectors 28a, 28b, . . . , 28n, herein collectively referred to as feature vectors 28) to be assigned to a seizure or a non-seizure class independently of the other derivations. More specifically, the system 22 includes a plurality of classifiers 30a, 30b, . . . , 30n (herein collectively referred to as classifiers 30), each of which receives the feature vector generated by one of the feature extractors. Each classifier is trained on reference feature vectors generated based on previously-obtained EEG waveforms of the patient corresponding to the same derivation as that received by the feature extractor coupled to that classifier, as discussed in more detail below. A decision module 32 declares a final decision regarding the onset of a seizure by examining all of the feature vector classifications in the context of temporal and patient-specific spatial localization constraints, as discussed in more detail below.

In the SDP architecture (system 24), the feature values extracted from all derivations are grouped into a composite feature vector 34 that captures interdependencies that may exist between derivations. A classifier 36 that is trained on EEG waveforms from all derivations then assigns the composite feature vector 34 to either the seizure or the non-seizure class. A decision module 38 in communication with the classifier 36 then declares onset of a seizure if the classification satisfies pre-defined temporal constraints, such as those discussed below. Although the decision module 38 is shown as separate from the classifier, in many embodiments, it is incorporated within the classifier. In other words, the classifier not only classifies the feature vector but it also declares a seizure onset based on that classification.

The above exemplary SIP and SDP architectures differ primarily in the stage in which patient-specific spatial localization constraints are captured or enforced. In the case of the SIP architecture, localization constraints are imposed using explicit rules in the final element of the detector. This permits independent classification of activity on each derivation in a low dimensional feature space. In contrast, the SDP architecture expresses spatial constraints through the elements of a composite feature vector summarizing interrelations between derivations. While this obviates the need to explicitly enforce localization constraints, it hides from the user which derivations are being used for detection; and causes classification to take place in a higher dimensional feature space.

The following sections describe various computational elements employed in the above exemplary seizure onset detectors. In particular, the section under the heading “Feature Extraction” describes how EEG waveforms are analyzed in order to extract features characterizing their amplitude, fundamental frequency, and morphology for constructing the feature vectors. The section under the heading “Classification” describes how the class membership of feature vectors under both architectures is determined by employing patient-specific and non-specific training examples. Further, the section under the heading “Spatial and Temporal Constraints” outlines the temporal and patient-specific localization constraints used in the SIP architecture to determine whether or not classified feature vectors are indicative of seizure onset. Some patient-specific training examples are discussed in the section under the heading “Training.”

### Feature Extraction

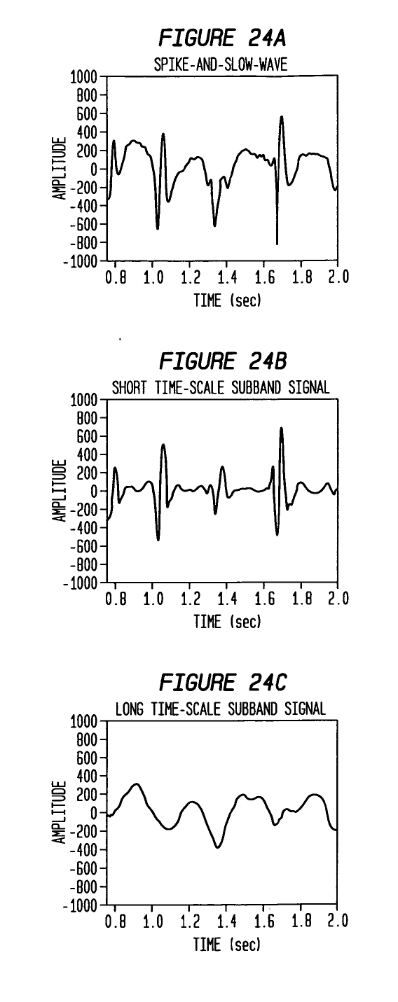

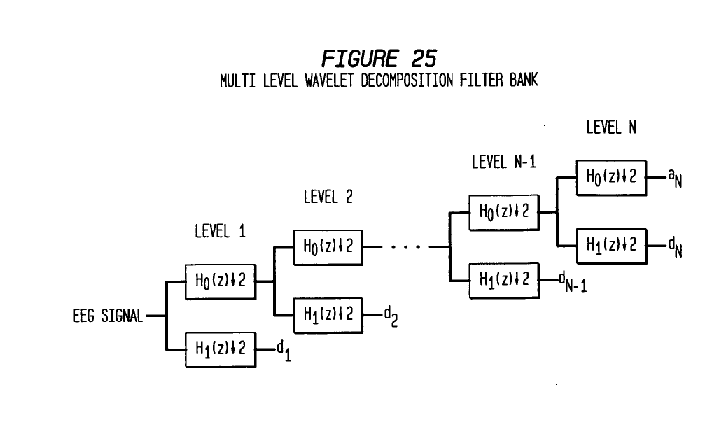

In many embodiments, the samples (epochs) of EEG waveforms are decomposed in a plurality of wavelets to obtain quantities corresponding to amplitude, fundamental frequency and morphology of the waveforms. These quantities can be employed as high-fidelity indicators for discriminating between normal and seizure-onset EEG waveforms. For example, a multi-level wavelet decomposition of an EEG waveform can be employed to extract subband signals containing components contributing to the waveform morphology at specific timescales. For instance, a spike-and-slow-wave pattern (shown in FIG. 24A) can be decomposed into a subband signal containing the short time-scale (high-frequency) “spike” component (FIG. 24B), and another subband signal containing the long time-scale (low-frequency) “wave” component, illustrated in FIG. 24C. A Fourier analysis of the same pattern, rather than a wavelet analysis, would be less sensitive to the short time-scale “spike” component because it provides a description of a signal's global regularities, rather than its local, singular irregularities or non-stationarities. More generally, the wavelet transform is better suited for analyzing non-stationary signals like the EEG in comparison to the Fourier transform, which assumes signal stationarity.

In some embodiments, the subband signals of a multi-level wavelet decomposition can be computed by passing the EEG signal through an iterated filterbank structure linked by downsampling operations (↓2), as shown schematically in FIG. 25. The time-scale or frequency of activity captured by a particular subband signal is predetermined by the iteration level producing it and the choice of analysis filters H₁(z) and H₀(z). Generally, the time-scale re-solved by a subband signal increases the higher its iteration level, which is equivalent to the frequency of the resolved activity decreasing.

By way of example, H₁(z) and H₀(z) can be chosen to be the filters associated with the fourth member of the Daubechies wavelet family. These filters are only four taps long and exhibit a maximally flat response in their passband as well as little spectral leakage in their stophands. Furthermore, in many embodiments, only the subband signals {d₄, d₅, d₆, d₇} are computed because they can collectively faithfully represent activity at time-scales corresponding to frequencies between 0.5-25 Hz; which is a frequency band known to capture seizure onsets of various electrographic manifestations. The remaining subband signals primarily resolve activity of no substantial clinical relevance. For example, the subband signal {a₇} captures slow baseline variations, such as those caused by sweating, while the subband signals {d₁, d₂, d₃} capture high frequency artifacts similar to those resulting from muscular contractions.

To better appreciate the time-scales or frequencies captured within the subband signals {d₄, d₅, d₆, d₇}, one can examine the overall impulse or frequency response of the cascade of filters between the input and each of the output subband signals. The frequency response illustrates the frequencies that will pass through the cascade of filters to appear in a given subband signal. The impulse response highlights the time-scale or duration of activity to which the cascade of filters is most sensitive, consequently appearing in the output subband signal.

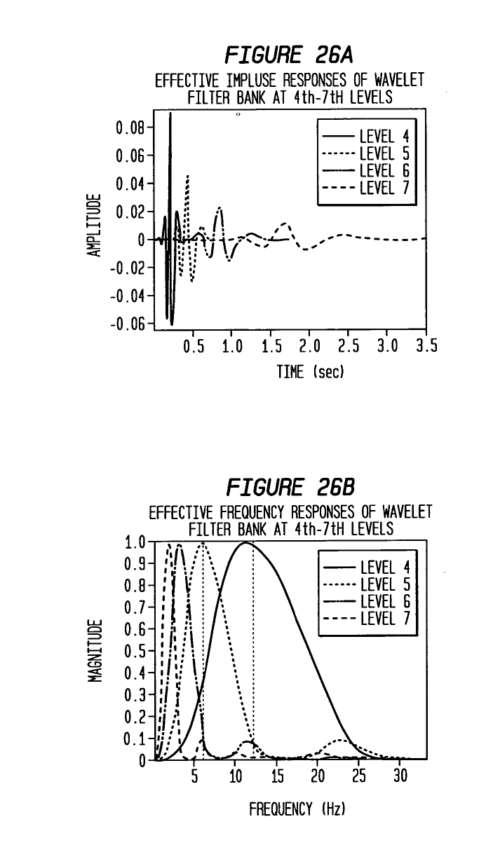

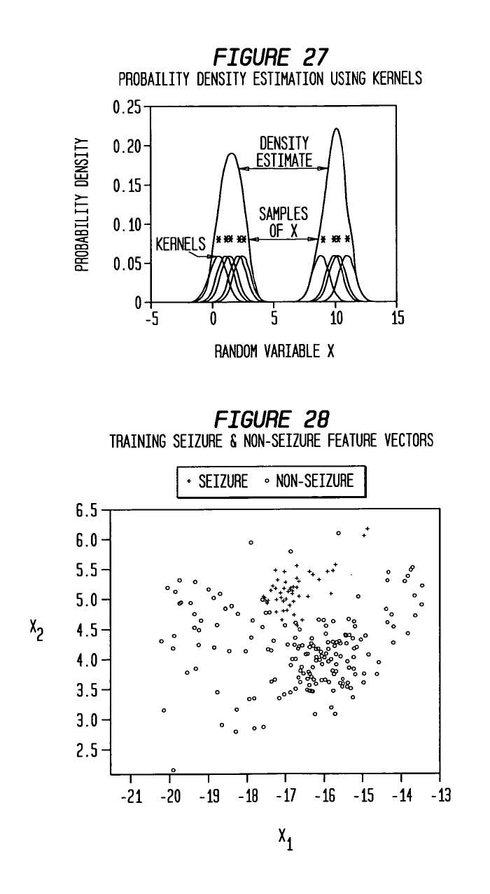

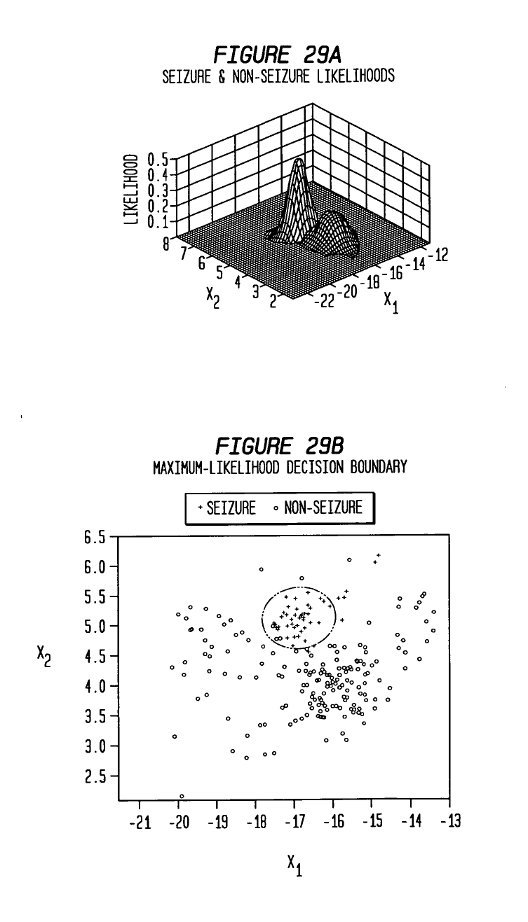

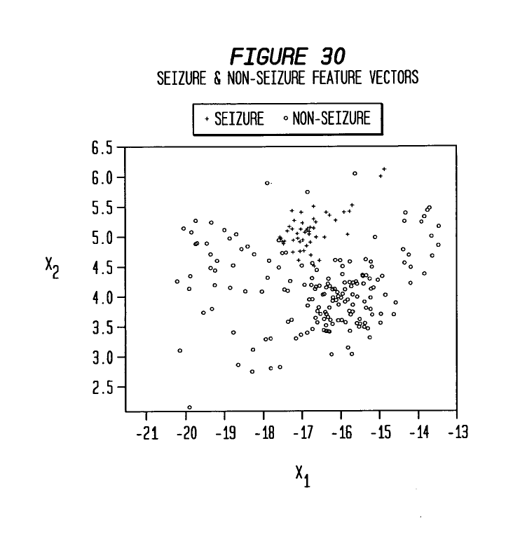

FIGS. 26A and 26B present, respectively, the overall impulse and frequency responses that produce each of the subband signals {d₄, d₅, d₆, d₇}. The impulse responses are progressively stretched for higher level subband signals so that activity of longer time-scales can be represented. This is equivalent to the observed decrease in center frequency and bandwidth of frequency responses associated with filter cascades producing higher level subband signals. In this example, the frequency bandwidth associated with each level (e.g., characterized by full width at half maximum of the response) is about a factor of 2 different that of an adjacent band. The overall impulse responses are of interest because they can simplify the computation of the subband signals from a real-time stream by collapsing each cascade of filters into a single filter that can be used with the overlap-add method of convolution.

In other embodiments, rather than an iterated filterbank, a polyphase filter bank can be employed for wavelet decomposition of the EEG waveforms, as described in more detail below.

In many embodiments, the subband signals {d₄, d₅, d₆, d₇} are not directly used as the entries of a feature vector as such a representation of an input EEG sampled waveform can be too sensitive to both noise and slight variations in electrographic morphology commonly observed in the instances of a patient's seizures. Rather, four feature values that more generally summarize the information about the waveform components within the four subband signals are computed and employed as entries of a four-dimensional feature vector. For example, these values can be computed as functions of energies (signal strength) in the derived subbands. For example, the feature values can correspond to the absolute, rather than normalized, log-energies in each of the subband signals {d₄, d₅, d₆, d₇}. These quantities are particularly useful as quantitative measures for characterizing a waveform as they are sensitive to the amplitude of waveform components within each subband signal—an important discriminating factor that can be efficiently computed. Moreover, the nonlinear log operator used in computing these quantities amplifies small differences separating feature vectors of the seizure and non-seizure classes. An explicit representation of a feature vector X produced by the feature extraction stage in this embodiment can be represented as follows:

$$X = [ \begin{matrix}log ( {\sum }_{n} \vert {d}_{4} ( n ) \vert ) \\ log ( {\sum }_{n} \vert {d}_{5} ( n ) \vert ) \\ log ( {\sum }_{n} \vert {d}_{6} ( n ) \vert ) \\ log ( {\sum }_{n} \vert {d}_{7} ( n ) \vert )\end{matrix} ] \qquad Equation ( 1 )$$

wherein n refers to discrete data points in digitized representations of each subband.

In summary, the feature extraction stage can begin with a wavelet decomposition of an EEG waveform to produce subband signals that capture components contributing to the waveform morphology at specific time-scales or frequencies. Next the energy in each of these subband signals can be computed to form a feature value (also referred to herein as a statistic) that summarizes their activity while still being robust to noise and commonplace variations in the electrographic morphology of a patient's seizure onset.

### Classification

In the classification stage, feature vectors are assigned to either the seizure or non-seizure class by way of a classifier. The classifier reliably makes this binary assignment even though the feature vectors can represent more than two classes of activity. Specifically, as noted above, the non-seizure class can represent normal as well as artifact-contaminated EEG observed in different states of consciousness while the seizure class can represent EEG activity observed during seizure onset. In the embodiments describe herein, a probabilistic or a non-probabilistic classifier can be employed to determine the class membership of the observed feature vectors under both the SIP and SDP architectures. Descriptions of a probabilistic classifier, referred to as a maximum-likelihood classifier, and a non-probabilistic classifier, referred to as a support-vector machine, follow.

A maximum-likelihood classifier determines the class membership of a feature vector X by first computing the likelihood that the observation belongs to the seizure or non-seizure class, and then assigning the observation to the class with the greater likelihood. This classification criterion can be modified so that the observation is assigned to the class with a likelihood exceeding that of the other class by a specific factor, such as factory shown in the conditional relation below. The conditional probability density p(X|seizure) is the likelihood that the observed feature vector X belongs to the seizure class while the conditional probability density p(X|non-seizure) is the likelihood that it belongs to the non-seizure class. The determination of wherein X belongs to the seizure class can be based on the following criterion:

if

$$\frac{p ( X \vert seizure )}{p ( X \vert non \text{-} seizure )} \geq γ ,$$

the X belongs to seizure class.

The multi-dimensional likelihood functions p(X|seizure) and p(X|non-seizure) are a priori unknown, so their values for any observed feature vector X can be estimated by the classifier using the associated class's training examples and the nonparametric method of product-kernel density estimation. In essence, this density estimation technique equates the likelihood of a feature vector X to a sum of kernel functions K(z) that are stretched and shifted according to the spatial distribution of training samples X as shown in the following Equation (2):

$$p ( X ) = \frac{1}{n * {h}_{1} * … * {h}_{d}} {\sum }_{i = 1}^{n} {\prod }_{j = 1}^{d} K ( \frac{{X}_{j} - {\tilde{X}}_{ij}}{{h}_{j}} ) , \\ where \\ K ( z ) = \frac{1}{\sqrt{2 π}} exp ( - \frac{{z}^{2}}{2} ) \qquad Equation ( 2 )$$

This probability estimation is graphically illustrated for the one-dimensional case in FIG. 27. The figure shows instances of a Gaussian kernel centered over samples drawn from a one-dimensional random variable with unknown distribution, as well as the resulting bimodal density estimate that results from summing over the kernels. The bimodal density estimate explains well the clustering of the samples. The advantage of a nonparametric density estimate is that it makes no assumptions about the form of the likelihood functions in terms of the number or volume of modes, rather it extracts them from the training samples.

In the SIP architecture, a value for the threshold γ can be automatically chosen by each classifier to limit its individual probability of false-positive classification to a specified tolerance level α. More specifically, each classifier can search for a γ that satisfies the following Equation (3) using nonparametric estimates of the likelihood functions:

$$Z = \{ X \vert \frac{p ( X \vert seizure )}{p ( X \vert non \text{-} seizure )} \geq γ \} ; \\ {P}_{FP} = {\int }_{Z} p ( X \vert non \text{-} seizure ) d X \leq α \qquad Equation ( 3 )$$

The above Equation (3) states that a value of γ defines a decision region Z where the classifier will assign all observed feature vectors X to the seizure class. The decision region Z can be a single region or the union of several disjoint regions. Furthermore, the probability of a false-positive classification given a value of γ is given by an integral over the region Z of the likelihood of X belonging to the non-seizure class. The value of γ is preferably chosen by the classifier so that this integral results in a probability of false-positive classification that is less than α. Once an appropriate γ is determined by each classifier, their individual probabilities of true-positive classification is given by the following Equation (4). These probabilities can utilized in a manner described below for spatially localizing a patient's seizure onset.

$${P}_{TP} = {\int }_{Z} p ( X \vert seizure ) d X . \qquad Equation ( 4 )$$

In the SDP architecture, the high dimensional feature vectors can make the approximation of the integrals in Equation (3) difficult. Consequently, in many embodiments utilizing the SDP architecture, the value of γ is not set according to a specified tolerance on false-positive classification. Rather, it is determined empirically and fixed across patients as discussed in more detail below.

To further elucidate the maximum-likelihood classification methodology, an example of a decision region computed by a maximum-likelihood classifier using a sample training set is illustrated in a two-dimensional space. A two-dimensional feature vector X′ within this space can be synthesized by projecting a four-dimensional feature vector X used by the SIP architecture onto the directions of greatest variance φ₁ and φ₂ computed by utilizing principle components analysis, as shown in Equation (5) below:

$${X}^{′} = [ \begin{matrix}{X}_{1}^{′} \\ {X}_{2}^{′}\end{matrix} ] = [ \begin{matrix}{φ}_{1} \cdot X \\ {φ}_{2} \cdot X\end{matrix} ] . \qquad Equation ( 5 )$$

The patient-specific training feature vectors used by the maximum-likelihood classifier to determine an exemplary decision region are illustrated in FIG. 28. These feature vectors were computed by passing seizure and non-seizure epochs from one derivation through the feature extraction stage, and then transforming the resulting four-dimensional feature vectors X into lower-dimensional feature vectors X. Note the number of non-seizure training examples is greater than seizure onset training examples. This is typical of many training sets since there are generally more non-seizure EEG waveforms to sample from a patient than seizure onset EEG waveforms.

The first step in determining a decision region Z involves using the training feature vectors and kernel density estimation to construct estimates of the seizure and non-seizure likelihoods, as shown in FIG. 29A. These estimates are then used in the above Equation (3) to compute the decision region Z, illustrated in FIG. 29B, that limits the probability of a false-positive classification to a maximum value of α. Increasing the value of α will result in a decision region with a greater radius, and consequently the correct classification of more seizure examples at the expense of the incorrect classification of more non-seizure examples.

In many embodiments, particularly those that employ the SDP architecture, a non-probabilistic methodology is employed for classification of a feature vector. For example, in such embodiments, a support vector machine can be utilized for determining the class membership of a feature vector X based on which side of an optimal hyperplane the feature vector lies. In the case of linearly separable classes, this optimal hyperplane can be the one that is maximally distant from support-vectors. These are the training examples from both classes corresponding to boundary cases, and consequently the ones carrying all relevant information about the classification problem. If the classes are not linearly separable, the optimal hyperplane can be determined in a higher-dimensional feature space where they are linearly separable—this translates to computing a nonlinear decision boundary in the original space.

A kernel is a function that allows support-vector machines to define the optimal hyperplane in a kernel-specific, higher-dimensional space without the explicit construction of high-dimensional feature vectors. In some embodiments of seizure-onset detection methods of the invention, the Radial-Basis Kernel expressed in Equation (6) below can be chosen since determination of an optimal hyperplane in its associated high-dimensional feature space can yield nonlinear decision boundaries that may be discontinuous when necessary. In other words, the decision region of a Radial-Basis Kernel need not be a single region, rather it can be the union of several disjoint regions.

$$Radial \text{-} Basis Kernel \text{:} K ( {X}_{i} , {X}_{j} ) = exp ( - \frac{{‖ {X}_{i} - {X}_{j} ‖}^{2}}{2 {σ}^{2}} ) , \\ where σ \geq 0 \qquad Equation ( 6 )$$

The ability of a support vector machine to discriminate between two classes can be influenced by their separability, the parameters of the chosen kernel, and the class-specific penalty for determining a decision boundary that misclassifies a percentage of training examples. In the case of the Radial-Basis Kernel, decreasing its parameter σ translates into increasingly sophisticated boundaries that correctly classify a higher percentage of training examples. Similarly, increasing the penalty for misclassifying the training examples of a given class encourages the determination of a decision boundary that correctly classifies those examples—the penalties can he specified independently for each class through the two entries of a vector parameter C². Extreme choices for both of these variables can increase the risk of overfitting. In other words, it can lead to creation of a classifier that correctly identifies a high percentage of the training set, but performs poorly on an unseen test set. The risk of overfitting can be gauged by the percentage of training examples considered as support vectors—the greater the percentage the higher the risk of overfitting.

As described in more detail below, in the SIP architecture, the probabilities of true and false-positive classification of each classifier can be employed to localize a patient's seizure onset. In the case of support vector machines, these probabilities can be approximated by employing the following relations:

$${P}_{TP} \approx \frac{{N}_{correct seizure}}{{N}_{total seizure}} ; {P}_{FP} \approx \frac{{N}_{incorrect normal}}{{N}_{total normal}} \qquad Equation ( 7 )$$

To further elucidate the formation of a decision region by employing a support vector machine and only for illustrative purposes, computation of an exemplary decision region in a two-dimensional space by a support vector classifier operating on a training set is now described. Similar to the previous classification example, two-dimensional feature vectors X′ within this space can be synthesized by projecting a four-dimensional feature vector X used by the SIP architecture onto the directions of greatest variance φ₁ and φ₂ computed by utilizing principle components analysis, as shown in Equation (8) below:

$${X}^{′} = [ \begin{matrix}{X}_{1}^{′} \\ {X}_{2}^{′}\end{matrix} ] = [ \begin{matrix}{φ}_{1} \cdot X \\ {φ}_{2} \cdot X\end{matrix} ] \qquad Equation ( 8 )$$

The patient-specific training feature vectors used by the support vector machine to determine a decision region, which are illustrated in FIG. 30 are equivalent to those used in the classification example of the maximum-likelihood classifier. The feature vectors were computed by passing seizure and non-seizure epochs from one derivation through the feature extraction stage, and then transforming the resulting four-dimensional feature vectors X into lower-dimensional feature vectors X′.

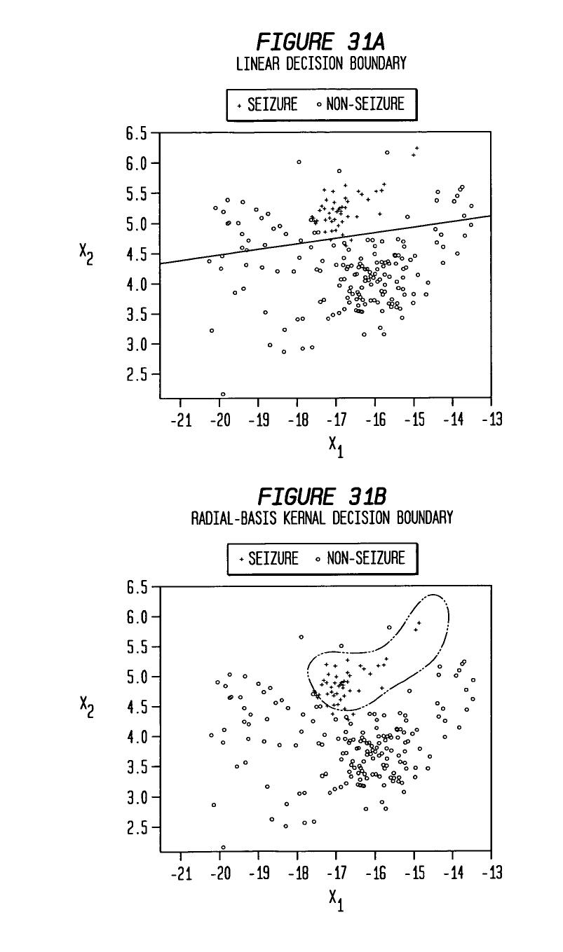

The support-vector machine classifier uses the training feature vectors to compute the coefficients parameterizing the optimal hyperplane in either the original or kernel-induced feature space. Computing the hyperplane in the original feature space leads to the linear decision boundary shown in FIG. 31A while computing the hyperplane in the feature space induced by a radial basis kernel with parameter σ=1 is shown in the FIG. 31B. The nonlinear decision boundary computed by the support vector machine is very different from that determined by the maximum-likelihood classifier, which is not unexpected given the vastly different theoretical foundation of each classification scheme.

### Spatial and Temporal Constraints

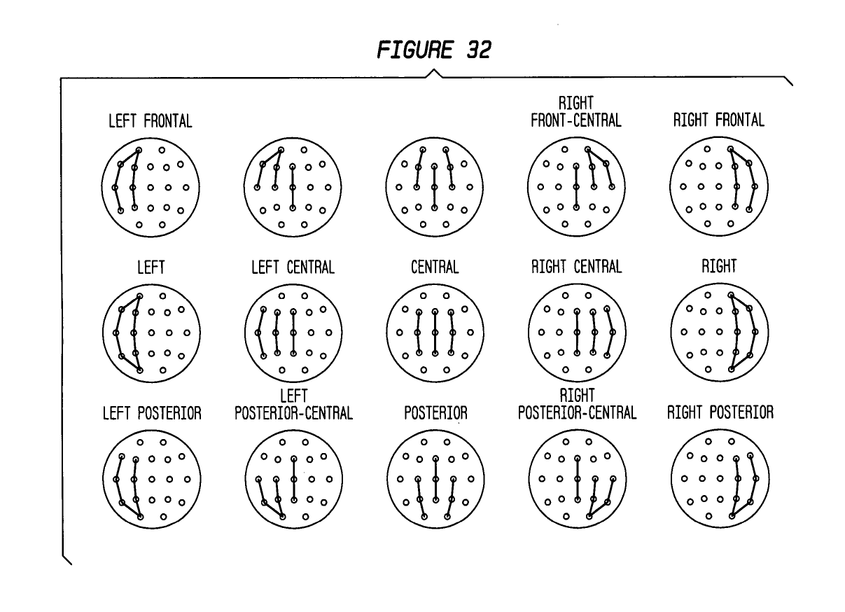

In the SIP architecture, the assigned class memberships of the feature vectors are examined in the context of temporal and patient-specific localization constraints in order to make a final decision regarding seizure onset (in the embodiments discussed herein twenty-one feature vectors corresponding to twenty-one waveform channels are examined, in other embodiments the number of feature vectors can be different). Specifically, a seizure-onset detector according to an embodiment of the invention utilizing the SIP architecture can be programmed to declare seizure onset only after K derivations are assigned to the seizure class for a duration of T seconds. By way of example, the K derivations can all belong to one of the groups illustrated in FIG. 32. The choice of one or more derivations for a given patient can depend on the nature of that patient's seizures and can be determined automatically by the detector, as discussed below. The groups in FIG. 32 can provide coverage of possible centers of focal seizure activity; moreover, in the case of generalized seizures any one of these groups can be used for the purpose of detection since all derivations will be active at the seizure's onset.

For a given patient, the detector can choose the group exhibiting the highest level of discrimination between non-seizure and seizure activity on its constituent derivations. This can be accomplished, for example, by first assigning each derivation a weight based on the ability of its classifier to differentiate between seizure and non-seizure activity, and then selecting the group with the maximal total weight. A weight aᵢ assigned to derivation i can be computed by employing its classifier's probability of true and false-positive classification as expressed in Equation (9) below while an optimal group Gⱼ can be the one with the greatest total weight wⱼ shown in Equation (10) below.

aᵢ=P_{TP,i}−P_{FP,i} i=1, . . . , 21 Equation (9),

where i corresponds to waveform channels (in this embodiment 21 channels are observed).

$${w}_{j} = {\sum }_{k ∈ {G}_{j}} {a}_{k} j = 1 , … , 15 \qquad Equation ( 10 )$$

### Training

In many embodiments of the invention, during training, the classifiers use a diverse set of examples from the seizure and non-seizure classes to determine decision boundaries. By way of example, in embodiments in which 21 derivations are employed, the training examples can be patient-specific, non-overlapping sets Sᵢ=1, . . . , 21, each containing selected epochs (e.g., two-second epochs) of labeled activity from a single EEG derivation. The epochs that correspond to seizure-related activity are labeled as examples of the seizure class, while those corresponding to both normal and artifact-contaminated activity from different states of consciousness are labeled as examples of the non-seizure class. It should be understood that training sets can be constructed in a similar manner in embodiments that utilize different number of derivations or employ referential recordings.

The training procedure can begin by converting the labeled sets Sᵢ into a collection of feature vectors {X} by passing their epochs through the feature extraction stage. The feature vectors are used by the classifiers for the purpose of estimating quantities necessary for defining a decision boundary. In the case of maximum-likelihood classifiers, these quantities correspond to the conditional densities pᵢ(X|seizure) and pᵢ(X|non-seizure) while for support-vector machines the quantities are the coefficients of the hyperplane in the kernel-induced feature space.

To further illustrate the salient features of methods and systems of the invention for detecting seizure onset, several case studies are discussed below. It should be understood that these examples are provided only for illustrative purposes and are not necessarily intended to indicate an optimal performance of a seizure-onset detector constructed based on the teachings of the invention.

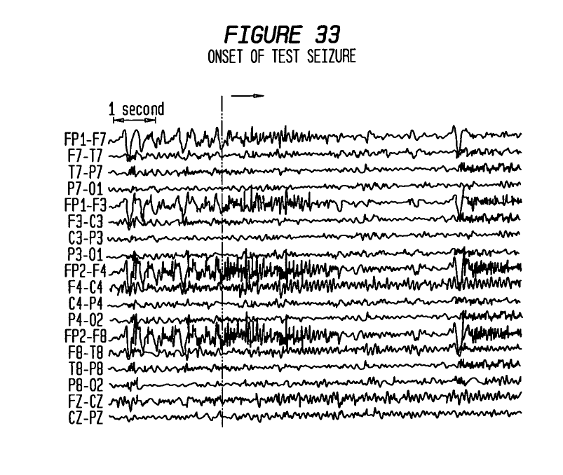

Case 1: As the first example, consider detecting the electrographic onset of the seizure illustrated in FIG. 33 by employing a detector according to the teaching of the invention having the SIP architecture. This seizure's onset is characterized by a paroxysmal, 10 Hz burst of sharp and monomorphic waves localized to the central derivations {Fz-Cz; Cz-Pz}, the right fronto-central derivations (FP₂-F₈;F₄-C₄), and the right frontal derivations {FP₂-F₈;F₈-T₈;T₈-P₈}. With the exception of {FP₁−F₇; FP₁-F₃}, the derivations on the left side of the head, which are odd-numbered, show no appreciable change in behavior after the onset. These characteristics imply that the seizure originates from a region towards the front and right-side of the head.

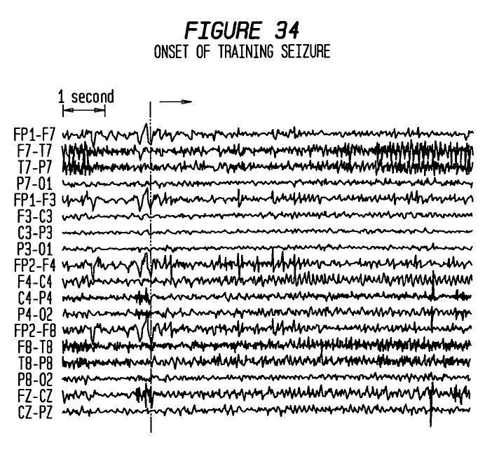

The first step in the detection process is to train the detector not only on 2-4 previous occurrences of seizure onsets similar to that illustrated in FIG. 33, but also on the non-seizure EEG separating these occurrences. FIG. 34 shows one of the training seizures presented to the detector, which is very similar to the one to be detected except for less prominent activity on the frontal derivations {FP₁-F₇; FP₁-F₃; FP₂-F4; FP₂-F₈}. This difference illustrates the variability between the instances of a seizure, and explains why the detector typically requires more than one training seizure in order to discover the derivations that are consistently active following the electrographic onset. The training seizure is not used as it is shown in the figure. Rather, it is segmented into two-second epochs that are grouped into the training sets Sᵢ=1, . . . , 21 according to their source derivation.

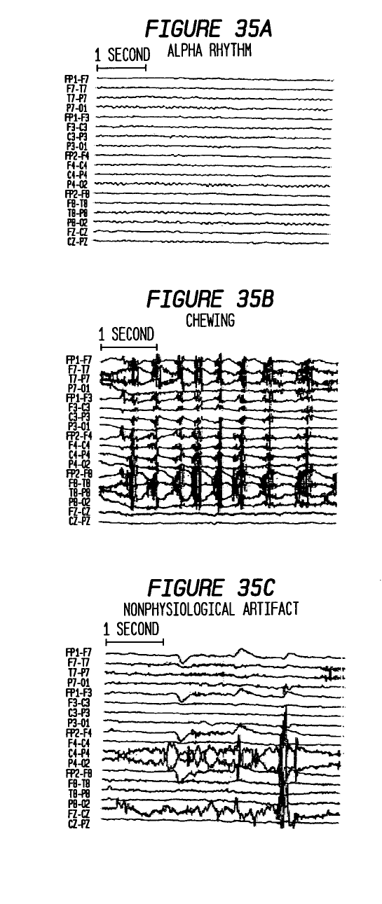

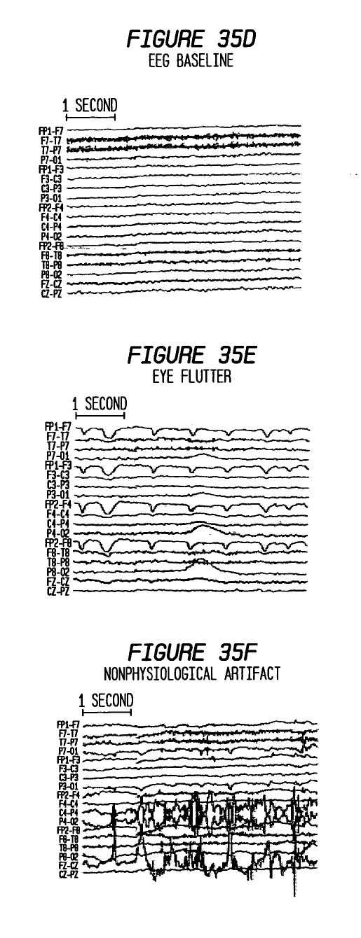

As shown in FIGS. 35A-35F, the non-seizure EEG waveforms included as part of the detector's training consist of the baseline EEG; rhythms from different states of consciousness such as the normal alpha rhythm, physiological artifacts such as those caused by eye flutter or chewing, and nonphysiological artifacts such as those introduced by movement of EEG electrodes. Since nonphysiological artifacts are not necessarily limited to the derivations on which they are observed, they are artificially introduced into the training set Sᵢ of each classifier. In all other cases, the training sets Sᵢ only contain epochs of EEG from a single derivation.

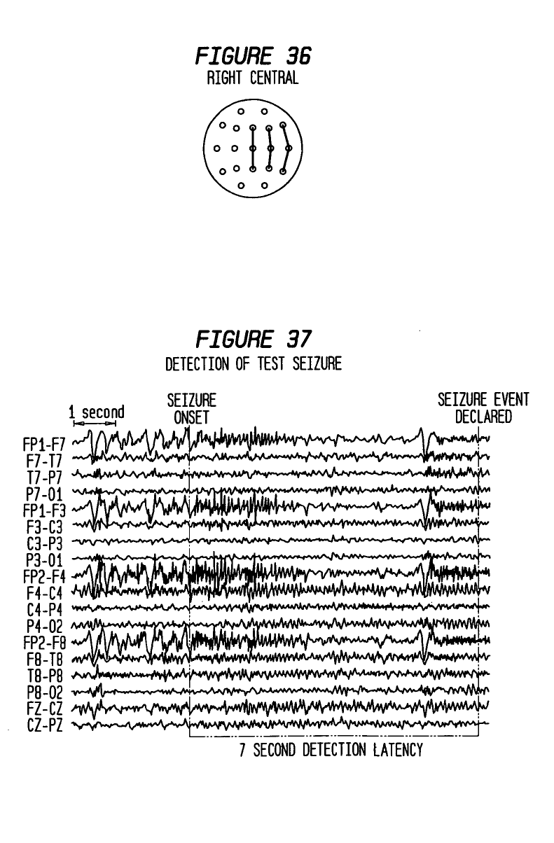

After the epochs within the training sets Sᵢ are converted to sets of feature vectors, the detector determines the decision boundary associated with each classifier. For instance, the maximum-likelihood and support vector machine decision boundaries for the derivation {F₄-C₄} are shown in FIGS. 29A and 31. The detector uses the decision boundaries to compute the probabilities of true and false-positive classifications P_{TP,i} and P_{FP,i} so as to localize the seizure's onset to one of the groups in FIG. 32. In this example, the detector selects the right-central derivations shown in FIG. 36. All the selected derivations exhibit a change in their waveforms following seizure onset with the possible exception of {C₄-P₄}; this result illustrates that a consequence of selecting derivations as a group is the possible inclusion of irrelevant derivations, and also explains why the detector performs relatively poorly when declaration of a seizure event is conditioned on observing seizure activity on K=6 rather than K<6 derivations. Note that specifying a minimum number of derivations for declaring a seizure event is not required by the SDP architecture since spatial localization constraints are encapsulated within its feature vectors, rather than explicitly imposed as in the SIP architecture.

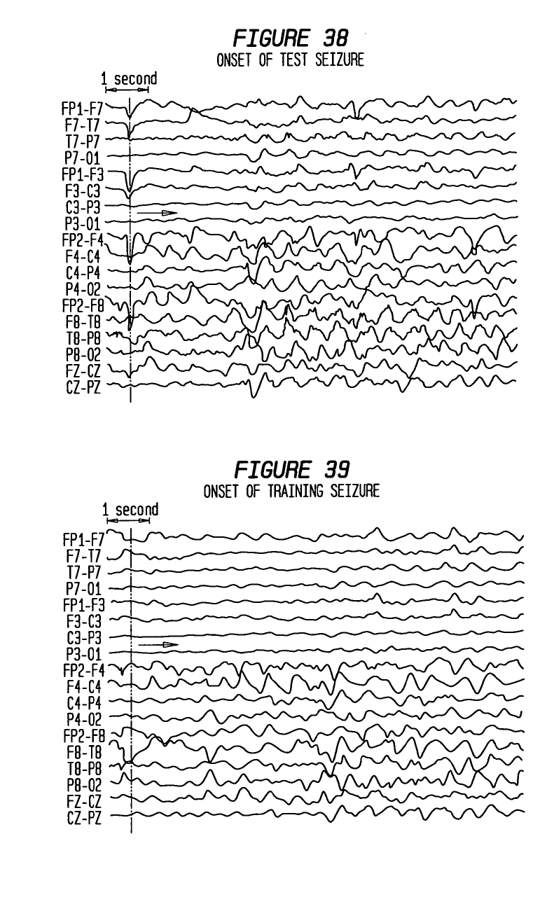

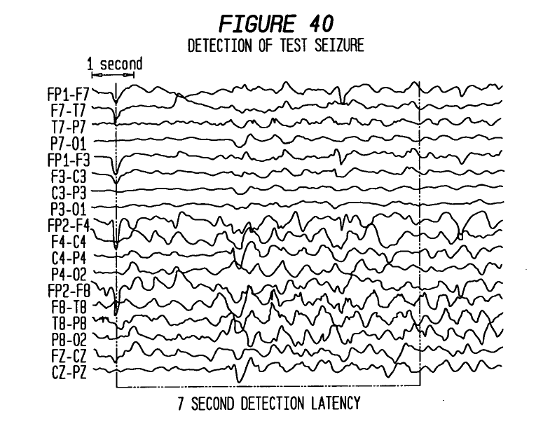

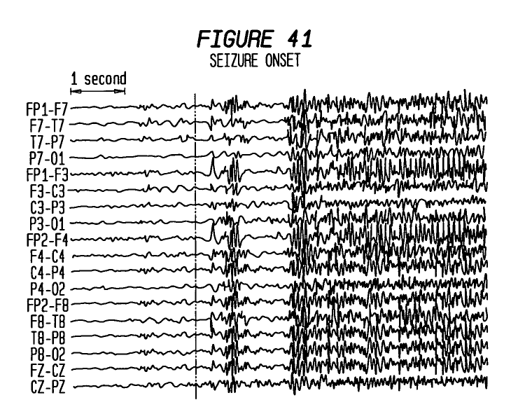

When the trained detector was used to detect the test seizure using K=4 derivations and T=6 seconds, a seizure event was declared seven seconds following the electrographic onset as shown in FIG. 37. The derivations responsible for triggering the detection included {F₄-C₄; F₈-T₈; T₈-P₈; F_{z}-C_{Z}; C_{Z}-P_{Z}}. On the other hand, the abnormal activity on the frontal derivations {FP₁-F₃; FP₁-F₇; FP₂-F₄; FP₂-F₈} was not used for the purpose of detection because these derivations are not members of the selected group. Even if the frontal derivations were members of the selected group they would not have triggered a detection since their seizure activity does not persist for the required T=6 seconds.

Case 2: This case study highlights the importance of both localization and morphology to seizure detection, and the possibility of sharing certain types of non-seizure activity across the training sets of patients. Consider detecting the electrographic onset of the seizure illustrated in FIG. 38 again using a detector according to the teachings of the invention having the SIP architecture. This seizure's onset is characterized by a paroxysmal 2 Hz burst of monomorphic waves localized to the central derivations {F_{Z}-C_{Z}; C_{Z}-P_{Z}}, and all derivations on the right-side of the head {FP₂-F₄; F₄-C₄; C₄-P₄; P₄-O₂; FP₂-F₈; F₈-T₈; T₈-P₈; P₈-O₂}. The baseline EEG can be observed on derivations from the left-side of the head, which are odd-numbered, since they exhibit no change after the onset. This electrographic evidence indicates that the seizure originates from the right-side of the head.

To detect the test seizure shown electrgraphically in FIG. 38, the detector needs to be trained on previous instances of the seizure as well as on non-seizure EEG separating these instances as was done in the above Case 1. It is interesting to note that the baseline EEG included as part of the non-seizure training must be specific to the case; in contrast, physiological and nonphysiological artifacts as well as hallmark activity from different states of consciousness can be shared across cases within similar age groups. This is supported by the fact that an electroencephalographer can identify these activities solely based on morphology, localization, and reactivity; reference to the baseline EEG associated with the case is not necessary. In contrast, an electroencephalographer cannot be certain whether an epoch of activity includes seizure onset without reference to the baseline EEG, which argues for the necessity of baseline and seizure EEG to be case-specific. Hence, in some embodiments, a diverse library of case-independent physiological and nonphysiological activity can be compiled and saved, and then used to supplement the baseline and seizure EEG that are specific to the case under consideration. By way of example, FIG. 39 shows one of the training seizures presented to the detector.

Following training and completion of the localization procedure (discussed above), the detector selected the right-central derivations shown in FIG. 36. While the selected group of derivations matches that of Case 1, the detector from Case 2 fails to detect the test and training seizures from Case 1 because of the very different waveform morphologies. This demonstrates the role of both morphology and localization to seizure onset detection.

When the trained detector of this case was used to detect the test seizure in FIG. 38 using K=4 derivations and T=6 seconds, a seizure event was detected seven seconds following the electrographic onset as shown in FIG. 40. The six derivations responsible for detection included {F₁-C₄; C₄-P₄; F₈-T₈; T₈-P₈; FZ-CZ; Cz-P_{Z}}.

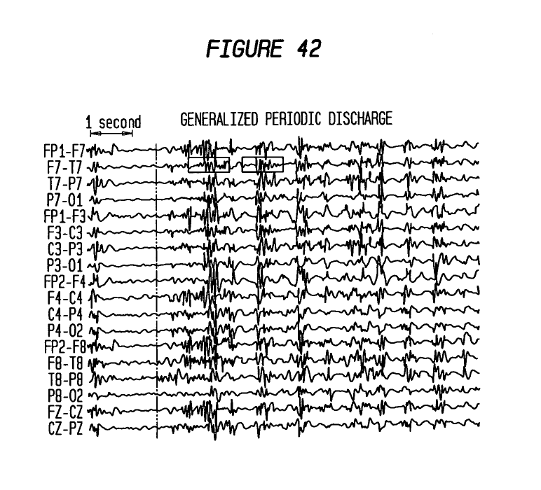

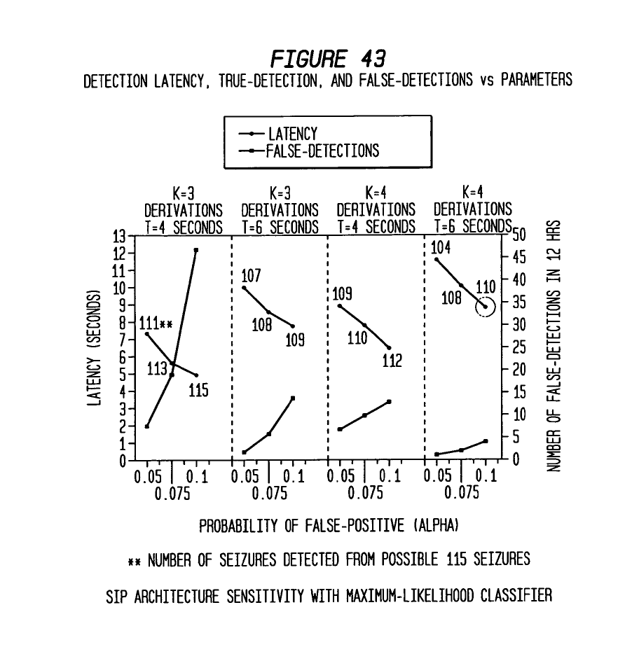

Case 3: This case study relates to detection of EEG abnormal discharges that can occur between seizure events. Such events may have similar morphology and localization as actual seizures. Consider a detector with the SIP architecture that is trained on several electrographic seizure onsets similar to that shown in FIG. 41. Since the onsets are generalized, the detector can select any of the group of derivations illustrated in FIG. 32 for subsequent detections. When the trained detector was presented with non-seizure EEG between seizure occurrences, a false seizure event was declared upon analyzing the generalized, periodic discharge of sharp-wave groups boxed in FIG. 42 following the dotted line. The sharp wave groups in FIG. 42 can be visually distinguished from those in FIG. 41 by their temporal spacing. To the detector utilized for this study, both activities appear similar since the spacing between any two groups of shape waves does not exceed two-seconds, the duration with which EEG is analyzed. In other embodiments, longer epochs can be utilized to sample the waveform to avoid detecting such inter-ictal discharges. However, the detection of such inter-ictal discharges can be useful in some applications, such as, vagus nerve stimulation discussed below.

The performances of exemplary seizure onset detectors formed in accordance with the teachings of the invention with SIP and SDP architectures were further assessed by employing the detectors to identify seizure onsets in thirty-six de-identified test subjects. This test data is presented only for illustrative purposes and is not intended to necessarily present optimal performance characteristics of seizure onset detectors of the invention.

A detector's performance was gauged by employing the following metrics computed for each subject: Detection Latency (an average time elapsed between the electrographic onset of a seizure and the declaration of a seizure event); True-Detections (the number of test seizures declared as seizure events); and False-Detections (the number of false-positives declared during analysis of non-seizure EEG).

In general, improving a detector's performance as measured by one or two of these metrics may result in a lower performance as measured by the other metric(s). For example, while decreasing the detection parameter T will result in shorter detection latencies and a possible increase in the number of true-detections, it may also result in an increase in the number of false detections. Such increase in false-detections can result, for example, from short-duration, seizure-like discharges commonly observed in the EEG waveforms during periods that separate seizure events. The number of true-detections will increase or remain unchanged depending on whether or not the original value of T resulted in misses of very short-duration seizure events.

For each test subject, four or five bipolar EEG recordings sampled (digitized) at 256 Hz, and each containing a seizure event with an onset labeled by an experienced electroencephalographer were available. The recordings lasted approximately 20 minutes for twenty-four of the subjects; 40 minutes for six of the subjects; 150 minutes for four of the subjects; and 12 hours for remaining two subjects. For each subject, a leave-one-out cross-validation testing scheme was followed. In particular, the detector was trained on a training set that included the seizure and non-seizure epochs from all but one of the subject's recordings, and was then used on the excluded recording. This was repeated until each recording had been excluded once. The training set was also supplemented with a library of epochs that included generic artifacts and hallmark activity from various states of consciousness, for example, sleep spindles from the second stage of sleep. This can compensate for potential under representation of activity types in the training recordings. As a practical matter, it implies that training records can he assembled quickly and without a great deal of concern over whether or not they are truly representative.

In short, a subject with recordings {A B C D} would require the following four testing trials:

Trial 1: Train on {A B C EEG Library} and test on recording D;

Trial 2: Train on {A B D EEG Library} and test on recording C;

Trial 3: Train on {A C D EEC Library} and test on recording B;

Trial 4: Train on {B C D EEG Library} and test on recording A.

The performance metrics reported include the average detection latency; the number of test seizures detected and the total number, as opposed to the hourly rate, of false-detections. For a given subject, the reported detection latency is the average of latencies measured in each testing trial, while the reported number of true and false-detections is the sum of seizures and false-positives declared in all the testing trials. The average detection latency corresponds closely to the desired “expected latency” metric. Also, once the number of test seizures detected is normalized by the total number of available test seizures, it will closely approximate the metric “percentage of seizures likely to be detected.”

Reporting the total number of false-detections equally weighs false-detections declared in the short-length recordings of one patient with those in the long-length recordings of another. In other words, a false-detection caused by a movement artifact in a twenty-minute recording is not treated differently from the same false-detection in a thirty-minute recording.

In the SIP architecture, the detector's performance can be influenced by the choice of several parameters that directly control when seizure onset is declared. These parameters are: the required duration time T of an abnormality; the minimum number of derivations K exhibiting the abnormality; the allowable probability of false-positive classification α for maximum-likelihood classifiers, and the radial-basis kernel parameter σ and vector parameter C for support vector machines. The parameters α, σ, and C may be freely set for each classifier in the SIP architecture, but one value for each parameter was used to reduce the detector's degrees of freedom across all of them.

FIG. 43 illustrates the change in performance of a detector that combines the SIP architecture with maximum-likelihood classifiers due to different choices of the parameters T, K, and σ. This figure shows that for a given choice of T and K, increasing the probability of false-positives resulted in a decrease in the average detection latency, and an increase in both the true and false-detections measured for twenty-eight of the thirty-six subjects.

The optimal choice of parameter settings can depend on the detector's application. For instance, if the detector is used to activate harmless stimulation of brain regions upon detecting a seizure, then false-detections are not costly but minimizing latency can be crucial. In such a case, the parameter settings T=4 seconds, K=3 derivations, and α=0.10 may be appropriate. In our application, both latency and false-detections were minimized by employing the parameter settings T=6 seconds, K=4 derivations, and α=0.10, as shown by the circled data point in FIG. 43.

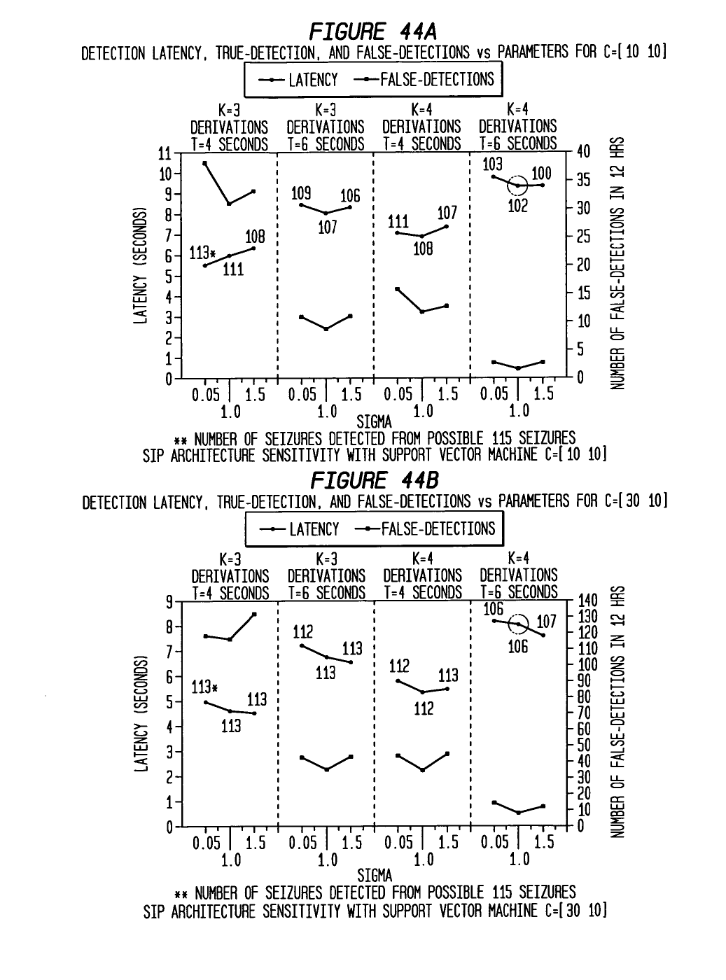

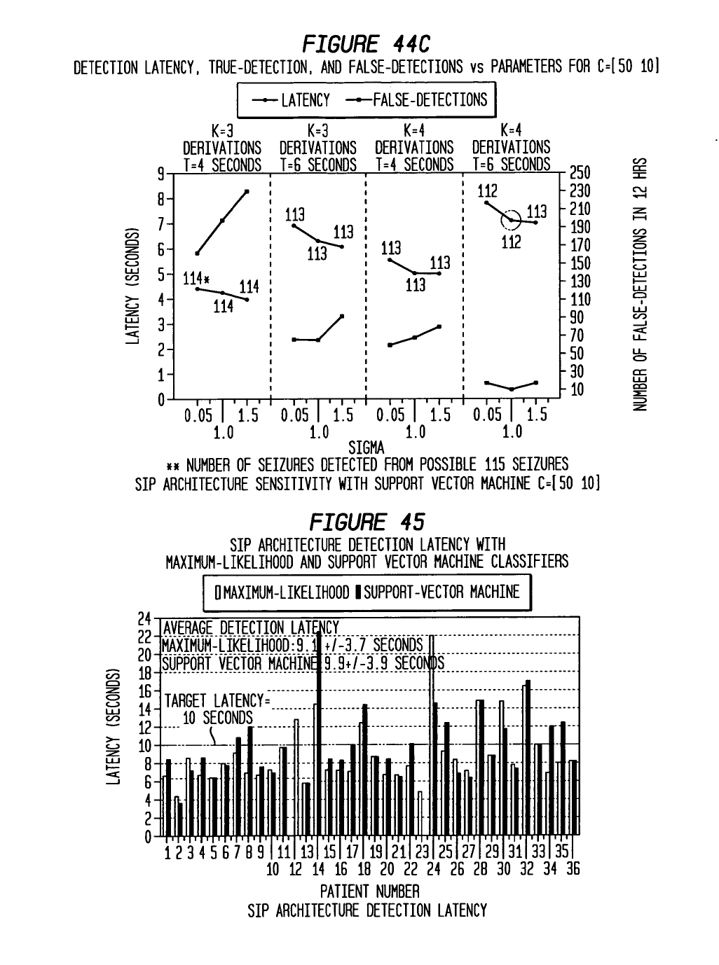

The sensitivity of a detector that combines the SIP architecture with support vector machines to changes in T, K, σ, and C is illustrated in FIGS. 44A-44C. For a given choice of the vector C, whose first and second entries corresponds to the cost of misclassifying seizure and non-seizure training examples respectively, the values of T and K are responsible for changes in the average detection latency and total number of true and false-detections. In contrast, the performance metrics remain almost constant for changes in σ. The values of σ were chosen so that decision boundaries required between 10%-40% of the training data to be support vectors, a percentage that limits the prospect of overfitting. The parameter settings C=[10 10], T=6 seconds, K=4 derivations, and σ=1 minimize both latency and false-detections as measured for twenty-eight of the thirty-six subjects (this data point is circled in FIG. 44A).

Although the detector can exhibit a lower detection latency and a higher true-detection rate with C=[30 10] and C=[50 10], as shown by the boxes in FIGS. 44B and 44C, the circled parameter settings that include C=[10 10] exhibit a lower number of false-detections.

For the parameter settings T=6 seconds, K=4 derivations, α=0.10, σ=1, and C=[10 10], FIG. 45 compares the average detection latency of an exemplary detector that combines the SIP architecture with maximum-likelihood classifiers with that of a similar detector that employs support vector classifiers.

The detection latencies for both configurations are similar, indicating that these exemplary detectors are not highly sensitive to the classifier type. Furthermore, the detection latencies for most subjects are less than a target latency of ten seconds by more than one second. For subjects 12 and 23, a zero detection latency is shown since the support vector machine based detector failed to identify any seizure events. However, when the parameter C was changed from C=[10 10] to C=[30 10], the support vector machine managed to correctly classify seizure waveforms with a latency matching that of the maximum-likelihood classifier, but at the expense of two extra false-detections on subject 12. The same change in C also reduced the latency of the support vector machine based detector on subject 14 to the level shown for the maximum-likelihood based detector. Finally, the large latencies shown for subjects 14 and 24 are understood to be the result of gradual seizure onsets localizing to a number of derivations less than the required detection minimum of K=4 before spreading to include a greater number of derivations.

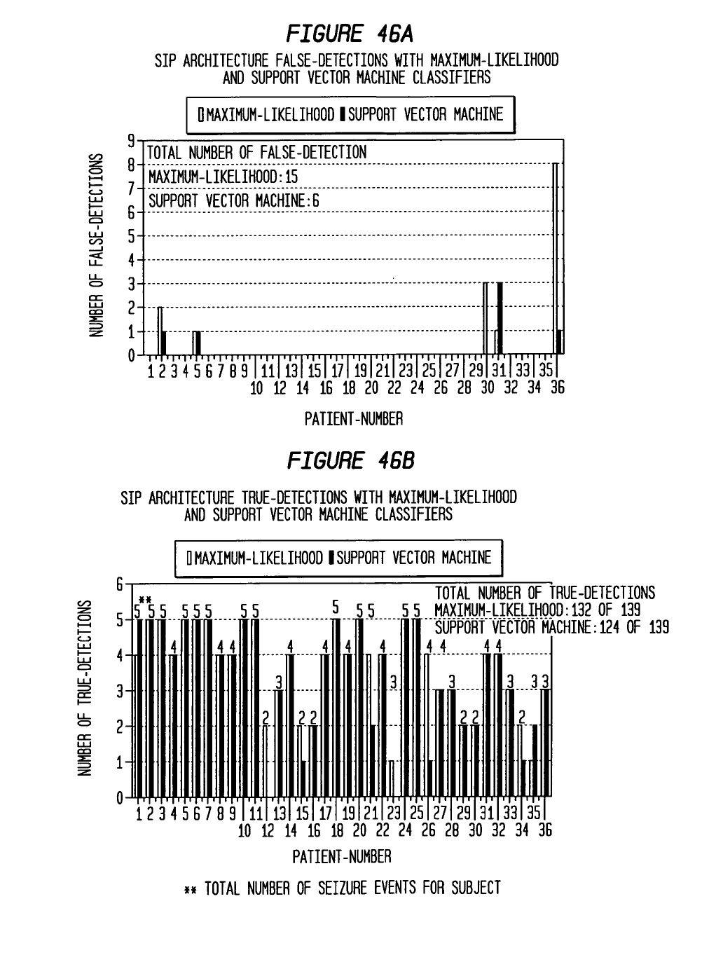

FIG. 46A shows the false-detections declared on each test subject for both detector configurations. With the exception of subject 30 whose false-detections were a result of non-physiological artifacts, all the false-detections declared by both detector types were caused by periodic discharges resembling the seizure onset activity of the particular subject. The maximum likelihood classifier based detector was especially sensitive to the periodic discharges of subject 36, this lead to eight false detections in twelve hours of processing.

FIG. 46B also shows the true-detections declared on each test subject for both detector configurations (the number over each bar denotes the number of test seizures for a given subject). The discrepancy in true-detections between detector types is caused by the conservative choice of C=[10 10], which leads the support vector machine based detector to miss more seizures from subjects 12, 21, and 23. When C=[30 10] was used, the support vector machine based detector identified the same number of seizures for these subjects as the maximum-likelihood detector, but at the expense of more false-detections on other subjects.

As discussed in detail above, in the SDP architecture, localization constraints are encapsulated within a composite feature vector. Thus, the detector's performance can be influenced by the required duration time T of an abnormality; the likelihood ratio threshold γ in the case of maximum-likelihood classifiers, and both the radial-basis kernel parameter σ and vector parameter C in the case of support vector machines.

The sensitivity in performance of a detector with the SDP architecture and maximum-likelihood classifiers due to different choices of the parameters T and γ is illustrated in FIG. 47. The figure shows that for a given choice of T, increasing the threshold γ can result in an increase in the average detection latency, and a decrease in both the true-detections and false-detections measured for twenty-eight of the thirty-six subjects. To optimize performance primarily in terms of latency and false-detections, the parameter settings T=6 seconds and γ=10² were chosen because they provide an appropriate tradeoff between these metrics, as shown by the circled data point in FIG. 47.

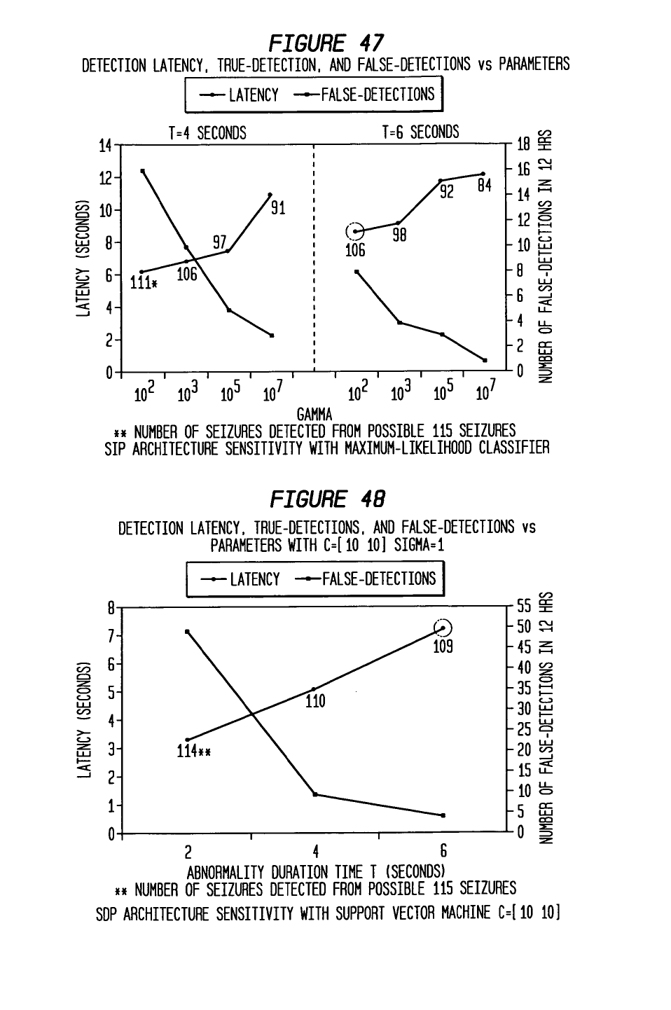

FIG. 48 illustrates the sensitivity of a detector that combines the SDP architecture with a support vector machine to different values of the parameter T (the settings σ=1 and C=[10 10] were fixed having observed their effects on performance through the SIP architecture). FIG. 48 shows that increasing the parameter T increases the average detection latency and decreases both the true and false-detections measured for twenty-eight of the thirty-six subjects. For this detector configuration the parameter settings T=6 seconds, C=[10 10], and σ=1 resulted in a tradeoff between detection latency and false-detections, as shown by the circled data point in FIG. 48.

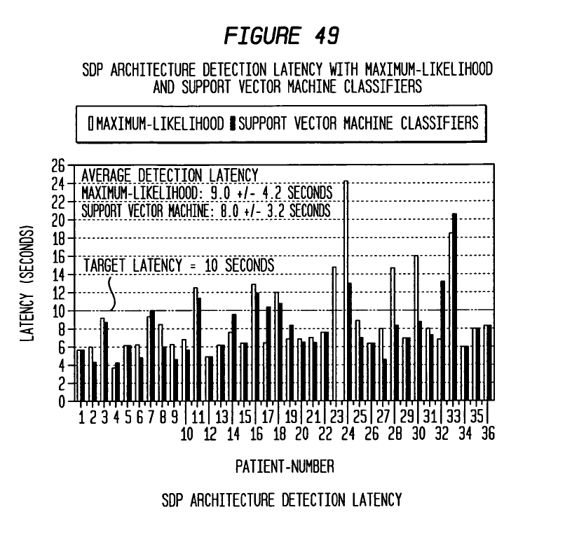

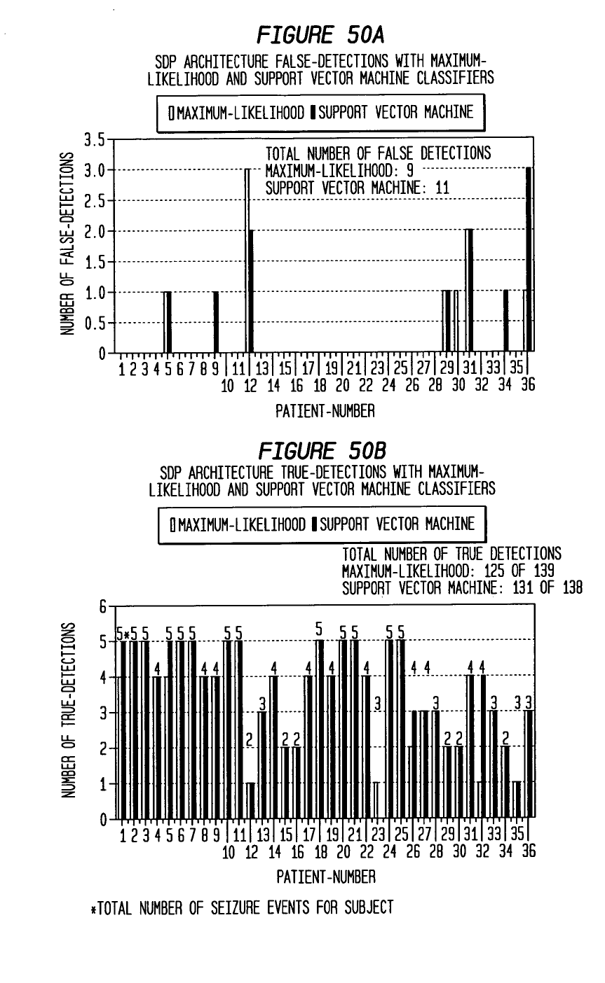

For the parameter settings T=6 seconds, γ=10², C=[10 10], and σ=1, FIG. 49 shows the average detection latency of a detector that combines the SDP architecture with a maximum-likelihood classifier or a support vector machine. The latencies of both detector configurations are similar. Furthermore, the detection latencies of most subjects are less than a target latency of ten seconds by more than two seconds. The conservative choice of C=[10 10] as well as gradual seizure onsets resulted in relatively poor performance in subjects 23 and 24, while an artifact masking seizure onset activity on a number of derivations resulted in a fairly poor performance on subject 33. Coincidentally, the artifact did not affect the performance of the SIP architecture since it was not present on the selected derivations. On the other hand, the SDP architecture did not exhibit a latency for subject 14 that is as large as that of the SIP architecture since there was no explicit setting in the SDP architecture for the minimum number of derivations required for a detection.

FIG. 50A shows the false-detections declared on each test subject for both detector configurations. With the exception of subjects 9, 29, and 30 whose false-detections are a result of non-physiological artifacts, all other false-detections are a result of periodic discharges that resemble the seizure onset of a particular subject. The support vector machine based detector was more sensitive to discharges of subject 36.

FIG. 50B shows the true-detections declared on each test subject for both detector configurations. The difference in true detections is primarily caused by the three seizure events from subject 32 that were missed by the maximum-likelihood based detector. Lowering the value of y would most likely allow for the detection of these seizures at the cost of more false-detections.

As was discussed, a fundamental difference between the SIP and SDP architectures is the manner of representing and enforcing spatial localization constraints. In the case of the SIP architecture, these constraints are imposed by employing explicit rules in the final element of the detector. This permits independent classification of activity on each derivation in a low dimensional feature space, and the skipping of derivations that are irrelevant to the detection of a seizure's onset. In contrast, the SDP architecture expresses spatial constraints through the interrelations of elements within a composite feature vector summarizing activity from all derivations. While this obviates the need to explicitly enforce localization constraints, it can hide from the user information regarding the derivations that are utilized for detection, and it can cause classification to take place in a higher dimensional space that may include features irrelevant to the detection of a given seizure's onset. A brief comparison of exemplary SDP an SIP architectures are now provided.

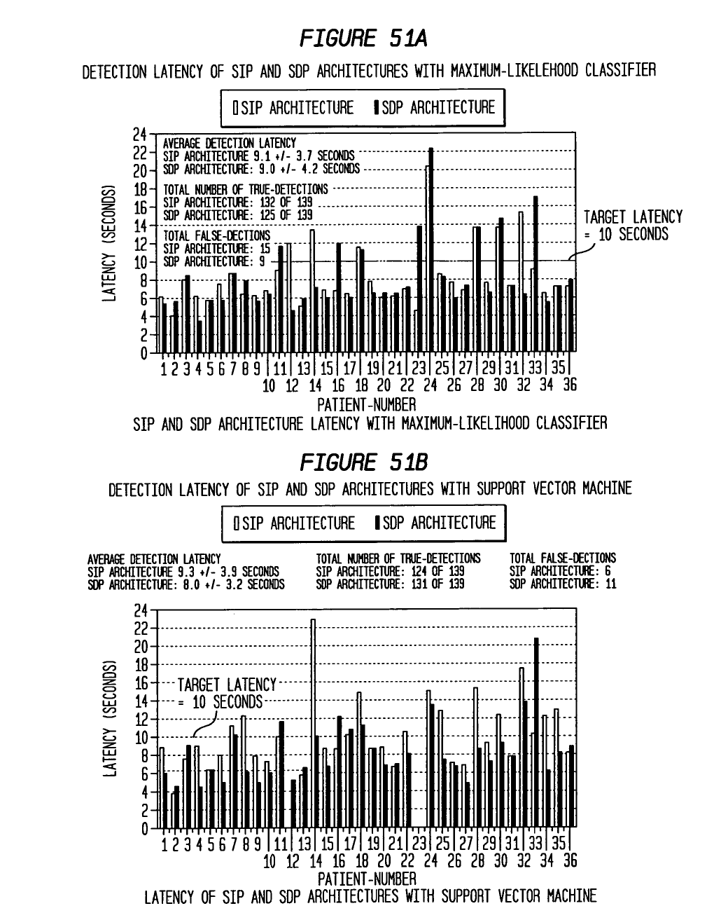

FIG. 51A compares the performance of the exemplary SIP and SDP architectures when combined with the maximum-likelihood classifier in the above study. The two architectures exhibit similar detection latencies across all subjects, but the SIP architecture exhibits a slightly higher number of true-detections and six extra false-detections. All of the additional false-detections result form the periodic discharges of subject 36. The close performance of both detectors in terms of latency suggests that the maximum-likelihood classifier in the SDP architecture ignored, to a great extent, features from irrelevant derivations, and effectively exploited those crucial for detection of seizure onset. The results suggest that the exemplary SDP architecture under study did not exploit inter-derivation relations masked or lost by the independent processing of the SIP architecture.

The ability of a maximum-likelihood classifier to ignore features irrelevant for determining the class membership of an observed feature vector can be shown by re-expressing the likelihood ratio that the classifier compares to a threshold to classify a feature vector. To observe this, consider classifying a two-dimensional feature vector X=[x₁ x₂] as an instance of the classes C₁ or C₂ when the feature x₁ is identically distributed conditioned on both classes, and is also independent of x₂. A decision rule of this case can be represented as follows:

$$if \frac{p ( X \vert {C}_{1} )}{p ( X \vert {C}_{2} )} \geq γ then X ∈ {C}_{1}$$

Since the likelihood of X in this case can be re-expressed as p(X)=p(x₁|x₂)p(x₂)=p(x₁)p(x₂), the decision rule can be rewritten as:

$$if \frac{p ( {x}_{1} \vert {x}_{2} , {C}_{1} ) p ( {x}_{2} \vert {C}_{1} )}{p ( {x}_{1} \vert {x}_{2} , {C}_{2} ) p ( {x}_{2} \vert {C}_{2} )} = \frac{p ( {x}_{1} \vert {C}_{1} ) p ( {x}_{2} \vert {C}_{1} )}{p ( {x}_{1} \vert {C}_{2} ) p ( {x}_{2} \vert {C}_{2} )} \geq γ$$

$$then X ∈ {C}_{1}$$

Because x₁ is identically distributed conditioned on both classes, the likelihood p(x₁|C₁)=p(x₁|C₂) and the decision rule simplifies to one that relies only on the feature x₂ for classification:

$$if \frac{p ( {x}_{2} \vert {C}_{1} )}{p ( {x}_{2} \vert {C}_{2} )} \geq γ then X ∈ C .$$

More generally x₁ and x₂ need not be independent. In such as case, x₁ needs to be identically distributed conditioned on both classes and the feature x₁ for the above result to hold since p(X)=p(x₁|x₂)p(x₂)≠p(x₁)p(x₂). In other words, for the decision rule to reduce to one that only relies on x2, the stronger condition p(x₁|x₂,C₁)=p(x₁|x₂,C₂) needs to be satisfied.

FIG. 51B compares the performance of the exemplary SIP and SDP architectures when each is combined with support vector machine classifiers. The SDP architecture exhibits a smaller detection latency and a higher number of true-detections relative to the SIP architecture, but a greater number of false-detections. The smaller average detection latency of the SDP architecture suggests that the support vector machine to some extent was handicapped by the smaller feature vectors in the SIP architecture, and is more effective when allowed to freely exploit the interrelations of elements within larger feature vectors.

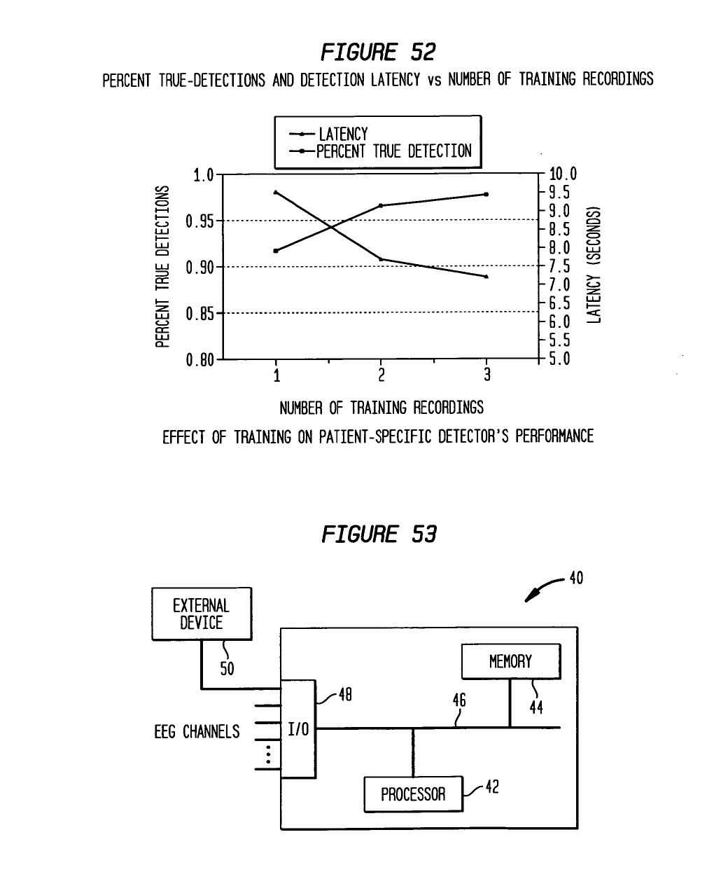

The performance of patient-specific seizure detector according to the teachings of the invention can be improved by providing the detector with additional training. By way of example and only for illustrative purposes, FIG. 52 illustrates the improvement in an exemplary patient-specific detector's average detection latency and true-detection rate as a function of the number of 20 minute EEC training recordings observed. Each training recording includes a single seizure event as well as non-seizure activity from a given subject. The figure highlights that an exemplary detector trained on one recording from a test subject is capable, on average, of detecting 91% of that subject's future seizures with a mean latency of 9.5+5.0 seconds. When an additional training recording was employed, the detector identified 96% of the subject's future seizures with a latency of 7.6±2.4 seconds. Utilizing a third recording only slightly improved the detector's performance beyond that was obtained by using two training recordings. In particular, a detector trained on three recordings detected on average 97% of a subject's future seizures with a mean latency of 7.1±1.9 seconds. A decrease in mean latency as well as a decrease in deviation about the mean was observed as the number of training records was increased. This data was compiled by employing twenty one of the thirty six test subjects employed in the above study, which explains the deviation of the true detection rates and average detection latencies from those presented above. False-detections were not greatly affected by the number of training records observed. Rather, they were primarily affected by the prevalence of a patient's seizure-like, interictal abnormalities and diversity of artifacts collected for inclusion in the training set.

This data indicates that a patient-specific detector of the invention can reliably and quickly detect seizure onsets even when with a few as two training seizures. This can be particularly advantageous in clinical settings in which data collection time can be short and the occurrence of seizure events in some patients can be rare.

A seizure detector according to the teachings of the invention can be implemented by utilizing a variety of hardware and software systems. For example, FIG. 53 schematically illustrates a detector 40 according to one embodiment of the invention in the form of a programmable computing device having a processing unit 42, and associated memory 44 in communications with the processor via a bus 46 in a manner known in the art. The exemplary computing device further includes an input/output (I/O) communications interface 48 having a plurality of ports for receiving EEG waveform data from a plurality of EEG channels. The I/O interface can also allow the computing device to communication with an external device 47, which can be, e.g., a display or a device utilized to program the computing device. The computing device can further include other components (e.g., amplifiers, etc) well known in the art (not shown) that provide functionalities needed for its operation.

A plurality of instructions identifying a seizure onset in accordance with the teachings of the invention, discussed in detail above, can be stored in the memory 44. These instructions can include, for example, information needed for extracting feature vectors from incoming data, classifying them and identifying a seizure onset based on the classification. In addition, the memory can store instructions for generating one or more decision parameters during the training stage. For example, the detector 40 can be trained by providing, it with a patient's training EEG recordings so that it can generate and store in the memory 44 one or more decision parameters to be utilized subsequently in identifying seizure onsets, in a manner described in detail above.

In some exemplary implementations, the computing device 40 can be a digital signal processor (DSP). In one embodiment, the DSP is programmed to execute instructions implementing various stages of a seizure onset detection method according to the teachings of the invention, including the feature extraction stage, and the classification stage, for example, by employing a support-vector machine previously trained on patient-specific examples of seizure and non-seizure EEG waveforms. As discussed above, the classification stage can incorporate spatial correlations among EEG waveform channels into the classification decision by examining features from all channels concurrently. The DSP can also be programmed to impose a selected temporal constraints for declaring a seizure onset. For example, in one embodiment, the temporal constraint requires that two sequential EEG epochs to be classified as members of the seizure class prior to declaring a seizure event. Requiring the persistence of seizure activity for two epochs helps avoid false detections due to short-time, seizure-like activity commonly observed between actual seizures.

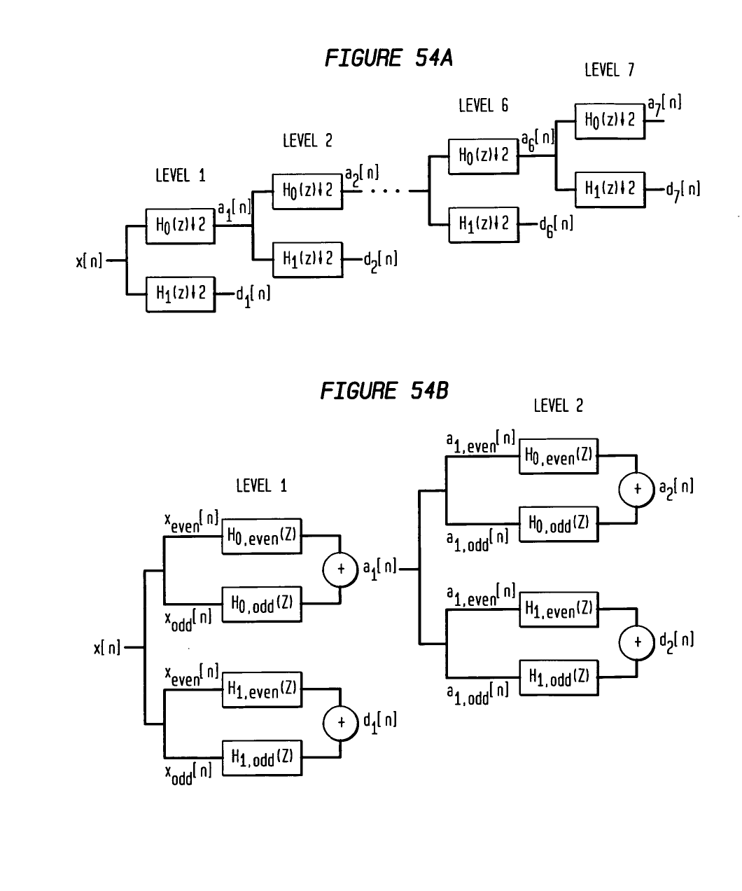

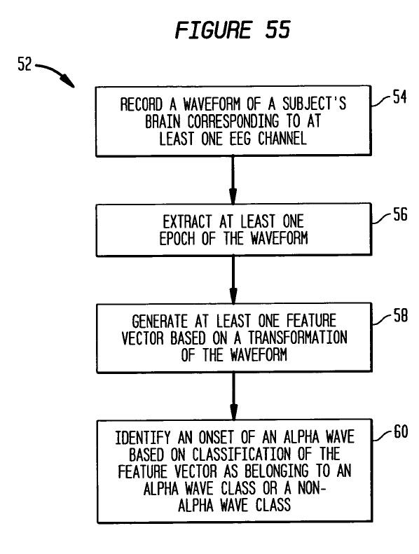

In some embodiments, the DSP extracts four features from each input channel, which correspond to that channel's energy in the 4^{th}-7^{th} levels of a multiscale wavelet decomposition, summarizing morphology of an epoch of that channel's waveform. In this embodiment, an epoch of the waveform is selected to have a 2.56 second time duration, and is digitized into 512 data points. The four features are not computed following the arrival of 512 data points from a particular channel. Rather, they are incrementally computed 2 data points at a time by utilizing the iterated filter bank shown in FIG. 54A. In this embodiment, the filterbank sequentially filters and downsamples an input sequence x[n] by utilizing 8-point impulse responses h₀[n] and h₁[n] in order to produce the wavelet decomposition d[n] at level i, and the approximation coefficients a[n] at level i. The absolute sum of the wavelet coefficients at level i corresponds to the input signal's energy at that level. Furthermore, the downsampling that occurs between levels implies that the two approximation coefficients aᵢ[n] and aᵢ[n+1] are necessary to compute the wavelet coefficients d₁₊₁[n] and the approximation coefficient aᵢ₊₁[n].

When the first two data point {x[1],x[2]} arrive they are filtered and downsampled to generate the first-level coefficients {d₁[1],a₁[1]}. The arrival of the next two data points {x[3],x[4]} permits the computation of the next set of first-level coefficients {d₁[2],a₁[2]}. Now the pair of first level approximation coefficients {a₁[1],a₁[2]} can be filtered and downsampled to generate the second-level coefficients {d₂[1],a₂[1]}. The third-level coefficients can be generated in the same manner. The pair-wise processing of {x[5],x[6]} and {x[7],x[8]} produces {d₁[3],a₁[3]} and {d₁[4],a₁[4]}. The subsequent filtering of {a₁[3],a₁[4]} produces the coefficients {d₂[2],a₂[2]}. Finally, propagating the coefficients {a₂[1],a₂[2]} leads to the first set of third-level coefficients {d₃[1],a₃[1]}.

The ongoing arrival and propagation of pairs of input samples permits the computation of increasingly higher level wavelet coefficients. In particular, the arrival and propagation of the two samples {x[127],x[128]} through the filterbank leads to computing the first wavelet coefficient at the seventh level d₇[0]. By the time the 512 input data points have arrived, 512/2ⁱ coefficients will have been computed at the levels of interest i=4, 5, 6, 7. The absolute sum of the coefficients at each of these levels completes the incremental computation of the four features for a single input channel. The DSP carries out these computations for each of the input channels so as to construct a composite feature vector.

The computational methodology outlined above is more efficient than computing the wavelet coefficients of the 4^{th}-7^{th} levels via transfer functions that directly map the input data point (512 in this embodiment) sequence x[n] to the coefficients dᵢ[n], i=4, 5, 6, 7—though the latter approach can be employed in other embodiments. In fact, the direct mapping of a 512 point input sequence to d₄[n] using the 106 point impulse response of the associated transfer function would require 10240 operations using radix-2 FFTs. In contrast, the method outlined above requires 5888 operations when using time domain convolutions. The outlined method requires fewer operations because it exploits inter-level downsampling and convolutions with the short, 8-point impulse responses h₀[n] and h₁[n].

Alternatively, the wavelet coefficients of the 4^{th}-7^{th} levels, whose absolute sum represents the energy at these levels, are computed M data points at a time by using a polyphase implementation of a seven-level wavelet filterbank and overlap-add convolution. Computing the features of each channel is completed after processing only 512/M buffers. FIG. 54B illustrates the first two levels of such a filterbank that can be used to compute the wavelet coefficients of a channel-independent filterbanks are used to compute the wavelet coefficients of each channel.

In this embodiment, the DSP determines the class membership of a feature vector by evaluating a support-vector machine classification in real-time. More specifically, a feature vector X is assigned to the seizure class if the condition in Equation (11) below holds, otherwise the feature vector is assigned to the non-seizure class.

$$( {\sum }_{j = 1}^{N} {α}_{j} exp \frac{{‖ X - {X}_{j} ‖}^{2}}{σ D} ) + β > T . \qquad Equation ( 11 )$$

Similar to the previous embodiments, the support-machine classification rule is parameterized by the coefficients αⱼ, the support-vectors Xⱼ, the radial-basis kernel parameter σ, the feature vector dimension D, the summation limit N, and the bias β, and T is a pre-selected threshold, which in some embodiments can be chosen to be zero. These parameters are computed offline while training the support-vector machine to differentiate between a subject's seizure and non-seizure EEG. Once the parameters are determined, they are downloaded onto the DSP to allow real-time classification of newly observed feature vector.

More generally, the teachings of the invention can be employed to detect a change in a subject's EEG waveform, observed through a time period, based on spatial and morphological features of the waveform. For example, in another aspect, the invention provides methods and systems for detecting onset of alpha waves in a subject. With reference to a flow chart 52, in one embodiment of a method of the invention for detecting onset of a subject's alpha waves, in step 54, a waveform of the subject's brain corresponding to at least one channel of EEG measurement is monitored. In step 56, at least one sample (one epoch) of the waveform is extracted and at least one feature vector based on a transformation (e.g., time-frequency transformation) of the sampled waveform is generated (step 58). In step 60, an onset of an alpha wave is identified by classifying the feature vector as belonging to a non-alpha wave class or an alpha wave class based on comparison of the feature vector with at least one reference value (decision measure) previously determined for that subject. The decision measure can correspond, for example, to a hyperplane generated based on support vectors computed from reference feature vectors obtained from reference alpha-wave and non-alpha wave EEG waveforms of the subject.

To show feasibility of utilizing the DSP seizure detector in an ambulatory setting, as well as the feasibility of detecting onset of alpha waves, and only for illustrative purposes, the following case studies are presented. It should be understood that these studies are provided only for illustrative purposes and not for necessarily indicating the optical performance of a seizure detector of the invention. In general, real-time operability of a seizure detector can require that the time needed to process M samples be less than the time taken for M new samples to arrive. The following time constraint was adopted for the studies:

T_{F}(M)+T_{C}(N)<T_{R}(M) Equation (12)

wherein T_{F}(M) represents the time spent propagating M samples through all the wavelet filterbanks, T_{C}(N) represents the time spent classifying a feature vector using a support-vector machine with N support-vectors, and T_{R}(M) represents the time taken to receive M new samples. In this embodiment, the quantity T_{F}(M)+T_{C}(N) represents the delay between the reception of the last M samples of 512 samples and classification of those 512 samples as belonging to the seizure or non-seizure class. The number of support-vector N is fixed by training the detector, but the buffer size M is freely chosen subject to inequality shown in Equation (12). To minimize the classification delay, the smallest M that would satisfy the real-time constraint was chosen, which can be a power of 2 less than or be equal to 512. More precisely, M can be determined by the following relation:

$$M = {min}_{K ∈ {2}^{n} n = 1 , … , 9} \{ K \vert {T}_{F} ( K ) + {T}_{C} ( N ) < {T}_{R} ( K ) \} \qquad Equation ( 13 )$$

In case (1) onset of alpha waves of a subject was detected by utilizing ambulatory EEG and case (2) seizure onset was detected in a stream of ambulatory EEG. In both cases, the temporal constraint typically imposed by the detector was disabled throughout the test process due to the short temporal profile of the studied EEG discharges. In case (1), ambulatory EEG was of a test subject captured by utilizing a DigiTrace™ 1800 Plus recorded (manufactured by SleepMed of Peabody, Mass., U.S.A.) and was streamed to the DSP at a rate of 200 sample data points/sec/channel. The DSP was tasked with detecting the onset of the first 10 Hz alpha waves within this live stream of data.

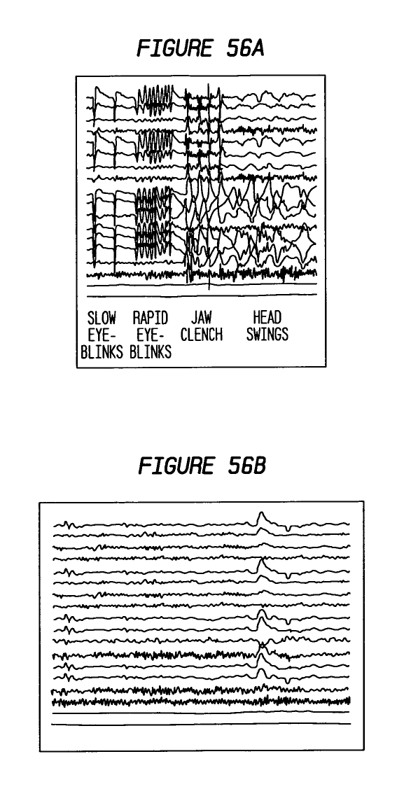

Prior to applying the DSP to the streaming EEG, it was trained on examples of alphawave EEG and non-alpha wave EEG waveforms recorded from the subject. The non-alpha wave EEG waveforms included baseline EEG as well as EEG corrupted by variable frequency eye blinking, jaw clinching, head swinging, electrode tapping and electrode head shaking. Training yielded N=18 support-vectors, which prescribes a buffer size of M=16. FIGS. 56A and 56B present, respectively, examples of non-alpha waves and alpha waves training EEG waveforms.

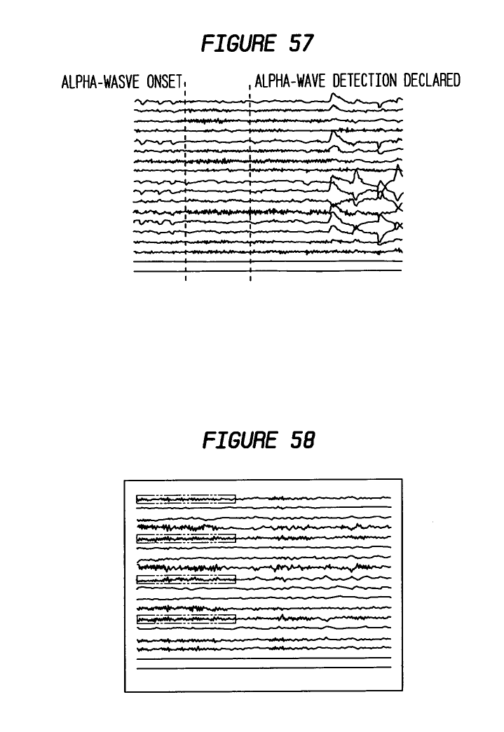

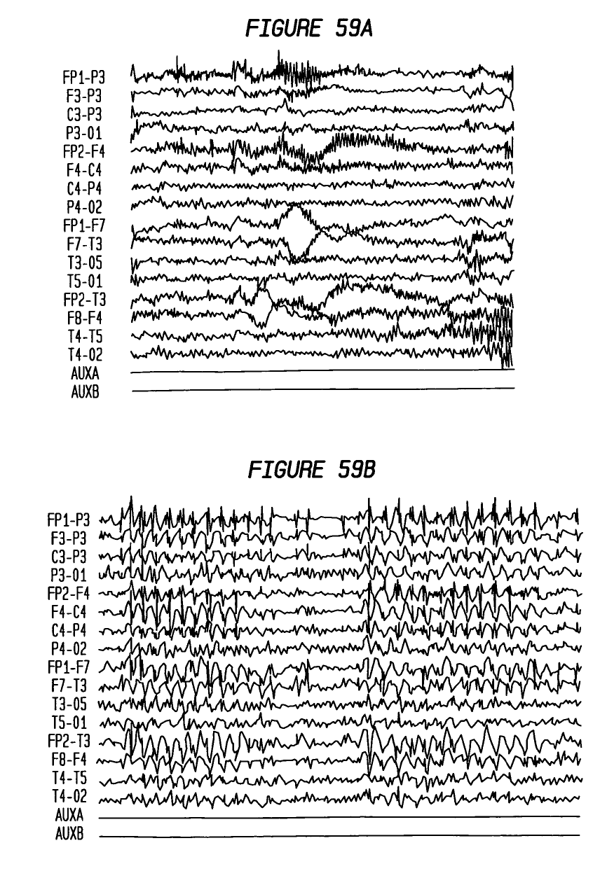

The trained detector was then used to process the subject's EEG in real-time as it was streamed by the ambulatory monitor. Movement and muscle artifacts did not result in any false-detections, and the onset of alpha-waves was detected within 2.56 seconds as shown in FIG. 57, by utilizing {C3-P3; C4-P4; T5-O1; T6-O2} channels. The observed latency in detecting alpha-waves onset is understood to be a byproduct of classifying EEG waveforms only after processing 2.56 second samples of information (512 data points). As a test of the spatial specificity of the detector, the inputs to the ambulatory recorder were switched so that alpha waves appeared on channels {FP1-F3, FP2-F4} rather than channels {C3-P3,C4-P4}, as shown in FIG. 58. In this configuration, the alpha-waves appropriately did not trigger any real-time detections.

In case (2), ambulatory EEG previously collected from a subject was streamed to the DSP at a rate of 200 sample data points/sec/channel. The EEG contained generalized 3-3.5 Hz spike-wave discharges lasting up to 5 seconds. These epileptiform events were not associated with any clinical correlates and can hence be considered short electrographic seizures. Prior to using the DSP to process the streaming EEG, the detector was trained on the epileptiform, baseline and artifact contaminated EEG waveforms. FIG. 59A shows examples of base line and artifact-contaminated training (reference) EEG waveforms while FIG. 59B shows an example of an training electrographic seizure that the detector is trained to recognize. Training yielded N=29 support vectors for use in classification, which prescribes a buffer size of M=16.

Two streams of data were sent to the DSP. The first stream consisted of 102 epochs each centered around an electrographic seizure and totaling 2.5 hours. The second stream consisted of 105 seizure-free epochs totaling 35 minutes (these were derived from 20-second EEG epochs captured every hours between 7 am-11 pm, and 20-second EEG epochs captured every 10 minutes between 11 pm-7 am over a 32 hour period). The seizure and non-seizure epochs were automatically created at the time of recording by the seizure event detector used in the DigiTrace™ 1800 Plus ambulatory unit.

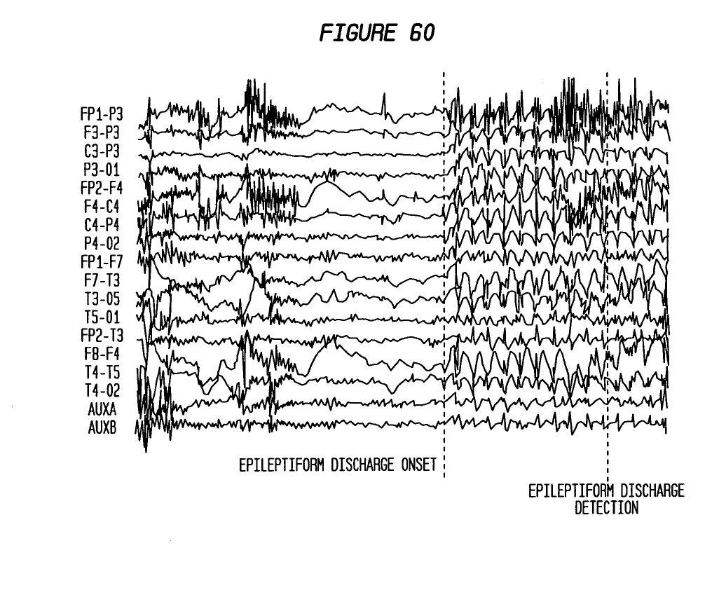

The trained DSP seizure detector detected all electrographic seizures of length 2.5 or more seconds in the 102 seizure epochs, but failed to detect discharges lasting between 1-2.5 seconds that were present in these epochs. No false detections were declared while processing the seizure-free epochs (the DSP, however, missed electrographic events lasting 1-2.5 seconds in this portion of the stream). By way of example, FIG. 60 shows detection of the onset of an epileptiform event within 3 seconds with no false detections on the preceding artifacts.

A composite feature vector that combines spatial and morphological features of a patient's EEG waveforms advantageously permits differentiating EEG signals of that patient corresponding to different spatial locations even if they manifest similar spectral properties. In other words, regional specificity exhibited by the EEG waveforms of a given subject (e.g., a observed waveform can be normal for one brain region of the subject but abnormal of another brain region of the same subject) can be employed in detection of a selected condition (e.g., seizure onset or abnormal alpha-wave detection) For example, a 10-Hz EEG signal that is centered over the occipital channels (with slight extension forward into parietal/central channels) of a subject can correspond to normal alpha waves while a similar 10-Hz signal that is predominantly localized to the temporal channels of the same subject can correspond to abnormal waveforms. Similar advantages can be obtained in seizure onset detection.

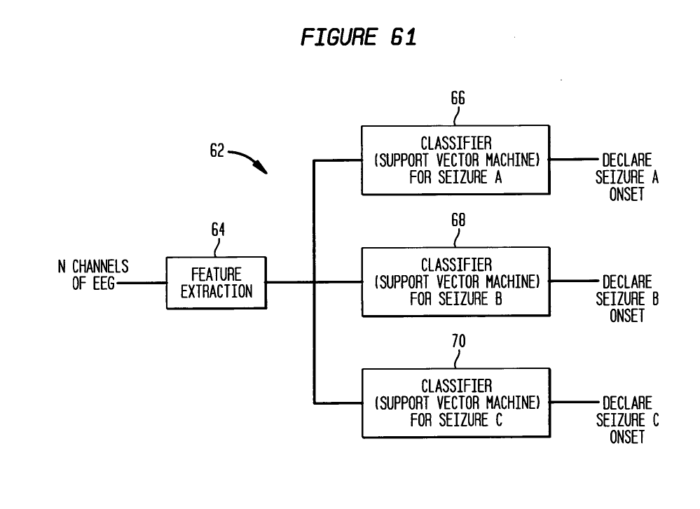

In some embodiments of the invention, a patient-specific seizure detector can not only identify onset of seizures in a patient but it can also assign the seizure to one of a plurality of seizure types (herein also referred to as seizure sub-classes). By way of example, FIG. 61 schematically illustrates an example of such a seizure detector 62 having a feature extractor 64 that receives one or more EEG waveform channels of a patient and generates feature vectors by applying a selected transformation (e.g., time-frequency transformation) to samples (epochs) of the waveforms. In this example, the detector includes three classifiers 66, 68, and 70, each of which is trained on a particular seizure type of the patient. For example, the classifier 66 is trained on seizure type A by providing it with reference EEG non-seizure waveforms as well as EEG waveforms of the patient corresponding to seizure type A. The classifier 66 can compute a decision measure based on the reference waveforms in a manner described above. The trained classifier 66 can then identify the onset of a seizure of type A by classifying the observed feature vector as belonging to a non-seizure class or a seizure class of type A. A similar training can be employed for classifiers 68 and 70—albeit by utilizing reference seizure waveforms of types B and C, respectively. The trained classifier 68 can then identify an onset of a seizure of type B based on classification of an observed feature vector as belonging to a non-seizure class or a seizure class of type B, and the trained classifier 70 can identify an onset of a seizure of type C based on classification of an observed feature vector as belonging to a non-seizure class or a seizure class of type C. It should be understood that additional classifiers corresponding to other seizure types can also be added to the detector architecture shown in FIG. 61.

The seizure-onset detection methods and systems described above can find a variety of diagnostic and therapeutic applications. Some examples of such applications include, without limitation, the use of a variety of patient imaging modalities, delivery of diagnostic and/or therapeutic agents and stimuli in combination with seizure onset detection according to the teachings of the invention, as discussed in more detail below.

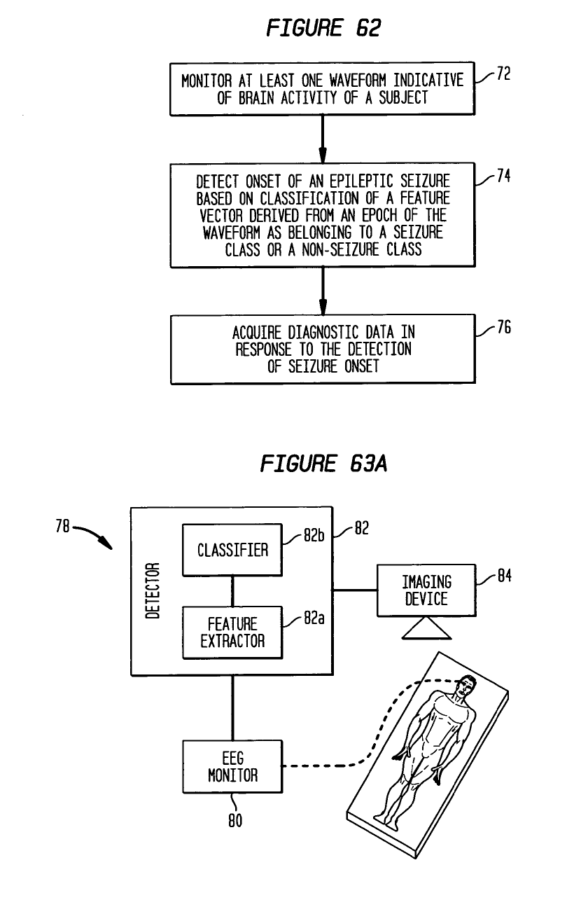

For example, with reference to a flow chart of FIG. 62, in one aspect, the invention provides a method for acquiring diagnostic data from a patient by monitoring in step 72 at least one waveform indicative of brain activity of the patient. In step 74, an onset of an epileptic seizure of the patient is detected by classifying at least one feature vector corresponding to a sample of the waveform as belonging to a seizure or a non-seizure class. This classification can be based on comparison of the feature vector with a measure derived from previously-observed seizure-related and non-seizure waveforms of that patient in a manner described above. In step 76, diagnostic data can be acquired in response to the seizure onset detection.

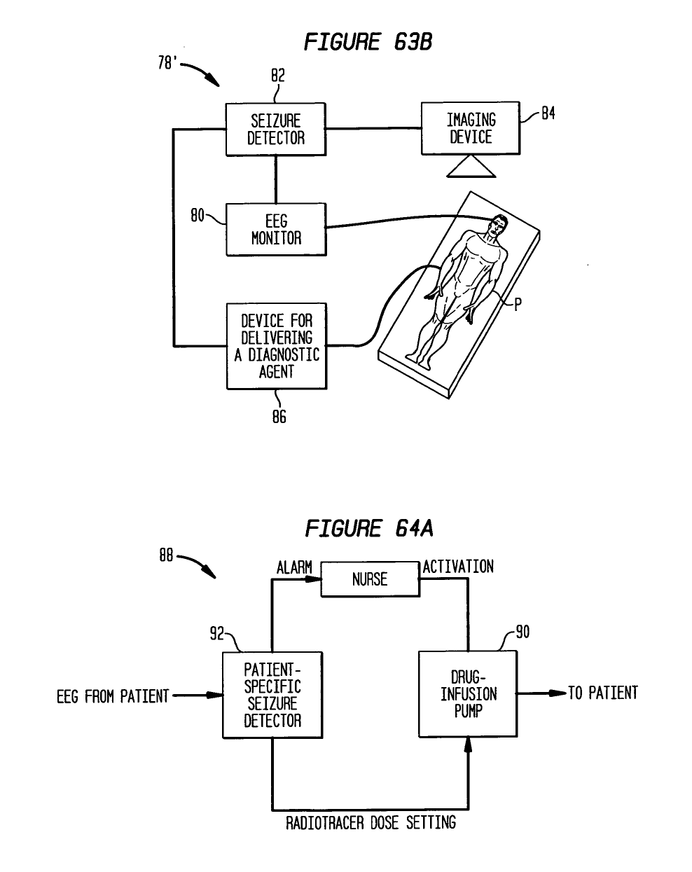

In some applications, the above diagnostic data acquisition can be implemented to form an imaging device that can provide an image of a subject (a patient) in response to a detected seizure onset. By way of example, FIG. 63A schematically depicts an imaging system 78 according to an embodiment of the invention that includes an EEG monitor device 80 for acquiring EEG brain waveforms of a subject. In many embodiments, the EEG monitor device can be of the type conventionally employed for obtaining non-invasive EEG measurements. The exemplary system 78 further includes a seizure detector 82 according to the teachings of the invention that can be coupled to the EEG monitor to receive one or more waveform channels therefrom. The seizure detector employs the waveforms in a manner described above to identify an onset of a seizure.

More particularly, with continued reference to FIG. 63A, the seizure detector 82 can have a feature extractor 82a that applies a selected transformation (e.g., a wavelet transformation) to the received waveforms to generate one or more feature vectors in a manner discussed above. In addition, the detector can include a classifier 82b, previously trained on seizure and non-seizure EEG waveforms of the patient, that can identify a seizure onset based on the classification of the feature vectors.

The imaging system can further include an imaging device 84 that can acquire an image of at least a part of the patient in response to the detection of a seizure onset by the detector. In some embodiments, the detector can issue, upon detecting a seizure onset, a notification (e.g., an alarm) to a human operator who can activate the imaging device, in response to the notification, to start acquiring images of the patient. In other embodiments, the detector can include an activation circuitry, coupled to the imaging device, that automatically triggers the imaging device to begin collecting images of the patient in response to detection of a seizure onset. The detector can trigger the imaging device as soon as a seizure onset is detected. Alternatively, the detector can delay triggering the imaging device for a selected time period after detection of a seizure onset.

With reference to FIG. 63B, in some embodiments, an imaging system 78′ according to the teachings of the invention can further include a device 86 for delivering a diagnostic agent to a patient (P) so as to facilitate acquiring the patient's images. The delivery system can apply the diagnostic agent to the patient upon detection of a seizure onset by the seizure detector 82. For example, the detector can issue a notification (e.g., an alarm) upon detecting a seizure onset to a human operator (not shown) who can in turn trigger the delivery device to apply the agent to the patient. More preferably, the delivery device 86 operates under the control of the detector. In such a case, the detector can effect triggering of the delivery device automatically in response to the detection of a seizure onset to deliver the agent to the patient. In some embodiments, the detector can provide the delivery device with information regarding a dose of the diagnostic agent to be delivered to the patient.

A variety of delivery devices and diagnostic agents known in the art can be employed in the system 78. For example, the delivery device can be an infusion pump that can infuse the diagnostic agent, e.g., a dye or a radiotracer, into the patient.

Moreover, a variety of imaging systems known in the art can be employed in the above exemplary systems 78 and 78′. For example, in some embodiments, the imaging system can provide an image of a metabolic activity in a selected anatomical portion (e.g., brain) of the patient. Alternatively or in addition, the imaging system can provide an image of neural activity in at least a portion of the patient's brain. Some examples of suitable imaging devices can include, without limitation, devices for performing single-photo-emission computed tomography (SPECT), functional magnetic resonance imaging (fMRI) and near infrared spectral imaging (NFSI). In other embodiments, an imaging system for performing magnetoencephalography (MEG), a non-invasive diagnostic modality for functional brain mapping, can be employed.

In some embodiments, the imaging device 84 provide ictal SPECT image (scan) of the patient's brain. Ictal SPECT is a functional imaging procedure that can be used to localize or lateralize the focus of a seizure. It typically requires the injection of a radiotracer near the electrographic onset of a seizure prior to imaging for precise seizure focus localization. As the potential time window during which the radiotracer can be administered for an ictal SPECT can last a few hours (e.g., 6 hours), conventionally a nurse relies on notification from a caregiver or a patient regarding onset of clinical manifestations of a seizure. Upon receiving the notification, the nurse determines a dose of a radiotracer to be administered to the patient, and infuses that dose to the patient. The radiotracer dose depends on how much time has elapsed from the time when it was prepared. For example, the dose for imaging a seizure occurring within the first hour of the study can be different than the one for imaging a seizure occurring within the last hour of the study. This protocol, however, results in appreciable injection delays because the seizure's clinical onset typically lags behind its electrographic onset. Further, early signs of the seizure's clinical onset are subtle and the trained nurse is typically far from the patient. In many cases, injections are started 25 to 55 seconds after the onset of clinical signs. Such delays often lead to poor localization of the epileptogenic focus due to the visualization of secondarily activated foci in addition to the primary seizure focus.

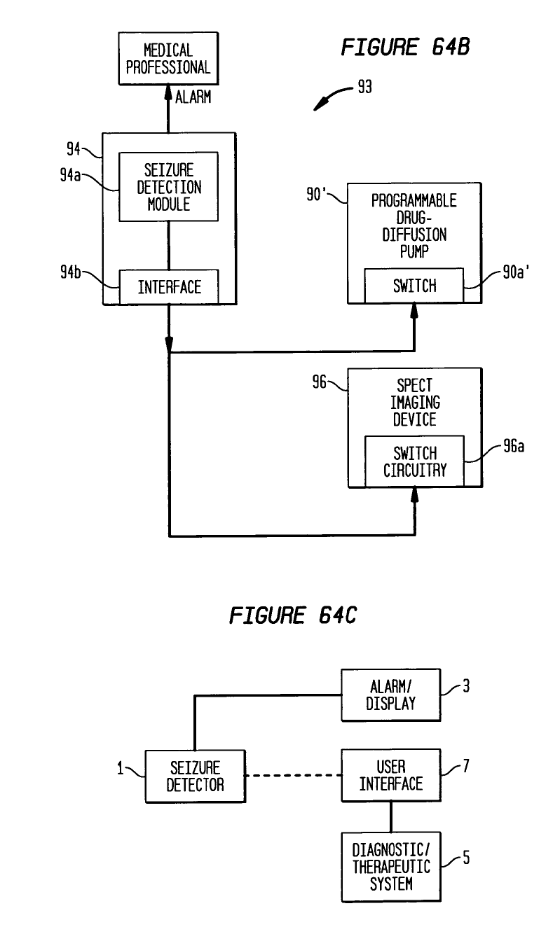

An ictal SPECT imaging system according to the teachings of the invention advantageously reduces delays between onset of a seizure and injection of a radiotracer and acquisition of an image by automatically detecting the seizure onset by employing the methods and systems of the invention for seizure onset detection, such as those described above. For example, FIG. 64A schematically depicts a system 88 according to one embodiment of the invention for administering a radiotracer to a patient, via an infusion pump 90, in response to detection of a seizure onset. The exemplary system 88 includes a patient-specific seizure detector 92 according to the teachings of the invention that can identify onset of a seizure in a patient under study via monitoring in real-time one or more EEG waveforms of the patient. The details of such detectors were previously provided above, and hence are not repeated. Upon detecting a seizure onset, the detector can alert a medical professional (e.g., nursing staff) by employing, for example, an audio and/or visual alarm. In addition, the detector can set the radiotracer dose to be injected into the patient. For example, the detector can include a module (not shown) for computing the dose based on well-known protocols, and a module (not shown) for communicating the calculated dose to the pump. Such modules can be constructed by employing techniques well known in the art. In response to the alert received from the detector, the medical professional can activate the infusion pump, e.g., remotely from a workstation, to administer the radiotracer dose to the patient. An ictal SPECT scan of the patient's brain can then be initiated. The medical professional can also decide not to activate the pump and await another notification from the detector.

With reference to FIG. 64B, in an alternative embodiment of an ictal SPECT imaging system 93 of the invention, a patient-specific seizure detector 94 can not only automatically program a programmable infusion pump 90′ to set a dose of the radiotracer in response to detection of a seizure onset, but it can also automatically cause activation of the pump to administer the radiotracer to the patient. The exemplary seizure detector 94 can include a detection module 94a for identifying a seizure onset and an interface module 94b that can communicate with the pump via a communications interface thereof to set the dose of the radiotracer and activate the pump, e.g., via a switching module 90′a of the pump. In addition, in this exemplary embodiment, the detector's interface module can communicate with the SPECT imaging device 96 to automatically initiate the imaging process after injection of the radiotracer, e.g., with a selected delay relative to the injection of the radiotracer. For example, the detector can transmit a trigger signal to a switching circuitry 96a of the imaging device to initiate a SPECT scan. Moreover, similar to the previous embodiment, the detector can notify a medical professional that a seizure onset has been detected. The various communications and switching modules shown in FIG. 64B can be constructed by employing well-known techniques without undue experimentation.

With reference to FIG. 64C, in some embodiments, upon detection of a seizure onset in a patient by a seizure detector 1 constructed according to the teachings of the invention, one or more waveform channels identified as exhibiting seizure activity as well as previously-obtained reference waveforms corresponding to those channels are presented via a display device 3 to a medical professional (an alarm can accompany the display) who can decide whether or not to activate a diagnostic and/or therapeutic system 5 (e.g., the pump of and/or the imaging device associated with an ictal SPECT system) based on comparison of the waveforms. For example, if the medical professional determines that the identified seizure corresponds to a false-positive (based on comparison of the corresponding waveform(s) with the reference waveform(s)), she will not activate the diagnostic/therapeutic system 5. Alternatively, the medical professional can utilize a user interface 7 to activate the diagnostic/therapeutic system 5. The reference waveforms can correspond, for example, to previously-observed seizure events of that subject. Alternatively, or in addition, the reference waveforms can correspond to inter-ictal discharges previously observed in that patient. By way of example, the medical professional may decide that a detected seizure event in fact corresponds to an inter-ictal discharge (based on comparison of detected waveforms with previously-obtained inter-ictal discharge waveforms of the patient), and hence is a false-positive detection. It should, however, be understood that in some cases, the detection of inter-ictal discharges may be desired.

In addition, the medical professional (or other qualified personnel) can employ the user interface 7 to reset and/or update the seizure detector 1. For example, the detector's training set can be updated to minimize, and preferably avoid, such false-positive detections in the future.

In some imaging applications, the methods and systems of the invention for automatically identifying seizure onsets are utilized to correlate seizure events of a patient with one or more images of that patient. For example, in one embodiment, one or more EEG waveform channels of a patient are recorded during a selected time period. During at least a portion of that time period, and preferably throughout the entire period, an image of a patient, e.g., a video image, is also recorded. The EEG waveforms can then be employed to automatically detect seizure events, if any, of that patient during that time period by applying the above-described methods. A detected seizure event, or at least a portion thereof, can then be correlated with at least one time segment of the recorded image. The detection of the seizure events and their correlation with the image can be performed by post-processing of the recorded EEG and image. Alternatively, they can be performed in real-time as the EEG and the image are recorded.

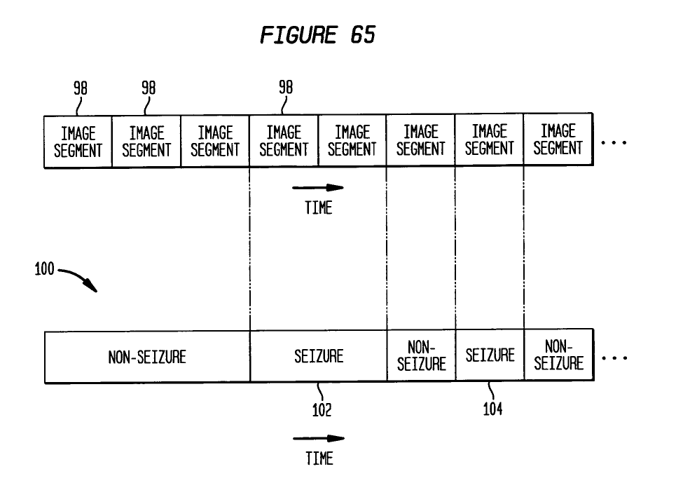

By was of example, FIG. 65 shows schematically a plurality of image segments 98 comprising a video image of a patient, and it further schematically represents an EEG recording of that patient obtained concurrently with the video image indicating two seizure events 102 and 104. In one embodiment, subsequent to recording the EEG and the video image, a seizure detector of the invention identifies the seizure events within the EEG recording, and hence permits identifying the time at which each seizure event occurred. This in turn allows correlating each seizure event with a particular segment of the video. To further facilitate the correlation of the seizure events with segments of the video image, the seizure detector can also identify the termination of each seizure and hence the duration of each seizure event. The identification of the termination of seizure can be accomplished by utilizing the above methods by recognizing a change from a seizure EEG morphology to normal EEG morphology.

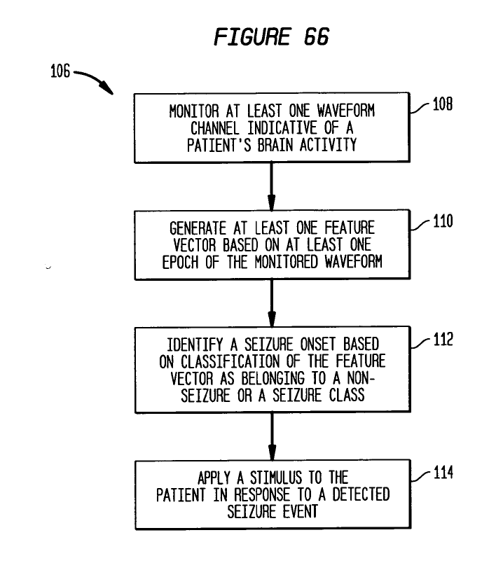

In other aspects, the present invention provides methods and systems for applying a stimulus to a subject in response to detection of onset of a seizure in that subject. For example, with reference to a flow chart 106 of FIG. 66, in such a method, in step 108, at least one waveform channel indicative of a subject's brain activity is monitored. The brain waveform can correspond to non-invasive or invasive EEG waveform of the subject. In step 110, at least one feature vector is generated based on at least a sample (epoch) of the monitored waveform. An onset of a seizure can then be identified by classifying the feature vector as belonging to a seizure class or a non-seizure class by comparison with a measure derived from previously-observed seizure and non-seizure brain waveforms of that subject (step 112). The construction and classification of the feature vector, as well as the use of the classification in identifying a seizure onset, were discussed in detail above. In step 114, a stimulus is applied to the patient in response the detected seizure onset. The stimulus can be, for example, an electromagnetic excitation or a pharmacological agent.

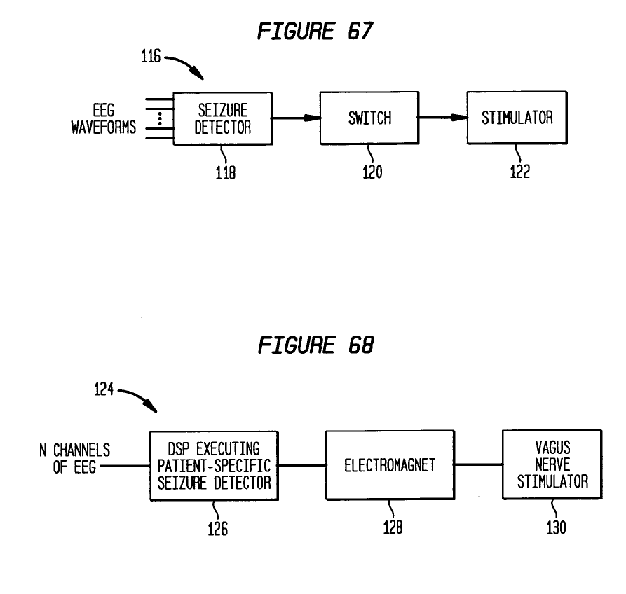

By way of example, the above method for applying a stimulus to a subject can be implemented by an exemplary system 116, schematically depicted in FIG. 67. The exemplary system 116 includes a seizure detector 118 in accordance with the teachings of the invention that is adapted to receive one or more EEG waveform channels of a patient. The detector can be trained, for example, in a manner discussed above, to detect onset of a seizure in that patient. The detector can further trigger a switch 120 coupled thereto in response to a detected seizure onset in order to activate a stimulator 122. Upon activation, the stimulator 122 can provide a stimulus to the patient. In some embodiments, the stimulus can include an electromagnetic excitation applied to the patient, while in others it can be a therapeutic agent, e.g., a pharmaceutical agent. Further, in some embodiments, the detector can delay triggering the switch for a selected time period after detecting a seizure onset. In some embodiments, rather than automatically activating the stimulator in response to a detected seizure, the detector 118 generates an alarm upon detecting a seizure onset to notify a human operator who can decide whether to activate the stimulator.

In some embodiments of the invention, the stimulator is a vagus nerve stimulator (VNS) that can provide a selected excitation to the patient's vagus nerve in response to detection of a seizure onset. Vagus nerve stimulators suitable for use in the system 116 are known in the art. Briefly, a VNS system can include a plurality of nerve electrodes that are implanted on selected portions of a patient's vagus nerve. The nerve electrodes preferably include tethers for maintaining them in place without undue stress on the coupling of the electrodes onto the nerve. The VNS also includes an implantable neurostimulator (a pulse generator) that can be implanted in the patient, e.g., in the chest or axillary regions, so as to be in electrical communication with the electrodes to apply excitation pulses thereto.

The pulse generator can be activated externally by employing a variety of techniques. For example, the generator can include a reed switch that can be activated by an external magnet. In some embodiments of the invention, the detector can automatically cause activation of the pulse generator via a switch (e.g., an electromagnet), as discussed in detail below. In other embodiments, the detector, rather than automatically activating the pulse generator, provides a notification (e.g., an alarm) to a medical personnel or the patient upon detection of a seizure onset. The medical personnel or the patient can then employ an activation mechanism, e.g., a magnet, to activate the pulse generator.

The pulse generator can be programmed to apply selected excitation pulse or pulses to the patient upon activation. Some exemplary excitation pulse parameters suitable for use in the practice of the invention can include, without limitation, pulse widths in a range of about 130 to about 1000 microseconds, pulse currents in a range of about 0.25 mA to about 3.5 mA, pulse repetition frequencies (signal frequency) in a range of about 1 Hz to about 30 Hz, pulse on-time in a range of about 7 seconds to about 60 seconds, and pulse off-time in a range of about 0.2 seconds to about 180 minutes (or infinite).

Further details regarding VNS systems and methods for their activation can be found, for example, in U.S. Pat. Nos. 5,154,172, 5,304,206 and 6,622,047, all of which are herein incorporated by reference in their entirety. A suitable VNS is marketed by Cyberonics, Inc. of Houston, Tex., U.S.A. under the trade designation VNS Therapy™ System. The Cyberonics system can provide automatic stimulation (normal mode) or on-demand stimulation (magnet mode). Typical stimulation parameters of this system are provided in Table 1 below:

TABLE 1

| Stimulation Parameters | Normal Mode | | Magnet Mode | |
|---|---|---|---|---|
| Output Current | 0-3.5 | mA | 0-3.5 | mA |
| Frequency | 30 | Hz | 30 | Hz |
| Pulse Width | 500 | μsec | 500 | μsec |
| ON Time | 30 | sec | 30 | sec |

| | | | |
|---|---|---|---|
| OFF Time | 5 | min | N/A |

As noted above, in some embodiments, a vagus nerve stimulation system of the invention provides a switch, e.g., an electromagnet, coupled between the automatic seizure-onset detector and a vagus nerve stimulator that automatically activates the VNS pulse generator in response to a signal received from the detector. Such an automated vagus nerve stimulation system that not only automatically detects the onset of a seizure but also activates a vagus nerve stimulator in response to a detected seizure without human intervention can be implemented in some embodiments of the invention as a portable system. For example, FIG. 68 schematically depicts such a portable vagus nerve stimulator system according to one embodiment of the invention that includes a digital signal process (DSP) 126 (e.g., a DSP manufactured by Texas Instruments of Dallas, Tex., U.S.A. under trade designation TM320C6711) having a plurality of input ports for receiving a number of EEG waveform channels. The DSP 126 can be programmed to implement the methods of the invention for detecting a seizure onset of a patient by operating on the inputted EEG waveforms of that patient. In addition, in this embodiment, the DSP 126 is connected to a battery-powered electromagnet 128, which when charged generates a magnetic field that is sufficiently strong to activate the pulse generator of a vagus nerve stimulator 120 at a distance (e.g., about 0.5 inches away from generator). More specifically, the DSP seizure detector charges the electromagnet upon detection of a seizure onset, thereby activating the VNS pulse generator. By way of example, in this embodiment, the electromagnet was employed to activate the above-referenced VNS pulse generator of Cyberonics in its on-demand mode at a distance of about 0.5 inches from the generator.

The stimulation of the patient's vagus nerve in response to detection of a seizure onset can prevent or lessen the severity and/or duration of the symptoms and signs of seizure. Further, such vagus nerve stimulation can ameliorate the severity and/or duration of the post-ictal (post-seizure) symptoms and signs. The stimulation of the patient's vagus nerve in response to detection of a seizure onset can potentially improve that patient's seizure frequency overall, i.e., enhance the prophylactic effect of vagus nerve stimulation.

More generally, a simulation can be applied to one or more of the subject's cranial nerves in response to detection of a seizure. For example, such excitation can be applied to the subject's glossopharyngeal nerve (ninth cranial nerve). It has been reported in animal models that the excitation of the glossopharyngeal nerve can shorten seizure durations (See, e.g., an article entitled “Ninth Cranial Nerve Stimulation for Epilepsy Control. Part 1: Efficacy In An Animal Model” authored by Patwardhan R. V., Tubbs R. S., Killingsworth C. R., Rollins D. L., Smith W. M, and Ideker R. E., and published in Pediatric Neurosurgery, 36(5), 236-243 (May 2002); which is herein incorporated by reference).

In other applications, electrical stimulation can be applied to the subject's brain tissue in response to detection of a seizure onset. By way of example, such stimulation can be applied by employing an intracranially implanted stimulator. U.S. Pat. No. 6,597,954, which is herein incorporated by reference, describes such a stimulator.

Some examples of stimulations that can be employed in the practice of the invention include, without limitation, simulation of the centromedian thalamic nuclei, part or parts of cerebellum, head of the caudate nucleus, cortical sites of seizure onset (such as neocortex, hippocampus, and temporal mesiobasal regions), anterior nucleus of the thalamus, or subthalamus. See, e.g., Chkenkeli et al. (2004), Clin Neurol Neurosurg 106: 318-329; Kerrigan et al. (2004), Epilepsia 45: 346-354; and Thoodore and Fisher (2004), Neurol 3: 33 (all of which are herein incorporated by reference). In some other embodiments, a stimulus (e.g., electrical excitation) can be applied to selected skin areas of a subject upon detection of a seizure onset in that subject. For example, stimulating the sections of the skin innervated by the vagus nerve can have therapeutic value.

In some embodiment, rather than applying an electrical stimulation to the subject, an anti-epileptic drug, such as but not limited to a benzodiazepine (for example, valium or lorazepam) or a barbiturate (such as Phenobarbital), is administered to the patient upon detection of a seizure onset.

In some embodiments, the vagus nerve stimulation can also be applied to a subject upon detection of inter-ictal discharges, which were discussed above.

The diagnostic and imaging methods and system described above in connection with seizure detection can also be utilized in combination with detection of onset of alpha waves. For example, a notification (an alarm) can be provided to a subject upon detection of onset of alpha waves in that subject.

All publications, including patents, reference herein are incorporated by reference in their entirety.

Those having ordinary skill in the art will appreciate the various changes can be made to the above embodiments without departing from the scope of the invention.

1-147. (canceled)

148. A method of determining a focus of a patient's epileptic seizure, comprising:
extracting at least a sample of at least one waveform monitoring neural activity in a selected brain portion of the patient,
identifying an onset of an epileptic seizure of the patient by classifying at least one feature vector as belonging to a seizure or a non-seizure class, said classification being based on comparison of the feature vector with a measure derived from previously-obtained reference brain waveforms of the patient, and
delivering a diagnostic agent to the patient upon detection of the onset of a seizure.

149. The method of claim 148, further comprising generating an image of said diagnostic agent.

150. The method of claim 148, further comprising selecting said diagnostic agent to be a radiotracer.

151. The method of claim 148, further comprising selecting said diagnostic agent to be dye.

152. The method of claim 150, further comprising generating a SPECT image of the patient's brain by utilizing said radiotracer.

153. The method of claim 152, further comprising employing said SPECT image to identify a focus of the seizure.

154. The method of claim 148, further comprising generating said feature vector based on a wavelet decomposition of said waveform sample.

155-160. (canceled)

161. A system for delivering a diagnostic agent to a patient, comprising:
a detector adapted to receive at least one waveform indicative of brain activity of a patient, said detector extracting at least a sample of said waveform and generating a feature vector corresponding to said sample,
said detector comprising a classifier trained on previously-obtained reference waveforms of the patient, said classifier identifying a seizure onset by classifying said feature vector as belonging to a seizure class or a non-seizure class based on comparison with a measure derived from said previously-obtained reference waveforms of the patient, and
a device for delivering a diagnostic agent to the patient in response to identification of a seizure onset.

162. The system of claim 161, further comprising a monitor device for generating said waveform data.

163. The system of claim 161, wherein said monitor device comprises any of a noninvasive or an invasive EEG measurement device.

164. The system of claim 161, wherein said detector causes activation of said delivery device upon identification of a seizure onset to deliver said agent to the patient.

165. The system of claim 161, wherein said delivery device comprises a pump for infusion of said diagnostic agent into the patient.

166. The system of claim 161, wherein said diagnostic agent comprises any of a radiotracer or a dye.

167. The system of claim 161, wherein said detector effects computation of a dose of the diagnostic agent to be delivered to the patient and communicates said computed dose to the delivery device.

168. A system for determining a focus of an epileptic seizure of a patient, comprising
a device for monitoring at least one EEG waveform channel of the patient,
a patient-specific seizure detector for detecting an onset of a seizure by classifying at least a feature vector derived from at least a sample of the waveform as belonging to a seizure or a non-seizure class, said detector performing the classification by comparing the feature vector with a measure computed based on one or more reference feature vectors previously derived for that patient, and
a pump for delivering a radiotracer to the patient in response to detection of a seizure onset by said detector.

169. The method of claim 168, wherein said detector effects activation of the pump upon detection of a seizure onset.

170. The system of claim 168, wherein said detector comprises
a feature extractor for decomposing said waveform sample into at least one subband signal and computing the feature vector as a function of energy contained within said subband signal, and
a classifier trained on reference EEG waveforms of said patient, said classifier assigning said feature vector to a seizure or a non-seizure class.

171. The system of claim 168, wherein said detector computes said feature vector as a composite of a plurality of feature vectors each corresponding to a sample of one of a plurality of EEG waveforms of the patient.

172-173. (canceled)
